SEQUENCE LISTING (32 sequences)

SEQ ID NO: 1 - DNA, 1792 bp, Homo sapiens
aattctgcagtcgacggtaccgcgggcccgggatccaccggtccgggatccaccggtccttgcgggccaccatgggcaactgggcggtgaacgagggcctgagcattttcgtgatcctcgtgtggctgggcctgaatgtctttctgttcgtgtggtactaccgcgtgtatgacatcccaccgaagtttttctatacccggaagctgctgggctccgctctcgccctggctagggccccggccgcctgcctgaatttcaattgcatgctcatcctgctgcccgtgtgtcggaacctgctgagcttcctgcgcgggtccagcgcctgctgcagcaccagggtgcgccgccagctggaccgcaacctcacgttccacaagatggtggcctggatgatcgccctgcatagcgccatccacacgatcgcccacctgttcaacgtggagtggtgcgtcaacgccagggtgaataacagcgacccatacagcgtcgccctctccgagctgggggaccgccagaacgagagctacctgaattttgcccgcaagcggattaagaaccctgaaggcgggctgtacctcgccgtgaccctgctggcgggcatcaccggcgtggtgattaccctgtgcctcatcctgatcatcaccagcagcaccaagaccatccgccgctcctacttcgaggtgttctggtacacccaccacctgttcgtgattttcttcatcggcctcgccattcacggcgccgagcgcatcgtgcggggccagaccgccgaatccctcgcggtgcacaacatcacggtgtgcgagcagaaaatctccgagtggggcaaaatcaaggaatgcccgatcccccagttcgcggggaatccccctatgacctggaagtggattgtgggccctatgttcctgtatctctgcgagcggctcgtcaggttttggcgcagccagcagaaggtggtcattaccaaagtcgtgacccacccatttaagaccatcgagctgcagatgaagaagaagggcttcaagatggaggtgggccagtacatcttcgtgaagtgtccgaaggtctccaagctggagtggcaccccttcaccctcacctccgctcctgaagaggatttcttctccatccacatccggatcgtgggcgactggaccgagggcctctttaacgcctgcgggtgcgacaagcaggagttccaggacgcctggaagctgcccaagatcgccgtggacggccccttcggcaccgccagcgaggacgtctttagctatgaggtggtcatgctggtgggcgccggcatcggggtgacccccttcgccagcatcctgaagagcgtgtggtataagtactgtaacaacgctaccaacctgaagctgaaaaagatttacttctactggctgtgtagggacacccacgcgttcgagtggtttgccgacctgctgcagctgctggagagccagatgcaggagcgcaacaatgccggcttcctgtcctacaacatctacctgaccgggtgggacgagagccaggcgaaccatttcgcggtgcaccacgacgaggagaaggatgtcatcaccggcctcaaacagaagaccctctacggcaggcccaactgggataacgagttcaagacgatcgctagccagcatcccaacacgaggatcggggtcttcctctgtgggcccgaggctctcgctgaaacgctctccaagcagtccatctccaactccgaatccgggccccgcggggtccatttcatcttcaacaaagaaaacttctgaggatccga

SEQ ID NO: 2 - DNA, 4035 bp, Homo sapiens
  CDS (63)..(1166)
gtgtagcgccagcgcgctgtgacgtaatgtgaggggtctcccggcagggctgagctggaccaatgaggaaaggcaaggggccgatttgcctgttctcacgccccaccctcagacctagccggagcaaagtttcacttatagaagggagaggagcgaacatggcagcgcgttggcggttttggtgtgtctctgtgaccatggtggtggcgctgctcatcgtttgcgacgttccctcagcctctgcccaaagaaagaaggagatggtgttatctgaaaaggttagtcagctgatggaatggactaacaaaagacctgtaataagaatgaatggagacaagttccgtcgccttgtgaaagccccaccgagaaattactccgttatcgtcatgttcactgctctccaactgcatagacagtgtgtcgtttgcaagcaagctgatgaagaattccagatcctggcaaactcctggcgatactccagtgcattcaccaacaggatattttttgccatggtggattttgatgaaggctctgatgtatttcagatgctaaacatgaattcagctccaactttcatcaactttcctgcaaaagggaaacccaaacggggtgatacatatgagttacaggtgcggggtttttcagctgagcagattgcccggtggatcgccgacagaactgatgtcaatattagagtgattagacccccaaattatgctggtccccttatgttgggattgcttttggctgttattggtggacttgtgtatcttcgaagaagtaatatggaatttctctttaataaaactggatgggcttttgcagctttgtgttttgtgcttgctatgacatctggtcaaatgtggaaccatataagaggaccaccatatgcccataagaatccccacacgggacatgtgaattatatccatggaagcagtcaagcccagtttgtagctgaaacacacattgttcttctgtttaatggtggagttaccttaggaatggtgcttttatgtgaagctgctacctctgacatggatattggaaagcgaaagataatgtgtgtggctggtattggacttgttgtattattcttcagttggatgctctctatttttagatctaaatatcatggctacccatacagctttctgatgagttaaaaaggtcccagagatatatagacactggagtactggaaattgaaaaacgaaaatcgtgtgtgtttgaaaagaagaatgcaacttgtatattttgtattacctctttttttcaagtgatttaaatagttaatcatttaaccaaagaagatgtgtagtgccttaacaagcaatcctctgtcaaaatctgaggtatttgaaaataattatcctcttaaccttctcttcccagtgaactttatggaacatttaatttagtacaattaagtatattataaaaattgtaaaactactactttgttttagttagaacaaagctcaaaactactttagttaacttggtcatctgattttatattgccttatccaaagatggggaaagtaagtcctgaccaggtgttcccacatatgcctgttacagataactacattaggaattcattcttagcttcttcatctttgtgtggatgtgtatactttacgcatctttccttttgagtagagaaattatgtgtgtcatgtggtcttctgaaaatggaacaccattcttcagagcacacgtctagccctcagcaagacagttgtttctcctcctccttgcatatttcctactgaaatacagtgctgtctatgattgtttttgttttgttgtttttttgagacggtctcgctgtgtcacacaggctggagtgcagtggcgtgagctcggctgactgcaaactctgcctcccaggtttaagcgattctcctgtcacagcttcccaagtagctgggatttacaggtgtgcaccgccatgccaggctaatttttgtgtttttagtagagacagggtttcgccaagttgtccaggctggtcttgaactcctgggctcaagtgatccgcccgcctcagtctcccaaagtgcgaggatgacatgtgtgagctaccacaccagcaatgtctatgcttctcgatagctgtgaacatgaaaagacatctattgggagtccgaggcaggtggattgcttgaggccaggagttagagaccagcctggccaacaaggcaaaaccccgtctctactaaaaatatgaaaattagctgggcttggtggctcatgcctataatcctagctacttgggaggctgaggcacgagacttgcttaatacctgggaggcggagattgcagtgagccgagatcacgctactgcgctccagcctgagtgatagagtgagactctgtctcaaaaaaaagtatctctaaatacaggattataatttctgcttgagtatggtgttaactaccttgtatttagaaagatttcagattcattccatctccttagttttcttttaaggtgacccatctgtgataaaaatatagcttagtgctaaaatcagtgtaacttatacatggcctaaaatgtttctacaaattagagtttgtcacttattccatttgtacctaagagaaaaataggctcagttagaaaaggactccctggccaggcgcagtgacttacgcctgtaatctcagcactttgggaggccaaggcaggcagatcacgaggtcaggagttcgagaccatcctggccaacatggtgaaaccccgtctctactaaaaatataaaaattagctgggtgtggtggcaggagcctgtaatcccagctacacaggaggctgaggcacgagaatcacttgaactcaggagatggaggtttcagtgagccaagatcacaccactgcactccagcctggcaacagagcgagactccatctcaaaaaaaaaaaaaaaagtaagaaagaaaaggactcccttagaatgggaaagaaaaatcataaaatattgagctgatgcctgtatatagaaattaagcgtttctcgaaagctgttctatgttttgctgttattttagtctttattctcttcctttaggtggagaaacaaagtaccaatttgaagggattttttttattttgtcttttggtttctgtcagtagaaataaccatatgtgctaaccaaatttctgtgaagaatgttttcatggttatcattatatctaactataacctcccccatagttatgaagagtaacctgaaatgccactattgtggaaataggataattgtaattgtgaaaaaataattttaaggaaatcttacaagtattacattaaaaagatactatgactgccacctgccatttaccttctaataaccctgccatgtggtttgcagaaagagatggatatagtagcctcagaagaaatattttatgtgggttttttgtttttcgttactagatttcatggatgaggggatatggttgaccttttactttttaatggagcagccagtttttgttaattactcacttgtaaattgtgagattctgaattccttacctgctattcttgtacttgtctcaggccaaatctatgctgtggttcttatgagacttgtatgaagatgccctgatttgtacagattgaccacgggaatactactgccatgtaatctgtatagttccagataatttgtcatgaacattgacagaatgacaattttttgtatttgctttttctccctttaagagcacattcttctgtaaggagaaaggcagcattctggctaaaatgtgtagaaggtaatttactacacttataaaatagtgtgacttttgtgaaaattttgaattagctttcatatgaagtgccttaagtagactcttcatttacttttctggtaatggtttaaatatcatttgttatgcatttttaagatacagttcagaatgacacattgtagtggcaaagataaccaaatgtctggctgtttgctttttgaccatatcaataaacttttacaatctaaaaaaaaaaaaaaaa

SEQ ID NO: 3 - protein, 367 aa, Homo sapiens
MRKGKGPICLFSRPTLRPSRSKVSLIEGRGANMAARWRFWCVSVTMVVALLIVCDVPSASAQRKKEMVLSEKVSQLMEWTNKRPVIRMNGDKFRRLVKAPPRNYSVIVMFTALQLHRQCVVCKQADEEFQILANSWRYSSAFTNRIFFAMVDFDEGSDVFQMLNMNSAPTFINFPAKGKPKRGDTYELQVRGFSAEQIARWIADRTDVNIRVIRPPNYAGPLMLGLLLAVIGGLVYLRRSNMEFLFNKTGWAFAALCFVLAMTSGQMWNHIRGPPYAHKNPHTGHVNYIHGSSQAQFVAETHIVLLFNGGVTLGMVLLCEAATSDMDIGKRKIMCVAGIGLVVLFFSWMLSIFRSKYHGYPYSFLMS

SEQ ID NO: 4 - DNA, 4353 bp, Homo sapiens
  CDS (62)..(1774)
attggaagaagaagcatagtatagaagaaaggcaaacacaacacattcaacctctgccaccatggggaactgggctgtgaatgaggggctctccatttttgtcattctggtttggctggggttgaacgtcttcctctttgtctggtattaccgggtttatgatattccacctaagttcttttacacaagaaaacttcttgggtcagcactggcactggccagggcccctgcagcctgcctgaatttcaactgcatgctgattctcttgccagtctgtcgaaatctgctgtccttcctcaggggttccagtgcgtgctgctcaacaagagttcgaagacaactggacaggaatctcacctttcataaaatggtggcatggatgattgcacttcactctgcgattcacaccattgcacatctatttaatgtggaatggtgtgtgaatgcccgagtcaataattctgatccttattcagtagcactctctgaacttggagacaggcaaaatgaaagttatctcaattttgctcgaaagagaataaagaaccctgaaggaggcctgtacctggctgtgaccctgttggcaggcatcactggagttgtcatcacgctgtgcctcatattaattatcacttcctccaccaaaaccatccggaggtcttactttgaagtcttttggtacacacatcatctctttgtgatcttcttcattggccttgccatccatggagctgaacgaattgtacgtgggcagaccgcagagagtttggctgtgcataatataacagtttgtgaacaaaaaatctcagaatggggaaaaataaaggaatgcccaatccctcagtttgctggaaaccctcctatgacttggaaatggatagtgggtcccatgtttctgtatctctgtgagaggttggtgcggttttggcgatctcaacagaaggtggtcatcaccaaggtggtcactcaccctttcaaaaccatcgagctacagatgaagaagaaggggttcaaaatggaagtgggacaatacatttttgtcaagtgcccaaaggtgtccaagctggagtggcacccttttacactgacatccgcccctgaggaagacttctttagtatccatatccgcatcgttggggactggacagaggggctgttcaatgcttgtggctgtgataagcaggagtttcaagatgcgtggaaactacctaagatagcggttgatgggccctttggcactgccagtgaagatgtgttcagctatgaggtggtgatgttagtgggagcagggattggggtcacacccttcgcatccattctcaagtcagtctggtacaaatattgcaataacgccaccaatctgaagctcaaaaagatctacttctactggctgtgccgggacacacatgcctttgagtggtttgcagatctgctgcaactgctggagagccagatgcaggaaaggaacaatgccggcttcctcagctacaacatctacctcactggctgggatgagtctcaggccaatcactttgctgtgcaccatgatgaggagaaagatgtgatcacaggcctgaaacaaaagactttgtatggacggcccaactgggataatgaattcaagacaattgcaagtcaacaccctaataccagaataggagttttcctctgtggacctgaagccttggctgaaaccctgagtaaacaaagcatctccaactctgagtctggccctcggggagtgcatttcattttcaacaaggaaaacttctaacttgtctcttccatgaggaaataaatgtgggttgtgctgccaaatgctcaaataatgctaattgataatataaataccccctgcttaaaaatggacaaaaagaaactataatgtaatggttttcccttaaaggaatgtcaaagattgtttgatagtgataagttacatttatgtggagctctatggttttgagagcacttttacaaacattatttcatttttttcctctcagtaatgtcagtggaagttagggaaaagattcttggactcaattttagaatcaaaagggaaaggatcaaaaggttcagtaacttccctaagattatgaaactgtgaccagatctagcccatcttactccaggtttgatactctttccacaatactgagctgcctcagaatcctcaaaatcagtttttatattccccaaaagaagaaggaaaccaaggagtagctatatatttctactttgtgtcatttttgccatcattattatcatactgaaggaaattttccagatcattaggacataatacatgttgagagtgtctcaacacttattagtgacagtattgacatctgagcatactccagtttactaatacagcagggtaactgggccagatgttctttctacagaagaatattggattgattggagttaatgtaatactcatcatttaccactgtgcttggcagagagcggatactcaagtaagttttgttaaatgaatgaatgaatttagaaccacacaatgccaagatagaattaatttaaagccttaaacaaaatttatctaaagaaataacttctattactgtcatagaccaaaggaatctgattctccctagggtcaagaacaggctaaggatactaaccaataggattgcctgaagggttctgcacattcttatttgaagcatgaaaaaagagggttggaggtggagaattaacctcctgccatgactctggctcatctagtcctgctccttgtgctataaaataaatgcagactaatttcctgcccaaagtggtcttctccagctagcccttatgaatattgaacttaggaattgtgacaaatatgtatctgatatggtcatttgttttaaataacacccaccccttattttccgtaaatacacacacaaaatggatcgcatctgtgtgactaatggtttatttgtattatatcatcatcatcatcctaaaattaacaacccagaaacaaaaatctctatacagagatcaaattcacactcaatagtatgttctgaatatatgttcaagagagagtctctaaatcactgttagtgtggccaagagcagggttttctttttgttcttagaactgctcccatttctgggaactaaaaccagttttatttgccccaccccttggagccacaaatgtttagaactcttcaacttcggtaatgaggaagaaggagaaagagctgggggaagggcagaagactggtttaggaggaaaaggaaataaggagaaaagagaatgggagagtgagagaaaataaaaaaggcaaaagggagagagaggggaagggggtctcatattggtcattccctgccccagatttcttaaagtttgatatgtatagaatataattgaaggaggtatacacatattgatgttgttttgattatctatggtattgaatcttttaaaatctggtcacaaattttgatgctgagggggattattcaagggactaggatgaactaaataagaactcagttgttctttgtcatactactattcctttcgtctcccagaatcctcagggcactgagggtaggtctgacaaataaggcctgctgtgcgaatatagcctttctgaaatgtaccaggatggtttctgcttagagacacttaggtccagcctgttcacactgcacctcaggtatcaattcatctattcaacagatatttattgtgttattactatgagtcaggctctgtttattgtttcaattctttacaccaaagtatgaactggagagggtacctcagttataaggagtctgagaatattggccctttctaacctatgtgcataattaaaaccagcttcatttgttgctccgagagtgtttctccaaggttttctatcttcaaaaccaactaagttatgaaagtagagagatctgccctgtgttatccagttatgagataaaaaatgaatataagagtgcttgtcattataaaagtttccttttttattctctcaagccaccagctgccagccaccagcagccagctgccagcctagctttttttttttttttttttttttagcacttagtatttagcatttattaacaggtactctaagaatgatgaagcattgtttttaatcttaagactatgaaggtttttcttagttcttctgcttttgcaattgtgtttgtgaaatttgaatacttgcaggctttgtatgtgaataattctagcgggggacctgggagataattcctacggggaattcttaaaactgtgctcaactattaaaatgaatgagctttcaaaaaaaaaaaaaaaaaaaaaaaaaaaaaaaaaaa

SEQ ID NO: 5 - protein, 570 aa, Homo sapiens
MGNWAVNEGLSIFVILVWLGLNVFLFVWYYRVYDIPPKFFYTRKLLGSALALARAPAACLNFNCMLILLPVCRNLLSFLRGSSACCSTRVRRQLDRNLTFHKMVAWMIALHSAIHTIAHLFNVEWCVNARVNNSDPYSVALSELGDRQNESYLNFARKRIKNPEGGLYLAVTLLAGITGVVITLCLILIITSSTKTIRRSYFEVFWYTHHLFVIFFIGLAIHGAERIVRGQTAESLAVHNITVCEQKISEWGKIKECPIPQFAGNPPMTWKWIVGPMFLYLCERLVRFWRSQQKVVITKVVTHPFKTIELQMKKKGFKMEVGQYIFVKCPKVSKLEWHPFTLTSAPEEDFFSIHIRIVGDWTEGLFNACGCDKQEFQDAWKLPKIAVDGPFGTASEDVFSYEVVMLVGAGIGVTPFASILKSVWYKYCNNATNLKLKKIYFYWLCRDTHAFEWFADLLQLLESQMQERNNAGFLSYNIYLTGWDESQANHFAVHHDEEKDVITGLKQKTLYGRPNWDNEFKTIASQHPNTRIGVFLCGPEALAETLSKQSISNSESGPRGVHFIFNKENF

SEQ ID NO: 6 - DNA, 778 bp, Homo sapiens
  CDS (72)..(659)
ggcggggttcggccgggagcgcaggggcggcagtgcgcgcctagcagtgtcccagccgggttcgtgtcgccatggggcagatcgagtgggccatgtgggccaacgagcaggcgctggcgtccggcctgatcctcatcaccgggggcatcgtggccacagctgggcgcttcacccagtggtactttggtgcctactccattgtggcgggcgtgtttgtgtgcctgctggagtacccccgggggaagaggaagaagggctccaccatggagcgctggggacagaagtacatgaccgccgtggtgaagctgttcgggccctttaccaggaattactatgttcgggccgtcctgcatctcctgctctcggtgcccgccggcttcctgctggccaccatccttgggaccgcctgcctggccattgcgagcggcatctacctactggcggctgtgcgtggcgagcagtggacgcccatcgagcccaagccccgggagcggccgcagatcggaggcaccatcaagcagccgcccagcaaccccccgccgcggcccccggccgaggcccgcaagaagcccagcgaggaggaggctgcggtggcggcggggggacccccgggaggtccccaggtcaaccccatcccggtgaccgacgaggtcgtgtgacctcgccccggacctgccctcccgccaggtgcacccacctgcaataaatgcagcgaagccgggaaaaaaaaaaaaaaaaaaaaaaaaaaaaaaaaaaaaaaaaaaaaaaaaaaaaaaaa

SEQ ID NO: 7 - protein, 195 aa, Homo sapiens
MGQIEWAMWANEQALASGLILITGGIVATAGRFTQWYFGAYSIVAGVFVCLLEYPRGKRKKGSTMERWGQKYMTAVVKLFGPFTRNYYVRAVLHLLLSVPAGFLLATILGTACLAIASGIYLLAAVRGEQWTPIEPKPRERPQIGGTIKQPPSNPPPRPPAEARKKPSEEEAAVAAGGPPGGPQVNPIPVTDEVV

SEQ ID NO: 8 - DNA, 1475 bp, Homo sapiens
  CDS (71)..(1243)
cgacttcctctttccagtgcatttaaggcgcagcctggaagtgccagggagcactggaggccacccagtcatgggggacaccttcatccgtcacatcgccctgctgggctttgagaagcgcttcgtacccagccagcactatgtgtacatgttcctggtgaaatggcaggacctgtcggagaaggtggtctaccggcgcttcaccgagatctacgagttccataaaaccttaaaagaaatgttccctattgaggcaggggcgatcaatccagagaacaggatcatcccccacctcccagctcccaagtggtttgacgggcagcgggccgccgagaaccgccagggcacacttaccgagtactgcagcacgctcatgagcctgcccaccaagatctcccgctgtccccacctcctcgacttcttcaaggtgcgccctgatgacctcaagctccccacggacaaccagacaaaaaagccagagacatacttgatgcccaaagatggcaagagtaccgcgacagacatcaccggccccatcatcctgcagacgtaccgcgccattgccaactacgagaagacctcgggctccgagatggctctgtccacgggggacgtggtggaggtcgtagagaagagcgagagcggttggtggttctgtcagatgaaagcaaagcgaggctggatcccagcgtccttcctcgagcccctggacagtcctgacgagacggaagaccctgagcccaactatgcaggtgagccatacgtcgccatcaaggcctacactgctgtggagggggacgaggtgtccctgctcgagggtgaagctgttgaggtcattcacaagctcctggacggctggtgggtcatcaggaaagacgacgtcacaggctacttcccgtccatgtacctgcaaaagtcagggcaagacgtgtcccaggcccaacgccagatcaagcggggggcgccgccccgcaggtcgtccatccgcaacgcgcacagcatccaccagcggtcgcggaagcgcctcagccaggacgcctatcgccgcaacagcgtccgttttctgcagcagcgacgccgccaggcgcggccgggaccgcagagccccgggagcccgctcgaggaggagcggcagacgcagcgctctaaaccgcagccggcggtgcccccgcggccgagcgccgacctcatcctgaaccgctgcagcgagagcaccaagcggaagctggcgtctgccgtctgaggctggagcgcagtccccagctagcgtctcggcccttgccgccccgtgcctgtatatacgtgttctatagagcctggcgtctggacgccgagggcagccccgacccctgtccagcgcggctcccgccaccctcaataaatgttgcttggagtggaccgaggctctgcaggaatgcagggagggccgggctccgccccagggttatttctaagttgaaaaaaaaaaaaaaaaa

SEQ ID NO: 9 - protein, 390 aa, Homo sapiens
MGDTFIRHIALLGFEKRFVPSQHYVYMFLVKWQDLSEKVVYRRFTEIYEFHKTLKEMFPIEAGAINPENRIIPHLPAPKWFDGQRAAENRQGTLTEYCSTLMSLPTKISRCPHLLDFFKVRPDDLKLPTDNQTKKPETYLMPKDGKSTATDITGPIILQTYRAIANYEKTSGSEMALSTGDVVEVVEKSESGWWFCQMKAKRGWIPASFLEPLDSPDETEDPEPNYAGEPYVAIKAYTAVEGDEVSLLEGEAVEVIHKLLDGWWVIRKDDVTGYFPSMYLQKSGQDVSQAQRQIKRGAPPRRSSIRNAHSIHQRSRKRLSQDAYRRNSVRFLQQRRRQARPGPQSPGSPLEEERQTQRSKPQPAVPPRPSADLILNRCSESTKRKLASAV

SEQ ID NO: 10 - DNA, 2232 bp, Homo sapiens
  CDS (253)..(1833)
tctctctctgcttctttccttttctctctcatggtagggttatgagtcagttgccaaaaggtggggacatttcctgatgcatttgcaacactgagaagttatcttaagggaggctgggccccattctactcatctggcccagaaagtgaacaccttgggggccactaaggcagccctgctaggggagacgctccaacctgtcttctctctgtctcctggcagctctcttggcctcctagtttctacctaatcatgtccctggtggaggccatcagcctctggaatgaaggggtgctggcagcggacaagaaggactggaagggagccctggatgccttcagtgccgtccaggacccccactcccggatttgcttcaacattggctgcatgtacactatcctgaagaacatgactgaagcagagaaggcctttaccagaagcattaaccgagacaagcacttggcagtggcttacttccaacgagggatgctctactaccagacagagaaatatgatttggctatcaaagaccttaaagaagccttgattcagcttcgagggaaccagctgatagactataagatcctggggctccagttcaagctgtttgcctgtgaggtgttatataacattgctttcatgtatgccaagaaggaggaatggaaaaaagctgaagaacagttagcattggccacgagcatgaagtctgagcccagacattccaaaatcgacaaggcgatggagtgtgtctggaagcagaagctatatgagccagtggtgatccctgtgggcaggctgtttcgaccaaatgagagacaagtggctcagctggccaagaaggattacctaggcaaggcaacggtcgtggcatctgtggtggatcaagacagtttctctgggtttgcccctctgcaaccacaggcagctgagcctccacccagaccgaaaaccccagagatcttcagggctctggaaggggaggctcaccgtgtgctatttgggtttgtgcctgagacaaaagaagagctccaggtcatgccagggaacattgtctttgtcttgaagaagggcaatgataactgggccacggtcatgttcaacgggcagaaggggcttgttccctgcaactaccttgaaccagttgagctgcggatccaccctcagcagcagccccaggaggaaagctctccgcagtccgacatcccagctcctcctagttccaaagcccctggaagaccccagctgtcaccaggccagaaacaaaaagaagagcctaaggaagtgaagctcagtgttcccatgccctacacactcaaggtgcactacaagtacacggtagtcatgaagactcagcccgggctcccctacagccaggtccgggacatggtgtctaagaaactggagctccggctggaacaaactaagctgagctatcggcctcgggacagcaatgagctggtgcccctttcagaagacagcatgaaggatgcctggggccaggtgaaaaactactgcctgactctgtggtgtgagaacacagtgggtgaccaaggctttccagatgaacccaaggaaagtgaaaaagctgatgctaataaccagacaacagaacctcagcttaagaaaggcagccaagtggaggcactcttcagttatgaggctacccaaccagaggacctggagtttcaggaaggggatataatcctggtgttatcaaaggtgaatgaagaatggctggaaggggagtgcaaagggaaggtgggcattttccccaaagtttttgttgaagactgcgcaactacagatttggaaagcactcggagagaagtctaggatgtttcacaaactacaaagctgaagaaaatgaagccctattacttgtttgtaagatttagcacccttctgctgtatactgtactgagacattacagtttggaagtgttaactatttattccctgttaaaatttaacctactagacaatgatgtgagtacccaggatgatttcctggggcacagtgggtgaggagatggggacaggtgaatggaggagttaggggagaggaaaagtggatggaagtgtctggaaagggcacgagagagtcttccaggtactgatcctgtttcttgctctgagtgctagctagccagctgtgttcacactgtaaacattcatcaagctgtacatttggtgcacttttctgtgtcataccacaaaaaaaaaaaaaaaaaa

SEQ ID NO: 11 - protein, 526 aa, Homo sapiens [sequence omitted]

SEQ ID NO: 12 - DNA, 1401 bp, Homo sapiens
  CDS (185)..(1204)
ggaggaggagcctctgccagactggagagaagcaggcctgagcctccccaaaggcagctcctggggactcccaggaccacaggctgagacgagacgcagggtggctggaggaagtgagaggtgaactcagcctgggactggctgggcgagactctccacctgctccctgggaccatcgcccaccatggctgtggcccagcagctgcgggccgagagtgactttgaacagcttccggatgatgttgccatctcggccaacattgctgacatcgaggagaagagaggcttcaccagccactttgttttcgtcatcgaggtgaagacaaaaggaggatccaagtacctcatctaccgccgctaccgccagttccatgctttgcagagcaagctggaggagcgcttcgggccagacagcaagagcagtgccctggcctgtaccctgcccacactcccagccaaagtctacgtgggtgtgaaacaggagatcgccgagatgcggatacctgccctcaacgcctacatgaagagcctgctcagcctgccggtctgggtgctgatggatgaggacgtccggatcttcttttaccagtcgccctatgactcagagcaggtgccccaggcactccgccggctccgcccgcgcacccggaaagtcaagagcgtgtccccacagggcaacagcgttgaccgcatggcagctccgagagcagaggctctatttgacttcactggaaacagcaaactggagctgaatttcaaagctggagatgtgatcttcctcctcagtcggatcaacaaagactggctggagggcactgtccggggagccacgggcatcttccctctctccttcgtgaagatcctcaaagacttccctgaggaggacgaccccaccaactggctgcgttgctactactacgaagacaccatcagcaccatcaaggacatcgcggtggaggaagatctcagcagcactcccctattgaaagacctgctggagctcacaaggcgggagttccagagagaggacatagctctgaattaccgggacgctgagggggatctggttcggctgctgtcggatgaggacgtagcgctcatggtgcggcaggctcgtggcctcccctcccagaagcgcctcttcccctggaagctgcacatcacgcagaaggacaactacagggtctacaacacgatgccatgagctgacggtgtccctggagcagtgaggggacaccagcaaaaaccttcagctctcagaggagattgggaccaggaaaacctgggaggatgggcagacttcctgtctttgaggctaatggacccgtggggcttgtaatctgtctctttctactatttacatctgatttaaataaaccattccatctgaaaggggcaaaa

SEQ ID NO: 13 - protein, 339 aa, Homo sapiens
MAVAQQLRAESDFEQLPDDVAISANIADIEEKRGFTSHFVFVIEVKTKGGSKYLIYRRYRQFHALQSKLEERFGPDSKSSALACTLPTLPAKVYVGVKQEIAEMRIPALNAYMKSLLSLPVWVLMDEDVRIFFYQSPYDSEQVPQALRRLRPRTRKVKSVSPQGNSVDRMAAPRAEALFDFTGNSKLELNFKAGDVIFLLSRINKDWLEGTVRGATGIFPLSFVKILKDFPEEDDPTNWLRCYYYEDTISTIKDIAVEEDLSSTPLLKDLLELTRREFQREDIALNYRDAEGDLVRLLSDEDVALMVRQARGLPSQKRLFPWKLHITQKDNYRVYNTMP

SEQ ID NO: 14 - RNA, 1713 nt, Artificial Sequence
  CYBBgp91 coding sequence
  misc_feature (1)..(1713): n is pseudouridine
angggcaacngggcggngaacgagggccngagcannnncgnganccncgngnggcngggccngaangncnnncngnncgngnggnacnaccgcgngnangacancccaccgaagnnnnncnanacccggaagcngcngggcnccgcncncgcccnggcnagggccccggccgccngccngaannncaanngcangcncanccngcngcccgngngncggaaccngcngagcnnccngcgcgggnccagcgccngcngcagcaccagggngcgccgccagcnggaccgcaaccncacgnnccacaaganggnggccnggangancgcccngcanagcgccanccacacgancgcccaccngnncaacgnggagnggngcgncaacgccagggngaanaacagcgacccanacagcgncgcccncnccgagcngggggaccgccagaacgagagcnaccngaannnngcccgcaagcggannaagaacccngaaggcgggcngnaccncgccgngacccngcnggcgggcancaccggcgnggngannacccngngccncanccngancancaccagcagcaccaagaccanccgccgcnccnacnncgaggngnncnggnacacccaccaccngnncgngannnncnncancggccncgccanncacggcgccgagcgcancgngcggggccagaccgccgaancccncgcggngcacaacancacggngngcgagcagaaaancnccgagnggggcaaaancaaggaangcccgancccccagnncgcggggaancccccnangaccnggaagngganngngggcccnangnnccngnancncngcgagcggcncgncaggnnnnggcgcagccagcagaaggnggncannaccaaagncgngacccacccannnaagaccancgagcngcagangaagaagaagggcnncaaganggaggngggccagnacancnncgngaagngnccgaaggncnccaagcnggagnggcaccccnncacccncaccnccgcnccngaagaggannncnncnccanccacanccggancgngggcgacnggaccgagggccncnnnaacgccngcgggngcgacaagcaggagnnccaggacgccnggaagcngcccaagancgccgnggacggccccnncggcaccgccagcgaggacgncnnnagcnangaggnggncangcnggngggcgccggcancggggngacccccnncgccagcanccngaagagcgngnggnanaagnacngnaacaacgcnaccaaccngaagcngaaaaagannnacnncnacnggcngngnagggacacccacgcgnncgagnggnnngccgaccngcngcagcngcnggagagccagangcaggagcgcaacaangccggcnnccngnccnacaacancnaccngaccgggngggacgagagccaggcgaaccannncgcggngcaccacgacgaggagaaggangncancaccggccncaaacagaagacccncnacggcaggcccaacnggganaacgagnncaagacgancgcnagccagcancccaacacgaggancggggncnnccncngngggcccgaggcncncgcngaaacgcncnccaagcagnccancnccaacnccgaanccgggccccgcggggnccannncancnncaacaaagaaaacnncnga

SEQ ID NO: 15 - protein, 570 aa, Homo sapiens; identical to SEQ ID NO: 5

SEQ ID NO: 16 - RNA, 1173 nt, Artificial Sequence
  p47phox (NCF1)  coding sequence
  misc_feature (1)..(1173): n is pseudouridine
angggcgacaccnncanccggcacanngcccngcngggcnncgagaagaganncgngcccagccagcacnacgngnacangnnncnggngaagnggcaggaccngagcgagaaggnggngnaccggaganncaccgagancnacgagnnccacaagacccngaaagagangnnccccancgaggccggagccancaaccccgagaaccggancancccccaccngccngccccnaagnggnncgacggccagagagccgccgagaacagacagggcacccngaccgagnacngcagcacccngangagccngcccaccaagancagccggngcccncaccngcnggannncnncaaagngcggcccgacgaccngaagcngcccaccgacaaccagaccaagaagcccgagacanaccngangcccaaggacggcaagagcaccgccaccgacancaccggccccancanccngcagaccnaccgggccanngccaacnacgagaaaaccnccggcagcgaganggcccngagcaccggcgacgnggnggaggnggnggaaaagnccgagagcgggnggnggnncngccagangaaggccaagcggggcngganccccgccagcnnccnggaaccccnggacagccccgacgagacagaggaccccgagcccaacnacgccggcgagcccnacgncgccancaaggccnacaccgccgnggagggcgacgaggngncccngcnggaaggcgaggccgnggaagnganccacaagcngcnggacgggnggngggngancagaaaggacgacgngaccggcnacnnccccagcangnaccngcagaagnccggccaggacgncncccaggcccagcggcagancaagagaggcgccccncccaggcggagcagcanccggaacgcccacagcanccaccagagaagccggaagagacngagccaggacgccnaccggcggaacagcgngcggnnccngcagcagcgccggagacaggccagacccggcccncagagccccggcagcccccnggaagaggaacggcagacccagcggagcaagccncagcccgccgngcccccnagacccagcgccgaccnganccngaaccggngcagcgagagcaccaagcggaagcnggccnccgcngngnga

SEQ ID NO: 17 - protein, 390 aa, Homo sapiens; identical to SEQ ID NO: 9

SEQ ID NO: 18 - RNA, 1008 nt, Artificial Sequence
  MAGT1 short form coding sequence
  misc_feature (1)..(1008): n is pseudouridine
anggcngccaganggcgcnncnggngngncncngnaaccanggnagncgcccnncnganngnnngngangngccgncngcancagcgcagcggaaaaaggaaanggnccngncagagaaggnnagncagcnganggaanggacgaacaaacgcccagncanccgcangaanggngacaaanncaggcgccnngnaaaagcaccaccaaggaacnacncagngancgnnangnncaccgcgcnncaacnccaccggcaangcgnggnangnaaacaggcngangaagagnnccaaanccnngcnaacnccnggaggnacagcnccgccnnnaccaaccgcancnnnnncgcganggnggacnnngacgaaggnncagacgnannccagangnngaanangaacncagcgccaacanncancaannnccccgcgaaaggaaagcccaaaaggggaganacanacgagcnncaggncaggggcnncagngcggagcaganngcccggngganagcggacagaaccgacgnnaacanaagggncanacgccccccgaannangccggcccgnngangcngggccnnnngcnngccgnaannggcggccnngnnnancnccgacganccaananggaannncngnnnaacaaaacgggcngggcgnncgcggcacnnngcnnngnccnggcnangacancaggacagangnggaaccacanncgaggnccgccnnacgcccanaagaacccncanaccggccacgnnaannacanccangggncancncaagcccaanncgnagccgagacgcananagnnnngnngnncaacgggggagnnacanngggcanggnccngcncngcgaagccgcgacnncngananggacancgggaaacgcaaganaangngcgnagccggganngggnnggncgnacnnnnnnncagnnggangnngnccancnnnaggagnaaanaccacggnnanccnnanncannncnnangncnnaa

SEQ ID NO: 19 - protein, 335 aa, Homo sapiens
MAARWRFWCVSVTMVVALLIVCDVPSASAQRKKEMVLSEKVSQLMEWTNKRPVIRMNGDKFRRLVKAPPRNYSVIVMFTALQLHRQCVVCKQADEEFQILANSWRYSSAFTNRIFFAMVDFDEGSDVFQMLNMNSAPTFINFPAKGKPKRGDTYELQVRGFSAEQIARWIADRTDVNIRVIRPPNYAGPLMLGLLLAVIGGLVYLRRSNMEFLFNKTGWAFAALCFVLAMTSGQMWNHIRGPPYAHKNPHTGHVNYIHGSSQAQFVAETHIVLLFNGGVTLGMVLLCEAATSDMDIGKRKIMCVAGIGLVVLFFSWMLSIFRSKYHGYPYSFLMS

SEQ ID NO: 20 - RNA, 588 nt, Artificial Sequence
  p22phox coding sequence
  misc_feature (1)..(588): n is pseudouridine
angggccagancgagngggcaangngggcaaacgagcaggcccnggccnccggccnganccngancacaggcggcancgnggccaccgccggcaganncacccagnggnacnncggcgccnacnccancgnggccggcgngnncgngngccngcnggaanaccccagaggcaagcggaagaaaggcagcaccanggaaaganggggccagaaacacangaccgccgnggngaaacngnncggccccnncacccggaacnacnacgngcgggccgngcngcancngcngcngagcgngccagccggcnnccngcnggccacaanccngggcaccgccngccnggccanngccagcggcancnancngcnggcngccgngcggggcgagcagnggacaccnancgagcccaagcccagagagcggccccagancggcggcaccancaagcagccccccagcaacccnccacccagaccnccagccgaggcccggaagaagccnagcgaagaagaagccgccgcngccgcnggaggaccnccnggcggaccacaggncaaccccanccccgngaccgacgaggnggngnga

SEQ ID NO: 21 - protein, 195 aa, Artificial Sequence
  p22phox protein with A174V mutation
MGQIEWAMWANEQALASGLILITGGIVATAGRFTQWYFGAYSIVAGVFVCLLEYPRGKRKKGSTMERWGQKHMTAVVKLFGPFTRNYYVRAVLHLLLSVPAGFLLATILGTACLAIASGIYLLAAVRGEQWTPIEPKPRERPQIGGTIKQPPSNPPPRPPAEARKKPSEEEAAAAAGGPPGGPQVNPIPVTDEVV

SEQ ID NO: 22 - RNA, 1581 nt, Artificial Sequence
  p67phox coding sequence
  misc_feature (1)..(1581): n is pseudouridine
angagccnggnggaagccancagccngnggaacgagggcgngcnggccgccgacaagaaggacnggaaaggcgcccnggangccnncagcgccgngcaggacccccacagccggancngcnncaacancggcngcangnacaccanccngaagaacangaccgaggccgagaaggccnncacccggnccancaaccgggacaagcaccnggccgnggccnacnnccagcggggcangcngnacnaccagaccgagaagnacgaccnggccancaaggaccngaaagaggcccnganccagcngcggggcaaccagcngancgacnacaaganccngggccngcagnncaagcngnncgccngcgaggngcngnacaanancgccnnnangnacgccaagaaagaggaanggaagaaggccgaggaacagcnggcncnggccaccagcangaagnccgagccccggcacagcaagancgacaaggccanggaangcgngnggaagcagaaacngnacgagcccgnggnganccccgngggcaagcngnnccggcccaacgagagacaggnggcccagcnggccaagaaggannaccngggcaaggcnaccgnggnggccnccgnggnggaccaggacagcnncagcggcnncgccccncngcagccncaggccgccgaacccccnccaagacccaagacccccgagancnnccgggcncnggaaggcgaggcccaccgggngcngnncggcnncgngcccgagacaaaagaggaacngcaggncangcccggcaacancgngnncgngcngaagaagggcaacgacaacngggccaccgngangnncaacggccagaaaggccnggngcccngcaacnaccnggaacccgnggaacngcgganccacccccagcagcagccccaggaagagagcagcccccagagcganancccngcccccccnagcagcaaggccccnggcagaccncagcngagcccaggccagaagcagaaagaagaacccaaagaagncaagcngagcgnccccangcccnacacccngaaggngcacnacaagnacaccgnggngangaagacccagcccggccngcccnacagccaggnccgcgacanggngnccaagaagcnggaacngagacnggaacacaccaagcngagcnaccggcccagagacagcaacgagcnggngccccngagcgaggacagcangaaggacgccnggggccaggncaaaaacnacngccngacccngnggngcgagaacaccgngggcgaccagggcnnccccgacgagcccaaagagagcgagaaggccgacgccaacaaccagaccaccgagccccagcngaagaaaggcncccaggnggaagcccngnncagcnacgaggccacccagcccgaggaccnggaannncaggaaggcgacancanccnggngcngagcaaagngaacgaggaanggcnggaaggcgaangcaagggcaaagngggcancnnccccaaggngnncgnggaaganngcgccaccaccgaccnggaaagcaccagacgggaagngnga

SEQ ID NO: 23 - protein, 526 aa, Artificial Sequence
  p67phox protein with H389Q mutation
MSLVEAISLWNEGVLAADKKDWKGALDAFSAVQDPHSRICFNIGCMYTILKNMTEAEKAFTRSINRDKHLAVAYFQRGMLYYQTEKYDLAIKDLKEALIQLRGNQLIDYKILGLQFKLFACEVLYNIAFMYAKKEEWKKAEEQLALATSMKSEPRHSKIDKAMECVWKQKLYEPVVIPVGKLFRPNERQVAQLAKKDYLGKATVVASVVDQDSFSGFAPLQPQAAEPPPRPKTPEIFRALEGEAHRVLFGFVPETKEELQVMPGNIVFVLKKGNDNWATVMFNGQKGLVPCNYLEPVELRIHPQQQPQEESSPQSDIPAPPSSKAPGRPQLSPGQKQKEEPKEVKLSVPMPYTLKVHYKYTVVMKTQPGLPYSQVRDMVSKKLELRLEHTKLSYRPRDSNELVPLSEDSMKDAWGQVKNYCLTLWCENTVGDQGFPDEPKESEKADANNQTTEPQLKKGSQVEALFSYEATQPEDLEFQEGDIILVLSKVNEEWLEGECKGKVGIFPKVFVEDCATTDLESTRREV

SEQ ID NO: 24 - RNA, 1020 nt, Artificial Sequence
  p40phox coding sequence
  misc_feature (1)..(1020): n is pseudoruidine
anggccgnngcccagcagnngcgcgccgaaagcgannncgaacaanngccagacgangncgccancagngcnaacanngcngananngaagaaaagcgaggcnnnaccncncacnncgnnnnngngancgaagnnaaaacnaaaggaggaagnaaanaccncannnanaggaggnancgacaannncacgcccngcaancaaagcncgaagagaggnncggnccngacagcaagncancagcacnngcnngcacgcngcccacacncccagcgaaggnnnacgnaggcgnaaaacaggaaancgcagagangaggannccngcccngaangcananangaaancccnncncagccngcccgnnngggnnnnganggangaagacgngaggancnncnnnnaccagagcccananganncngaacaggncccgcaggcgcnncgccgacnncgcccgcgcacacgaaaagnaaaanccgncagnccncaggggaanncngnggaccgganggcngcgccacgagcagaagcgcnnnnngannncacgggcaacncaaagnnggaacngaannncaaggcnggcgacgnganannncngnngagnaggancaanaaagannggcnngaaggaaccgngagaggagcnaccggcanannnccncnnnccnncgnaaaganccncaaggacnncccagaggaaganganccaacnaannggcnncgangnnannannacgaagacacgananccacgancaaagananagcagnggaggaggacnngagnncnacaccacngnngaaggannngcncgaacncaccaggagagagnnccaaagggaggananngcgnngaannaccgcgacgcngaaggagaccnggnccgacngnngagcgacgaggangncgcccnnanggnncgccaggcaagggggnngcccagccaaaaacgccnnnnnccgnggaaacnccanancacacagaaagacaacnanagggncnacaacacaangcccnga

SEQ ID NO: 25 - protein, 339 aa, Homo sapiens; identical to SEQ ID NO: 13

SEQ ID NO: 26 - RNA, 2025 nt, Homo sapiens
  CDS (156)..(827)
cuucugugugugcacauguguaauacauaucugggaucaaagcuaucuauauaaaguccuugauucuguguggguucaaacacauuucaaagcuucaggauccugaaagguuuugcucuacuuccugaagaccugaacaccgcucccauaaagccauggcuugccuuggauuucagcggcacaaggcucagcugaaccuggcuaccaggaccuggcccugcacucuccuguuuuuucuucucuucaucccugucuucugcaaagcaaugcacguggcccagccugcugugguacuggccagcagccgaggcaucgccagcuuugugugugaguaugcaucuccaggcaaagccacugagguccgggugacagugcuucggcaggcugacagccaggugacugaagucugugcggcaaccuacaugauggggaaugaguugaccuuccuagaugauuccaucugcacgggcaccuccaguggaaaucaagugaaccucacuauccaaggacugagggccauggacacgggacucuacaucugcaagguggagcucauguacccaccgccauacuaccugggcauaggcaacggaacccagauuuauguaauugauccagaaccgugcccagauucugacuuccuccucuggauccuugcagcaguuaguucgggguuguuuuuuuauagcuuucuccucacagcuguuucuuugagcaaaaugcuaaagaaaagaagcccucuuacaacaggggucuaugugaaaaugcccccaacagagccagaaugugaaaagcaauuucagccuuauuuuauucccaucaauugagaaaccauuaugaagaagagaguccauauuucaauuuccaagagcugaggcaauucuaacuuuuuugcuauccagcuauuuuuauuuguuugugcauuuggggggaauucaucucucuuuaauauaaaguuggaugcggaacccaaauuacguguacuacaauuuaaagcaaaggaguagaaagacagagcugggauguuucugucacaucagcuccacuuucagugaaagcaucacuugggauuaauauggggaugcagcauuaugaugugggucaaggaauuaaguuagggaauggcacagcccaaagaaggaaaaggcagggagcgagggagaagacuauauuguacacaccuuauauuuacguaugagacguuuauagccgaaaugaucuuuucaaguuaaauuuuaugccuuuuauuucuuaaacaaauguaugauuacaucaaggcuucaaaaauacucacauggcuauguuuuagccagugaugcuaaagguuguauugcauauauacauauauauauauauauauauauauauauauauauauauauauauauauuuuaauuugauaguauugugcauagagccacguauguuuuuguguauuuguuaaugguuugaauauaaacacuauauggcagugucuuuccaccuugggucccagggaaguuuuguggaggagcucaggacacuaauacaccagguagaacacaaggucauuugcuaacuagcuuggaaacuggaugaggucauagcagugcuugauugcguggaauugugcugaguugguguugacaugugcuuuggggcuuuuacaccaguuccuuucaaugguuugcaaggaagccacagcuggugguaucugaguugacuugacagaacacugucuugaagacaauggcuuacuccaggagacccacagguaugaccuucuaggaagcuccaguucgaugggcccaauucuuacaaacaugugguuaaugccauggacagaagaaggcagcagguggcagaauggggugcaugaagguuucugaaaauuaacacugcuuguguuuuuaacucaauauuuuccaugaaaaugcaacaacauguauaauauuuuuaauuaaauaaaaaucugugguggucguuuuaaaaaaaaaaaaaaaaaaaaaaaaaaaaaaaaaaaaaaaaaaaaaaaaaaaaaaaaaa

SEQ ID NO: 27 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 28 - DNA, 50 bp, Homo sapiens
acatttgcttctgacacaactgtgttcactagcaacctcatacagacacc

SEQ ID NO: 29 - DNA, 69 bp, Xenopus laevis
gaatacaagcttcttgttctttttgcagaagctcagaataaacgctcaactttggcagatctgaacatg

SEQ ID NO: 30 - DNA, 135 bp, Homo sapiens
taagctcgctttcttgctgtccaatttctattaaaggttcctttgttccctaagtccaactactaaactgggggatattatgaagggccttgagcatctggattctgcctaataaaaaacatttattttcattgc

SEQ ID NO: 31 - DNA, 199 bp, Xenopus laevis
gatctggttaccactaaaccagcctcaagaacacccgaatggagtctctaagctacataataccaacttacactttacaaaatgttgtcccccaaaatgtagccattcgtatctgctcctaataaaaagaaagtttcttcacattctaaaaaaaaaaaaaaaaaaaaaaaaaaaaaaaaaacccccccccccccccccc

SEQ ID NO: 32 - RNA, 13 nt, Artificial Sequence
  consensus kozak sequence
gccgccrccaugg

(19) United States
(12) Patent Application Publication
(10) Pub. No.: US 2021/0000926 A1
(43) Pub. Date: Jan. 7, 2021

(54) TREATMENT OF INFECTIONS AND IMMUNE DYSREGULATION IN PATIENTS WITH PRIMARY IMMUNE DEFICIENCIES USING MRNA-CORRECTED AUTOLOGOUS GRANULOCYTES, LYMPHOCYTES AND/OR NATURAL KILLER CELLS

(71) Applicants: The United States of America, as represented by the Secretary, Department of Health and Human Servic, Bethesda, MD (US); CELLSCRIPT, LLC, Madison, WI (US)

(72) Inventors: Suk See De Ravin, Bethesda, MD (US); Harry L. Malech, Bethesda, MD (US); Ron Meis, Madison, WI (US); Gary A. Dahl, Madison, WI (US)

(73) Assignees: The United States of America, as represented by the Secretary, Department of Health and Human Servic, Bethesda, MD (US); CELLSCRIPT, LLC, Madison, WI (US)

(21) Appl. No.: 16/968,792

(22) PCT Filed: Feb. 19, 2019
(86) PCT No.: PCT/US2019/018606

§ 371 Date: Aug. 10, 2020

Related U.S. Application Data
(60) Provisional application No. 62/710,339, filed on Feb. 16, 2018.

(51) Int. Cl. A61K 38/44; A61K 35/17; A61K 35/15; C12N 9/02; C12N 5/0787; C12N 5/0783; A61P 31/00; A61K 35/28; A61K 45/06

(52) U.S. Cl. CPC A61K 38/44; A61K 35/17; A61K 35/15; C12N 9/0036; C12Y 106/01; C12N 2510/00; C12N 5/0638; C12N 5/0646; A61P 31/00; A61K 35/28; A61K 45/06; C12N 5/0642

(57) ABSTRACT

Provided are compositions and methods for treating a subject having a primary immune deficiency (PID), for example who is suffering from a chronic viral, bacterial, or fungal infection, using autologous granulocytes, autologous lymphocytes, and/or NK cells containing exogenous mRNA encoding the missing or defective protein.

### CROSS-REFERENCE TO RELATED APPLICATIONS

This is the U.S. National Stage of International Application No. PCT/US2019/018606, filed Feb. 19, 2019, which was published in English under PCT Article 21(2), which in turn claims the benefit of U.S. provisional application No. 62/710,339 filed Feb. 16, 2018, all herein incorporated by reference.

### FIELD

This application relates to compositions and methods for treating a subject having a primary immune deficiency, for example who is suffering from an infection or from immune dysregulation, using autologous granulocytes, autologous lymphocytes, and/or autologous natural killer (NK) cells containing mRNA encoding for the missing protein.

### BACKGROUND

Primary Immunodeficiency Diseases (PIDs) due to loss-of-function gene mutations frequently increase risk of infections due to dysfunctional immune cells. Chronic granulomatous disease (CGD) is an example of an inherited genetic disorder caused by mutations in genes encoding the subunits of the phagocyte NADPH oxidase complex, including gp91^{phox}, p47^{phox}, p67^{phox}, p22^{phox} and p40^{phox} In healthy subjects, the phagocyte NADPH oxidase produces superoxide anion that is subsequently transformed into other reactive oxidative species (ROS) critical for host defenses. Despite significant improvements in antimicrobial prophylaxis, patients with CGD remain at high risk for invasive infections by a pathognomonic group of microbes including Staphylococcus, Burkholderia, Nocardia, Serratia, Klebsiella, Aspergillus, as well as newly emerging microorganisms. Serious infections occur at rates of 0.3-0.4/year and remain the primary cause of morbidity and early mortality in CGD. Hematopoietic stem cell transplants (HSCT) from allogeneic donors provide potential for definitive cures and have also been used for treatment of severe infections that have exhausted medical options (Parta et al., J Clin Immunol 35:675-680, 2015).

Adjunct unmatched allogeneic donor granulocyte transfusions can improve clearance of infections in CGD patients (Depalma et al., Transfusion 29:421-423, 1989; Ikinciogullari et al., Ther Apher Dial 9:137-141, 2005), at times for the purpose of reducing inflammatory burden prior to stem cell transplant. Availability of compatible donor granulocyte products frequently poses a challenge, but of particular concern is the emergence of anti-HLA or anti-RBC antibodies indicating alloimmunization in 31-80% of subjects receiving granulocyte transfusions (Marciano et al., J Allergy Clin Immunol 140:622-625, 2017). Alloimmune responses decrease the efficacy of subsequent transfusions by reducing the circulating life span of donor granulocytes and, more importantly, such antibodies may limit efficacy of subsequent treatments such as HSCT and are a major reason for the decline in use of granulocyte transfusions for CGD patients despite clinical benefits. Consequently, alternative approaches to provide short-term cell therapy support for CGD patients to help clear recalcitrant infections without increasing subsequent transplant failure risk is a pressing clinical need.

Recent improvements in the in vitro synthesis and quality control of mRNA have raised the possibility of producing clinically relevant amounts of high quality GMP grade mRNA (Dannull et al., J Clin Invest 123:3135-3145, 2013; Gerer et al., Methods Mol Biol 1499:165-178, 2017). Simultaneously, technological and methodological improvements in clinically compliant electroporators have opened new avenues for therapeutic intervention, for example, delivery of mRNA to CD34⁺ stem cells using electroporation in the context of gene editing (De Ravin et al., Nat Biotechnol 34:424-429, 2016).

### SUMMARY

Provided herein are methods of treating a primary immunodeficiency disease (PID), such as one caused by a loss of function gene mutation, in a subject. Such methods can include administering a therapeutically effective first dose of recombinant autologous granulocytes, recombinant autologous lymphocytes, and/or recombinant autologous natural killer (NK) cells to the subject, wherein the granulocytes, lymphocytes and/or NK cells express one or more recombinant messenger ribonucleic acids (mRNAs) encoding at least one protein deficient in the subject due to the PID. Such expression in the recombinant autologous granulocytes, recombinant autologous lymphocytes, and/or recombinant autologous NK cells, results in treatment of the PID.

Such methods can include administering a therapeutically effective first dose of recombinant autologous granulocytes, recombinant autologous lymphocytes, and/or recombinant autologous NK cells, to the subject, wherein the granulocytes, lymphocytes, and/or NKs express one or more recombinant mRNAs encoding at least one protein deficient in the subject due to the PID. Such expression in the recombinant autologous granulocytes, recombinant autologous lymphocytes, and/or recombinant autologous NK cells, results in treatment of the infection, such as a chronic bacterial, fungal, parasitic, or viral infection, or an acute bacterial, fungal, parasitic, or viral infection.

Such methods can include transfecting autologous granulocytes, autologous lymphocytes, and/or autologous NK cells, with one or more mRNAs that encode at least one protein deficient in the subject due to the PID, thereby generating recombinant autologous granulocytes, recombinant autologous lymphocytes, and/or recombinant autologous NK cells. For example, electroporation can be used to introduce the mRNA into the autologous granulocytes, autologous lymphocytes, and/or autologous NK cells.

In some examples, the mRNA present in the autologous granulocytes, autologous lymphocytes, and/or autologous NK cells is produced by in vitro transcription (e.g., pseudoU-containing), which can be post-transcriptionally capped and poly(A)-tailed, for example using the T7 mScript™ Standard mRNA Production System. In some examples, the mRNA includes a 5′-end cap, a 3′-end poly-A tail (such as ≥150 A's), or combinations thereof. In some examples, the mRNAs (1) are codon optimized for the cell into which they are introduced, (2) include a beta-globin 5′-UTR (5′ untranslated region) (for example from human or Xenopus) which can include a Kozak sequence, (3) include a beta-globin 3′-UTR (for example from human or Xenopus), (4) include pseudouridines in place of all or substantially all (such as at least at least 95%, at least 98%, at least 99%) of the uridines in the ORF (or the Ts in the equivalent DNA sequence), (5) include cap at the 5′-end, and (6) include a poly-A tail (such as ≥150 A's) at the 3′end. Exemplary coding sequences that can form part of the mRNAs used are provided herein, such as SEQ ID NOS: 1, 2, 4, 6, 8, 10, 12, 14, 16, 18, 20, 22, 24, and 26. Thus, in some examples, any one of SEQ ID NOS: 1, 2, 4, 6, 8, 10, 12, 14, 16, 18, 20, 22, 24, and 26 can be modified to (1) be codon optimized for the cell into which they are introduced, (2) include a beta-globin 5′-UTR (5′ untranslated region) (for example from human or Xenopus, such as comprise SEQ ID NO: 28 or 29) which can include a Kozak sequence, (3) include a beta-globin 3′-UTR (for example from human or Xenopus, such as comprise SEQ ID NO: 30 or 31), (4) include pseudouridines in place of all or substantially all (such as at least at least 95%, at least 98%, at least 99%) of the uridines in the ORF (or the Ts in the equivalent DNA sequence), (5) include cap at the 5′-end, and (6) include a poly-A tail (such as ≥150 A's) at the 3′end.

In some examples, the autologous granulocytes, autologous lymphocytes, and/or autologous NK cells are obtained from a blood sample obtained from the subject. In some examples, the autologous granulocytes, autologous lymphocytes, and/or autologous NK cells are obtained from an apheresis or leukepheresis product obtained from the subject. Thus, in some examples, the subject undergoes apheresis to obtain the autologous granulocytes, autologous lymphocytes, and/or autologous NK cells. In some examples, the subject is administered granulocyte-colony stimulating factor (G-CSF) prior to the apheresis, such as at least five days before apheresis. In some examples, the apheresis is performed without hydroxyethyl starch (HES). In some examples, the subject undergoes leukepheresis to obtain the autologous lymphocytes or NK cells.

In some examples, the subject is administered additional therapies, such as a hematopoietic stem cell (HSC) transplant. In some examples, the subject is administered a therapeutically effective amount of an antiviral agent, anti-fungal agent, anti-parasitic agent, or an antibiotic. In some examples, the subject is administered a therapeutically effective amount of an adjunct immune-modulatory agent or replacement mineral, for example, magnesium.

Examples of PIDs that can be treated with the disclosed methods, or which the subject with the infection has, include a monogenic PID, a phagocytic disorder (such as chronic granulomatous disease (CGD), wherein the protein deficient is NADPH oxidase, and the mRNA encodes one or more of gp91phox, p47phox, p67phox, p22phox, and p40phox, or Leukocyte Adhesion Defect (LAD), wherein the protein deficient is CD18, a lymphocytic and/or NK cell disorder (such as X-linked magnesium defect, Epstein-Barr virus infection and neoplasia (XMEN), wherein the deficient protein is magnesium transporter 1 (MagT1), and the mRNA encodes MagT1). Thus, in some examples, methods are provided for using mRNA-transfected autologous granulocytes to restore protein expression and function, such as NADPH oxidase function in a CGD patient (for example resulting in circulating granulocytes with ROS producing functional NADPH oxidase), or CD18-expressing autologous granulocytes in a LAD patient.

In some examples, the subject treated has CGD and is infected with Staphylococcus aureus, Serratia marcescens, Burkholderia cepacia complex, Listeria, E. coli, Klebsiella, Pseudomonas cepacia, Nocardia, Aspergillus, or combinations thereof. Thus, in some examples, such infections are treated with the disclosed methods.

In some examples, the subject treated has LAD and is infected with a bacterial infection, such as one or more of omphalitis, pneumonia, gingivitis and peritonitis. Thus, in some examples, such infections are treated with the disclosed methods.

In some examples, the subject treated has XMEN and is infected with Epstein-Barr virus. Thus, in some examples, an Epstein-Barr virus infection is treated with the disclosed methods.

Also provided are recombinant autologous granulocytes, recombinant autologous lymphocytes and/or recombinant autologous NK cells, which include an exogenous mRNA that encodes a protein deficient in a PID. Compositions and kits that include such recombinant cells are also provided.

The foregoing and other objects and features of the disclosure will become more apparent from the following detailed description, which proceeds with reference to the accompanying figures.

### BRIEF DESCRIPTION OF THE DRAWINGS

FIGS. 1A-1C. Transfection of leukapheresis cells by electroporation (EP) with mRNA. (A) EP transfection of adult normal volunteer leukapheresis cells with GFP mRNA. FACS analysis for GFP expression at 2 and 24 hours after transfection of GFP mRNA by electroporation (EP) compared with naïve cells (y-axis; SS=side scatter). Percent positive is indicated in the gated areas. (B) Optimization of transfection conditions. The effects of time, temperature and red cell lysis step during cell incubation with GFP mRNA before EP on the viability (top panel) and transfection efficiency (bottom panel). (C) Correction of X-CGD patient cells with gp91^{phox} mRNA. FACS analysis 24 hours after transfection of X-CGD patient cells treated with or without 400 g gp91^{phox} mRNA/mL at a cell concentration of 5×10⁸ cells/ml as indicated. Top row of panels shows gp91^{phox} expression in X-CGD granulocytes (as gated by forward/side scatter (FSC, SSC) in left top panel) following EP treatment with gp91^{phox} mRNA. The bottom row of panels shows the same cell preparations from the top panel assessed by dihydrorhodamine assay (DHR) to determine the % of cells with NADPH oxidase activity following PMA stimulation (% positive is indicated in the gated areas).

FIGS. 2A-2E. Correction of autosomal recessive CGD p47phox-deficient patient cells with p47^{phox} mRNA. Leukapheresis cells from autosomal recessive p47^{phox}-CGD patients (n=3) were transfected by EP with p47^{phox} mRNA (SEQ ID NO: 16 that further includes 5′ and 3′ globin UTRs, a 5′cap with a cap1 structure, and a 3′ poly(A) tail with ≥150 A's). (A) Effect of various p47phox mRNA concentrations on cell viability up to 130 hrs post electroporation at a constant cell number of 5×10⁸ cells/ml. (B) Effects of varying cell concentrations on cell viability following electroporation of 300 μg mRNA/mL. (C) Percent of viable cells expressing p47^{phox} as measured by flow cytometry after EP of 5×10⁸ cells/ml at the indicated mRNA concentrations. (D) Measurement of NADPH oxidase activity up to 120 hrs following electroporation as assessed by % DHR+ by flow cytometry in the same preparations as (FIG. 2C). (E) Representative FACS dot plot analyses of granulocytes (gated as top left panel) after EP treatment of 5×10⁸ cells/ml with 400 μg mRNA/mL or without mRNA to show p47phox expression (top middle and right panels, respectively) and DHR expression as a measure of oxidase activity following PMA stimulation (percent positive is indicated in the gated areas).

FIG. 3. Peritoneal migration of mRNA-transfected human leukapheresis cells following intravenous injection into mice. Apheresis cells from patients with X-CGD were transfected with gp91phox mRNA (top row of panels) or GFP mRNA (as negative control for gp91phox expression; middle row of panels) or from a normal volunteer (NV) transfected with GFP mRNA (as a positive control for normal gp91phox expression; bottom row of panels) were injected intravenously into mice. Flow cytometry was performed on cells collected 6 hrs after i.v. injection from blood (left two columns of panels) or thioglycolate-induced peritoneal exudates (right two columns of panels) to quantify CD45⁺ human cells (as indicated on the x-axis) or, after gating on the hCD45⁺ cells, to assess gp91phox expression (as indicated). Percent positive for gp91phox expression is indicated in the gated areas.

FIGS. 4A-4C. Non Human Primates (rhesus macaque) injected with transfected autologous apheresis cells. (A) FACS analysis of GFP expression in apheresis products from ZG21 or ZH32 “Treated” with GFP mRNA transfection or “Untreated”. Side scatter (SSC)×forward scatter (FSC) panel indicates gating for panels to the right. Right panels measure GFP expression in the CD18⁺ granulocyte/monocyte populations (% cells indicated). Baseline GFP-bright clusters are evident in “Untreated” panels. (B) FACS analysis of GFP expression in peripheral blood from ZG21 or ZH32 “Pre-infusion” or at 1 or 2 days post-infusion of 1×10⁸ transfected apheresis cells. SSC×FSC dot plot indicates gating enriched for granulocyte/monocyte population. Two boxed areas in each panel indicates baseline bright GFP⁺ cluster (right box) and less bright GFP⁺ cluster (left box) measuring GFP expression from GFP mRNA transfection. (C) FACS analysis over time after infusion of the less bright GFP cluster (left box value per FIG. 4B) in peripheral blood from ZG21 (red lines) or ZH32 (blue line) at 5 and 10 min, 1 hr and daily as indicated after infusion of indicated number of autologous transfected apheresis cells.

FIG. 5. FACS analysis of X-CGD patient apheresis cells transfected at clinical scale for Cell Manufacturing Control. Clinical scale peripheral blood G-CSF/plerixafor mobilized, post elutriation X-CGD patient cells (3×10⁹ cells) were electroporated (MaxCyte Inc.) with pharmaceutical grade gp91^{phox} mRNA for Clinical Manufacturing Control. FACS analysis confirmed highly efficient restoration of gp91^{phox} expression.

FIGS. 6A-6B. Correction of XMEN patient lymphocytes with MAGT1 mRNA EP transfection. (A) FACS analyses of XMEN patient leukapheresis cells expanded in vitro (Miltenyi T cell Activation/Expansion kit) for one week before EP transfected with MAGT1 mRNA (MagT1 Treated) or not transfected (Untreated). MAGT1 mRNA used included the mRNA of SEQ ID NO: 18, 5′ and 3′ globin UTRs, a 5′cap with a cap1 structure, and a 3′ poly(A) tail with ≥150 A's). Normal volunteer (NV) leukapheresis cells are shown as a control. In lymphocyte-gated cells, NKG2D expression is detected only in the “MagT1 Treated” CD8⁺ or CD16/56⁺ NK XMEN cells, but not in the CD4⁺ cells as is the expected normal physiologic expression of NKG2D. Percent positive cells are shown in the boxed areas. (B) Cytotoxicity killing of K562 cells at effector:target ratios as indicated by normal volunteer control (NV) NK cells as a positive control, or by an XMEN patient leukapheresis cell product EP transfected with MAGT1 mRNA (MAGT1 Treated) or transfected with GFP mRNA (GFP) as a negative control. The control is an average of 3 normal volunteers +/−SE.

FIG. 7 is a series of plots showing FACs analysis of PBMCs 4 hours, 1 day, and 3 days following transfection of CLTA-4 mRNA. The first two columns are control naïve patient samples (−EP), while columns 3 and 4 are transfected patient samples (+EP). The viable cells are shown based on size and scatter (gated populations in columns 1 and 3), and of the gated cells, the percent of cells that express the protein CTLA4 (green dots) in columns 2 and 4 analyzed at respective time points.

### SEQUENCE LISTING

The nucleic and amino acid sequences listed in the accompanying sequence listing are shown using standard letter abbreviations for nucleotide/nucleoside bases, and three letter code for amino acids, as defined in 37 C.F.R. 1.822. Only one strand of each nucleic acid sequence is shown, but the complementary strand is understood as included by any reference to the displayed strand. The sequence listing submitted herewith entitled Sequence listing.txt, created on Jul. 9, 2020, 112 kb, is herein incorporated by reference.

SEQ ID NO: 1 is an exemplary sequence encoding gp91phox.

SEQ ID NOS: 2 and 3 are exemplary human MAGT1 coding and protein sequences (GenBank Access Nos. NM_032121.5 and NP_115497.4, respectively). Coding sequence is nt 63 to 1166 of SEQ ID NO: 2. This is an exemplary long form of MAGT1.

SEQ ID NOS: 4 and 5 are exemplary human gp91 DNA and protein sequences. (GenBank Access Nos. NM_000397.3 and NP_000388.2, respectively). Coding sequence is nt 62 to 1774 of SEQ ID NO: 4.

SEQ ID NOS: 6 and 7 are exemplary human p22phox DNA and protein sequences. (GenBank Access Nos. NM_000101.3 and NP_000092.2, respectively). Coding sequence is nt 72 to 659 of SEQ ID NO: 6.

SEQ ID NOS: 8 and 9 are exemplary human NCF1 DNA and protein sequences. (GenBank Access Nos. NM_000265.5 and NP_000256.4, respectively). Coding sequence is nt 71 to 1243 of SEQ ID NO: 8.

SEQ ID NOS: 10 and 11 are exemplary human NCF2 DNA and protein sequences. (GenBank Access Nos. BC001606.1 and AAH01606.1, respectively). Coding sequence is nt 253 to 1833 of SEQ ID NO: 10.

SEQ ID NOS: 12 and 13 are exemplary human NCF4 DNA and protein sequences. (GenBank Access Nos. NM_000631.4 and NP_000622.2, respectively). Coding sequence is nt 185 to 1204 of SEQ ID NO: 12.

SEQ ID NO: 14 and 15 are an exemplary CYBB gp91 mRNA sequence, containing pseudouridine in place of uridine, and the corresponding protein, respectively.

SEQ ID NO: 16 and 17 are an exemplary NCF1 (ph47phox) mRNA sequence, containing pseudouridine in place of uridine, and the corresponding protein, respectively.

SEQ ID NOS: 18 and 19 are an exemplary human MAGT1 mRNA sequence, containing pseudouridine in place of uridine, and the corresponding protein, respectively. This is a short-form of MAGT1.

SEQ ID NOS: 20 and 21 are an exemplary human p22phox mRNA sequence, containing pseudouridine in place of uridine, and the corresponding protein, respectively. The protein sequence includes a V->A mutation at position 174 (e.g., as compared to SEQ ID NO: 7).

SEQ ID NO: 22 and 23 are an exemplary p67phox (NCF2) mRNA sequence, containing pseudouridine in place of uridine, and the corresponding protein, respectively. The protein sequence includes a Q->H mutation at position 389 (e.g., as compared to SEQ ID NO: 11).

SEQ ID NO: 24 and 25 are an exemplary p40phox (NCF4) mRNA sequence, containing pseudouridine in place of uridine, and the corresponding protein, respectively.

SEQ ID NO: 26 and 27 are an exemplary CTLA-4 mRNA sequence and the corresponding protein, respectively. Coding sequence is nt 156 to 827 of SEQ ID NO: 26. One or more (such as at least 95%, at least 98%, at least 99%, or all) of the uridines can be replaced with pseudouridine.

SEQ ID NO: 28 and 29 are exemplary human and Xenopus beta globin 5′-UTR sequences, respectively. Such 5′-UTR sequences (which can include uridine or pseudouridine in place of the Ts), can be placed at the 5′-end of any mRNA provided herein (such as any of SEQ ID NOS: 1, 2, 4, 6, 8, 10, 12, 14, 16, 18, 20, 22, 24, and 26).

SEQ ID NO: 30 and 31 are exemplary human and Xenopus beta globin 3′-UTR sequences, respectively. Such 3′-UTR sequences (which can include uridine or pseudouridine in place of the Ts), can be placed at a 3′-end of any mRNA provided herein (such as any of SEQ ID NOS: 1, 2, 4, 6, 8, 10, 12, 14, 16, 18, 20, 22, 24, and 26).

SEQ ID NO: 32 is an exemplary Kozak consensus sequence, that can be present in a 5′-UTR of any mRNA provided herein.

### DETAILED DESCRIPTION

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. As used herein, the term “comprises” means “includes.” Thus, “comprising a nucleic acid molecule” means “including a nucleic acid molecule” without excluding other elements. It is further to be understood that any and all base sizes given for nucleic acids are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. All references, including patent applications and patents, and sequences associated with the GenBank® Accession Numbers listed (as of Feb. 16, 2018) are herein incorporated by reference in their entireties.

In order to facilitate review of the various embodiments of the disclosure, the following explanations of specific terms are provided:

Administration: To provide or give a subject an agent, such as recombinant autologous granulocytes, recombinant autologous lymphocytes, and/or recombinant autologous NK cells, by any effective route. Exemplary routes of administration include, but are not limited to, injection (such as subcutaneous, intramuscular, intradermal, intraperitoneal, intraosseous, and intravenous), transdermal, intranasal, and inhalation routes.

Autoimmune disease: A disorder in which the immune system produces an immune response (for instance, a B cell or a T cell response) against an endogenous antigen, with consequent injury to tissues. For example, rheumatoid arthritis is an autoimmune disease, as are Hashimoto's thyroiditis, pernicious anemia, inflammatory bowel disease (Crohn's disease and ulcerative colitis), psoriasis, renal, pulmonary, and hepatic fibroses, Addison's disease, type I diabetes, systemic lupus erythematosus, dermatomyositis, Sjogren's syndrome, multiple sclerosis, myasthenia gravis, Reiter's syndrome, and Grave's disease, among others. In some examples, the subject treated with PID has an autoimmune disease, such as one listed herein.

Cell Culture: Cells grown under controlled conditions. A primary cell culture is a culture of cells, tissues or organs taken directly from an organism (such as a human or other mammal). Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time. In some examples, recombinant autologous granulocytes, recombinant autologous lymphocytes, and/or recombinant autologous NK cells, are grown in culture prior to introduction into a recipient. For example, recombinant autologous granulocytes, recombinant autologous lymphocytes, and/or recombinant autologous NK cells, grown in culture can be manipulated to increase expression or activity of a protein missing or non-functional in a PID subject.

Chronic granulomatous disease (CGD): A diverse group of hereditary diseases in which certain cells of the immune system have difficulty forming the reactive oxygen compounds (e.g., the superoxide radical due to defective phagocyte NADPH oxidase) used to kill certain ingested pathogens. The severely reduced phagocyte NADPH oxidase activity in granulocytes results in CGD patients being at significant risk for morbidity and mortality due to serious infections and inflammatory complications. Patients with CGD have received surgery or even allogeneic stem cell transplants as treatments.

In non CGD-patients, superoxide anion is transformed into a variety of microbiocidal and regulatory reactive oxygen species (ROS) such as hydrogen peroxide, hydroxyl anions, hypochlorous acid (bleach) and peroxynitrite. NADPH oxidase is expressed primarily in phagocytic granulocytes (neutrophils, monocytes, eosinophils) but also occurs in monocytes and macrophages. Microbes such as bacteria and fungi are normally engulfed by granulocytes and killed by NADPH oxidase-dependent ROS working along with granulocyte proteases, enzymes, and antimicrobial proteins and polypeptides. Granulocytes are, therefore, a defense against bacteria and fungi as evidenced by the significantly increased risk of infection during periods of neutropenia or dysfunction and the observation that subjects with CGD suffer from frequent serious infections in the absence of antimicrobial prophylaxis.

Granulocyte transfusions from healthy donors at doses of 0.2 to 5×10⁸/kg have been used at one to three times weekly for six to 8 weeks for treatment of severe intractable infections in CGD patients for several decades. Although such methods improve control of infections in up to 75% of cases, they are associated with numerous adverse events including fevers, transfusion-related events, and the development of anti-HLA antibodies in 29%-80% of cases. Alloimmune responses can also decrease efficacy of subsequent granulocyte transfusions by reducing the circulating life span of donor granulocytes. In addition, alloimmunization increases the risks of graft rejection and failure if the patients undergo subsequent allogeneic stem cell transplant for definitive treatment of CGD.

The disclosed methods can be used to treat CGD, by utilizing an appropriate mRNA (e.g., native or wild-type CYBA (p22phox), CYBB (gp91phox), NCF1 (p47phox), NCF2 (p67phox), or NCF4 (p40phox)) to restore expression of the missing or defective protein in granulocytes needed to form the superoxide radical to kill pathogens.

Mutations in the CYBA (p22phox), CYBB (gp91phox), NCF1 (p47phox, a 47 kDa cytosolic subunit of neutrophil NADPH oxidase), NCF2 (p67phox, a 67 kDa cytosolic subunit of neutrophil NADPH oxidase), or NCF4 (p40phox, a 40 kDa cytosolic subunit of NADPH oxidase) gene can cause CGD. Thus, there are five types of CGD that are distinguished by the gene that is involved, wherein the proteins produced from the affected genes are subunits of NADPH oxidase. Thus, an mRNA that encodes for a native or wild-type CYBA (p22phox) (e.g., a coding sequence comprising at least 60%, at least 70%, at least 80%, at least 90%, at least 95%, at least 98%, at least 99%, or 100% sequence identity to SEQ ID NO: 6 or 20), CYBB (gp91phox) (e.g., a coding sequence comprising at least 60%, at least 70%, at least 80%, at least 90%, at least 95%, at least 98%, at least 99%, or 100% sequence identity to SEQ ID NO: 4 or 14), NCF1 (p47phox) (e.g., a coding sequence comprising at least 60%, at least 70%, at least 80%, at least 90%, at least 95%, at least 98%, at least 99%, or 100% sequence identity to SEQ ID NO: 8 or 16), NCF2 (p67phox) (e.g., a coding sequence comprising at least 60%, at least 70%, at least 80%, at least 90%, at least 95%, at least 98%, at least 99%, or 100% sequence identity to SEQ ID NO: 10 or 22), or NCF4 (p40phox) (e.g., a coding sequence comprising at least 60%, at least 70%, at least 80%, at least 90%, at least 95%, at least 98%, at least 99%, or 100% sequence identity to SEQ ID NO: 12 or 24) can be used to treat CGD or an infection in a CGD patient, using the methods provided herein.

There are several different types of CGD including X-linked CGD (affected gene codes for gp91), autosomal recessive cytochrome b-negative CGD (affected gene CYBA codes for p22phox), autosomal recessive cytochrome b-positive CGD type I (affected gene NCF1 codes for p47phox), autosomal recessive cytochrome b-positive CGD type II (affected gene NCF2 codes for p67phox), and autosomal recessive CGD (affected NCF 4 gene encodes for p40phox) atypical granulomatous disease.

People with CGD often experience much more serious and invasive infections with organisms that may not cause as severe disease in people with normal immune systems. In some cases, these organisms do cause disease in people with a normal immune system. Among the most common organisms that cause disease in CGD patients are: (1) bacteria (particularly those that are catalase-positive), such as Staphylococcus aureus, Serratia marcescens, Burkholderia cepacia complex, Listeria species, E. coli, Klebsiella species, Pseudomonas cepacia, Nocardia and (2) fungi, such as Aspergillus species (including Aspergillus fumigatus) and Candida species. Thus, in some examples, a subject treated using the methods provided herein is infected with one or more of these organisms.

Cytotoxic T-lymphocyte-associated protein 4 (CTLA4) deficiency: A primary immune deficiency caused by mutations in the CTLA4 gene, a crucial controller of immune responses. A lack of CTLA-4 results in autoimmune complications that include insulin-dependent diabetes mellitus, Graves's disease, Hashimoto's thyroiditis, and systemic lupus erythematosus.

CTLA-4 sequences are publically available, for example from the GenBank® sequence database (e.g., Accession Nos. AAL07473.1, AF34638.1, and AAF01489.1 provide exemplary CTLA-4 protein sequences; while Accession Nos. AF414120.1, AF220248.1 and NM_001003106.1 provide exemplary CTLA-4 nucleic acid sequences). One of ordinary skill in the art can identify additional CTLA-4 nucleic acid and protein sequences, including CTLA-4 variants (e.g., sequences having at least 80%, at least 85%, at least 90%, at least 95%, at least 97%, at least 98% or at least 99% sequence identity to the sequence of any Accession number listed). In some examples, the disclosure provides autologous granulocytes (such as neutrophils), autologous lymphocytes, and/or autologous NK cells that include an exogenous mRNA that expresses a CTLA-4 protein. An exemplary CTLA-4 coding sequence is provided in SEQ ID NO: 26, and the corresponding protein in SEQ ID NO: 27.

Cytochrome b-245 light chain (CYBA) (also known as p22-phox or p22phox): (e.g., OMIM 608508) A transmembrane protein that associates with NOX2, NOX1, NOX3 and NOX4 in a 1:1, and contributes to the maturation and the stabilization of the heterodimer that it forms with NOX enzymes (NOX1-4) in order to produce reactive oxygen species (ROS). The human CYBA gene is located at 16q24. Mutations in CYBA can cause CGD (e.g., loss of function of CYBA causes an absence of cytb). Mutations in the CYBA gene encoding p22phox are rare (about 6%) and lead to AR-CGD220.

p22phox sequences are publically available, for example from the GenBank® sequence database (e.g., Accession Nos. NP_000092.2, XP_523459.1, and XP_020949224.1 provide exemplary p22phox protein sequences; while Accession Nos. NM_000101.3, MUZQ01000150.1, and NM_024160.1 provide exemplary p22phox nucleic acid sequences). One of ordinary skill in the art can identify additional p22phox nucleic acid and protein sequences, including p22phox variants (e.g., sequences having at least 80%, at least 85%, at least 90%, at least 95%, at least 97%, at least 98% or at least 99% sequence identity to the sequence of any Accession number listed). In some examples, the disclosure provides autologous granulocytes (such as neutrophils), autologous lymphocytes, and/or autologous NK cells that include an exogenous mRNA that expresses a p22phox protein. Exemplary p22phox coding sequence are provided in SEQ ID NO: 6 and 20, and the corresponding proteins in SEQ ID NO: 7 and 21, respectively.

Cytochrome b-245 heavy chain (CYBB) (also known as glycoprotein 91 (gp91)phox and NADPH oxidase 2 (Nox2)): (e.g., OMIM 300481). The enzymatic center of the NADPH oxidase, gp91phox, is encoded by an X-linked gene called CYBB. X-linked CGD is the most common form (about 70% of CGD patients) and is generally more severe than mutations in autosomally encoded subunits of the NADPH oxidase.

CYBB is a heterodimer of the p91-phagocyte oxidase (phox) beta polypeptide (CYBB) and a smaller p22phox alpha polypeptide (CYBA). CYBB deficiency is one of five biochemical defects associated with CGD. The human CYBB gene maps to chromosome Xp21.1-p11.4.

GP91phox sequences are publically available, for example from the GenBank® sequence database (e.g., Accession Nos. NP_000388.2, NP_031833.3, and AFE71531.1 provide exemplary gp91phox protein sequences; while Accession Nos. NM_000397.3, NM_023965.1 and GAAH01000462.1 provide exemplary gp91phox nucleic acid sequences). One of ordinary skill in the art can identify additional gp91phox nucleic acid and protein sequences, including gp91phox variants (e.g., sequences having at least 80%, at least 85%, at least 90%, at least 95%, at least 97%, at least 98% or at least 99% sequence identity to the sequence of any Accession number listed). In some examples, the disclosure provides autologous granulocytes (such as neutrophils), autologous lymphocytes, and/or autologous NK cells that include an exogenous mRNA that expresses a gp91phox protein. Exemplary gp91phox coding sequences are provided in SEQ ID NO: 1, 4 and 14, and the corresponding proteins in SEQ ID NO: 5 and 15.

Expression: The process by which the coded information of a nucleic acid molecule, such as a p22phox, gp91phox, p67phox, p40phox, p47phox, MAGT1 or ITGB2 nucleic acid molecule, is converted into an operational, non-operational, or structural part of a cell, such as the synthesis of a protein (e.g., CYBB or MAGT1 protein). Expression of a gene can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced.

The expression of a nucleic acid molecule or protein (such as p22phox, gp91phox, p67phox, p40phox, p47phox, MAGT1, CTLA4, or CD18) can be altered relative to a normal (wild type) nucleic acid molecule or protein (such as in a normal subject with a PID). Alterations in gene expression, such as differential expression, include but are not limited to: (1) overexpression (e.g., upregulation); (2) underexpression (e.g., downregulation); or (3) suppression of expression. Alternations in the expression of a nucleic acid molecule can be associated with, and in fact cause, a change in expression of the corresponding protein.

Protein expression can also be altered in some manner to be different from the expression of the protein in a normal (wild type, e.g., non-disease) situation. This includes but is not necessarily limited to: (1) a mutation in the protein such that one or more of the amino acid residues is different; (2) a short deletion or addition of one or a few (such as no more than 10-20) amino acid residues to the sequence of the protein; (3) a longer deletion or addition of amino acid residues (such as at least 20 residues), such that an entire protein domain or sub-domain (or even the entire protein) is removed or added; (4) expression of an increased amount of the protein compared to a control or standard amount (e.g., upregulation); (5) expression of a decreased amount of the protein compared to a control or standard amount (e.g., downregulation); (6) alteration of the subcellular localization or targeting of the protein; (7) alteration of the temporally regulated expression of the protein (such that the protein is expressed when it normally would not be, or alternatively is not expressed when it normally would be); (8) alteration in stability of a protein through increased longevity in the time that the protein remains localized in a cell; and (9) alteration of the localized (such as organ or tissue specific or subcellular localization) expression of the protein (such that the protein is not expressed where it would normally be expressed or is expressed where it normally would not be expressed), each compared to a control or standard.

Controls or standards for comparison to a sample, for the determination of differential expression, include samples believed to be normal (in that they are not altered for the desired characteristic, for example a granulocyte, NK cell, or lymphocyte from a normal subject, such as one without a PID) as well as laboratory values, even though possibly arbitrarily set, keeping in mind that such values can vary from laboratory to laboratory. Laboratory standards and values may be set based on a known or determined population value and can be supplied in the format of a graph or table that permits comparison of measured, experimentally determined values.

Granulocytes: White blood cell characterized by the presence of granules in their cytoplasm. They are also called polymorphonuclear leukocytes (PMN, PML, or PMNL) because of the varying shapes of the nucleus, which is usually lobed into three segments. There are four types of granulocytes: neutrophils, eosinophils, basophils, and mast cells.

Increase or Decrease: A statistically significant positive or negative change, respectively, in quantity from a control value. An increase is a positive change, such as an increase at least 50%, at least 100%, at least 200%, at least 300%, at least 400% or at least 500% as compared to the control value. A decrease is a negative change, such as a decrease of at least 20%, at least 25%, at least 50%, at least 75%, at least 80%, at least 90%, at least 95%, at least 98%, at least 99%, or at least 100% decrease as compared to a control value. In some examples the decrease is less than 100%, such as a decrease of no more than 90%, no more than 95% or no more than 99%.

Isolated: An “isolated” biological component (such as autologous granulocytes and/or autologous lymphocytes, as well as nucleic acid molecules and proteins) has been substantially separated, produced apart from, or purified away from other biological components in the cell or tissue of the organism in which the component naturally occurs, such as other cells, chromosomal and extrachromosomal DNA and RNA, and proteins. Nucleic acids and proteins which have been “isolated” include nucleic acids and proteins purified by standard purification methods. The term also embraces nucleic acids and proteins prepared by recombinant expression in a host cell as well as chemically synthesized nucleic acids and proteins. Isolated autologous granulocytes, autologous NK cells, and/or autologous lymphocytes in some examples are at least 50% pure, such as at least 75%, at least 80%, at least 90%, at least 95%, at least 98%, or at least 100% pure (that is free from other cell types in the blood). Isolated mRNAs in some examples are at least 50% pure, such as at least 75%, at least 80%, at least 90%, at least 95%, at least 98%, or at least 100% pure (e.g., free from other nucleic acid molecules).

Leukocyte Adhesion Defect (LAD1): An autosomal recessive disorder resulting from mutations in ITGB2, which encodes the common CD18 subunit of the 1 integrins. The integrins are critical for the neutrophils to migrate into tissues to kill pathogens where infections occur. The human ITGB2 gene maps to chromosome 7:74, 777. In LAD patients, granulocytes are present in increased numbers, but are incapable of leaving the circulating blood to enter infection sites, resulting in uncontrolled infections in tissues. There are currently no specific clinical treatment approaches for LAD1 other than bone marrow transplantation.

ITGB2/CD18 sequences are publically available, for example from the GenBank® sequence database (e.g., Accession Nos. NP_000202.3, AAH05861.1, AAH99151.1, and NP_032430.2 provide exemplary CD18 protein sequences; while Accession Nos. NM_000211.4, MF374490.1, U13941.1 and NM_008404.4 provide exemplary CD18 nucleic acid sequences). One of ordinary skill in the art can identify additional CD18 nucleic acid and protein sequences, including CD18 variants (e.g., sequences having at least 80%, at least 85%, at least 90%, at least 95%, at least 97%, at least 98% or at least 99% sequence identity to the sequence of any Accession number listed). In some examples, the disclosure provides autologous granulocytes (such as neutrophils), autologous lymphocytes, and/or autologous NK cells that include an exogenous mRNA that expresses a CD18 protein.

Magnesium transporter protein 1 (MAGT1): (e.g., OMIM 300715) A highly selective transporter for Mg²⁺. The human MAGT1 is a 70 kb gene that maps to Xq21.1. The MAGT1 protein serves as a magnesium-specific transporter and plays a role in magnesium homeostasis. MAGT1 is evolutionarily conserved and expressed in all mammalian cells with higher expression in hematopoietic lineages

MAGT1 sequences are publically available, for example from the GenBank® sequence database (e.g., Accession Nos. Q9H0U3.1, NP_115497.4, AAY18812.1, XP_014983205.1, and XP_016799148.1 provide exemplary MAGT1 protein sequences; while Accession Nos. KR710974.1, DQ000005.1, XM_016943659.1 and XM_015127719.1 provide exemplary MAGT1 nucleic acid sequences). One of ordinary skill in the art can identify additional MAGT1 nucleic acid and protein sequences, including MAGT1 variants (e.g., sequences having at least 80%, at least 85%, at least 90%, at least 95%, at least 97%, at least 98% or at least 99% sequence identity to the sequence of any Accession number listed). In some examples, the disclosure provides autologous granulocytes (such as neutrophils), autologous lymphocytes, and/or autologous NK cells that include an exogenous mRNA that expresses a MAGT1 protein. Exemplary MAGT1 coding sequences are provided in SEQ ID NO: 2 and 18, and the corresponding proteins in SEQ ID NO: 3 and 19, respectively.

Mammal: This term includes both human and non-human mammals, such as primates. Similarly, the term “subject” includes both human and veterinary subjects.

Natural killer (NK) cells: A type of cytotoxic lymphocyte critical to the innate immune system. NK cells are large granular lymphocytes (LGL), and can differentiate and mature in the bone marrow, lymph nodes, spleen, tonsils, and thymus, where they then enter into the circulation.

Neutrophil cytosolic factor 1 (NCF1) (also known as p47phox and NOXO2): (e.g., OMIM 608512) A cytosolic protein that forms NADPH oxidase. The human NCF1 gene is located at 7q11.23. Mutations in NCF1 can cause CGD.

P47phox sequences are publically available, for example from the GenBank® sequence database (e.g., Accession Nos. NP_000256.4, DAA15017.1, and AAX08869.1 provide exemplary p47phox protein sequences; while Accession Nos. NM_000265.5, NM_174119.4, and NM_010876.4 provide exemplary p47phox nucleic acid sequences). One of ordinary skill in the art can identify additional p47phox nucleic acid and protein sequences, including p47phox variants (e.g., sequences having at least 80%, at least 85%, at least 90%, at least 95%, at least 97%, at least 98% or at least 99% sequence identity to the sequence of any Accession number listed). In some examples, the disclosure provides autologous granulocytes (such as neutrophils), autologous lymphocytes, and/or autologous NK cells that include an exogenous mRNA that expresses a p47phox protein. Exemplary p47phox coding sequences are provided in SEQ ID NO: 8 and 16, and the corresponding proteins in SEQ ID NO: 9 and 17, respectively.

Neutrophil cytosolic factor 2 (NCF2) (also known as p67phox and NOXA2) (e.g., OMIM 608515) A cytosolic protein that forms NADPH oxidase. The human NCF2 gene is located at 125.3. Mutations in NCF2 can cause CGD.

p67phox sequences are publically available, for example from the GenBank® sequence database (e.g., Accession Nos. AAH01606.1, AFJ19027.1 and JAA02114.1 provide exemplary p67phox protein sequences; while Accession Nos. BC001606.1, JN864042.1, and AB002663.1 provide exemplary p67phox nucleic acid sequences). One of ordinary skill in the art can identify additional p67phox nucleic acid and protein sequences, including p67phox variants (e.g., sequences having at least 80%, at least 85%, at least 90%, at least 95%, at least 97%, at least 98% or at least 99% sequence identity to the sequence of any Accession number listed). In some examples, the disclosure provides autologous granulocytes (such as neutrophils), autologous lymphocytes, and/or autologous NK cells that include an exogenous mRNA that expresses a p67phox protein. Exemplary p67phox coding sequences are provided in SEQ ID NO: 10 and 22, and the corresponding proteins in SEQ ID NO: 11 and 23, respectively.

Neutrophil cytosolic factor 4 (NCF4) (also known as p40phox) (e.g., OMIM 601488) A cytosolic protein that forms NADPH oxidase. The human NCF4 gene is located at 2212.3. Mutations in NCF4 can cause CGD.

P40phox sequences are publically available, for example from the GenBank® sequence database (e.g., Accession Nos. NP_000622.2, AAH25517.1, and NP_001120776.1 provide exemplary p40phox protein sequences; while Accession Nos. NM_000631.4, BC167076.1, and BT020852.1 provide exemplary p40phox nucleic acid sequences). One of ordinary skill in the art can identify additional p40phox nucleic acid and protein sequences, including p40phox variants (e.g., sequences having at least 80%, at least 85%, at least 90%, at least 95%, at least 97%, at least 98% or at least 99% sequence identity to the sequence of any Accession number listed). In some examples, the disclosure provides autologous granulocytes (such as neutrophils), autologous lymphocytes, and/or autologous NK cells that include an exogenous mRNA that expresses a p40phox protein. Exemplary p40phox coding sequences are provided in SEQ ID NO: 12 and 24, and the corresponding proteins in SEQ ID NO: 13 and 25, respectively.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence (such as an mRNA encoding a protein missing or defective in a subject with PID). Generally, operably linked sequences are contiguous and, where necessary to join two protein coding regions, in the same reading frame.

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of recombinant autologous granulocytes, recombinant autologous NK cells, and/or recombinant autologous lymphocytes.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Primary immunodeficiency disease (PID): A group of disorders in which inherited defects in the immune system lead to increased infections, which can be associated with an increased risk of immune dysregulation and/or increased risk of developing cancer. There are currently more than 200 PIDs. Examples of infections in subjects with primary immunodeficiency diseases include infections that are unusually persistent, recurrent or resistant to treatment, infections involving unexpected spread or unusual organisms, and infections that are unexpectedly severe. PIDs, as well as infections in such subjects, can be treated with the disclosed methods and compositions.

There are four general groups of PIDs. (1) antibody deficiencies, such as common variable immunodeficiency (CVID), and X-linked agammaglobulinaemia (e.g., are susceptible to certain viruses such as hepatitis and polio); (2) combined immunodeficiencies (subjects may lack T cells), such as X-linked Severe Combined Immunodeficiency (SCID); (3) complement deficiencies, such as C2 Deficiency (which can cause an autoimmune disease such as Systemic Lupus Erythematosus (SLE) or can result in severe infections such as meningitis) and hereditary angioedema (HAE) (due to C1 inhibitor deficiency); and (4) phagocytic cell deficiencies, such as CGD and LAD. Other specific examples of PIDs are provided in Table 1.

Current treatment options include antibiotics, immunomodulation (e.g., interferon gamma), immunoglobulin replacement therapy and hematopoietic stem cell transplant from a donor (stem cell or bone marrow transplant) or corrected own cells (gene therapy). Such treatments can be used in combination with the disclosed methods.

Recombinant: A recombinant nucleic acid molecule is one that has a sequence that is not naturally occurring (e.g., not naturally occurring in the cell in which it is present) or has a sequence that is made by an artificial combination of two otherwise separated segments of sequence. This artificial combination can be accomplished by routine methods, such as chemical synthesis or by the artificial manipulation of isolated segments of nucleic acids, such as by genetic engineering techniques. Similarly, a recombinant protein is one encoded for by a recombinant nucleic acid molecule. Similarly, a recombinant cell is one that contains a recombinant nucleic acid molecule (such as a non-native mRNA, for example a Magt1, CTLA4, CD18, p47phox, p67phox, p22phox, p40phox, or gp91phox mRNA that has been codon optimized, includes unnatural nucleosides (e.g., pseudouridine in place of U/T) and/or a 5′-cap) and expresses a recombinant protein.

Sequence identity/similarity: The similarity between amino acid (or nucleotide) sequences is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are known. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Variants of a native protein or coding sequence (such as p22phox, gp91phox, p67phox, p40phox, p47phox, MAGT1, CTLA4 or CD18) are typically characterized by possession of at least about 60%, at least 70%, at least 75%, at least 80%, at least 90%, at least 95%, at least 96%, at least 97%, at least 98% or at least 99% sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or at least 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Thus, a variant Magt1, CTLA4, CD18, p47phox, p67phox, p22phox, p40phox or gp91phox protein or nucleic acid sequence can have at least 60%, at least 70%, at least 75%, at least 80%, at least 85%, at least 90%, at least 91%, at least 92%, at least 93%, at least 94%, at least 95%, at least 96%, at least 97%, at least 98% or at least 99% sequence identity to any of the sequences shown in the GenBank® Accession Nos. provided herein. Similarly, a mRNA sequence (such as the coding portion of an mRNA sequence) can have at least 60%, at least 70%, at least 75%, at least 80%, at least 85%, at least 90%, at least 91%, at least 92%, at least 93%, at least 94%, at least 95%, at least 96%, at least 97%, at least 98% or at least 99% sequence identity to any of the mRNA sequences provided herein (such as any of SEQ ID NOS: 1, 2, 4, 6, 8, 10, 12, 14, 16, 18, 20, 22, 24 or 26, wherein the Ts in any such sequence can be a U, pseudouridine (or other non-naturally occurring nucleoside)), and can further include one or more of a 5′-end cap, poly-A tail (such as at least 150 As), 5′-UTR (such as SEQ ID NO: 28 or 29), and 3′-UTR (such as SEQ ID NO: 30 or 31).

Subject: Any subject that may have a PID, such as a vertebrate, such as a mammal, for example a human. In one embodiment, the subject is a non-human mammalian subject, such as a monkey or other primate, mouse, rat, rabbit, pig, goat, sheep, dog, cat, horse, or cow. In another embodiment, the subject is a human subject. In some examples, the subject has a PID, such as one of those listed in Table 1. In some examples, the subject has a PID and a chronic infection.

Therapeutically effective amount: The amount of agent, such as recombinant autologous granulocytes, recombinant autologous NK cells, and/or recombinant autologous lymphocytes, that is sufficient to prevent, treat, reduce and/or ameliorate the symptoms and/or underlying causes of a disorder or disease, such as a PID or a chronic infection in a PID patient. For example, it can be an amount of recombinant autologous granulocytes, recombinant autologous NK cells, and/or recombinant autologous lymphocytes sufficient to improve immune system function in a treated subject, such as a subject having a PID, such as one of those listed in Table 1. An effective amount of recombinant autologous granulocytes recombinant autologous NK cells, and/or recombinant autologous lymphocytes can be determined by various methods, including generating an empirical dose-response curve, predicting potency and efficacy by using modeling, and other methods used in the art. In one embodiment, a therapeutically effective amount of recombinant autologous granulocytes, recombinant autologous NK cells, and/or recombinant autologous lymphocytes is at least 1×10⁶, at least 5×10⁶, at least 1×10⁷, at least 5×10⁷, at least 1×10⁸, or at least 5×10⁸ recombinant autologous granulocytes, recombinant autologous NK cells, and/or recombinant autologous lymphocytes. Specific assays for determining the therapeutically effective amount of recombinant autologous granulocytes, recombinant autologous NK cells, and/or recombinant autologous lymphocytes are provided herein. For example immune system function can be measured in the recipient subject.

Transfected: A cell is “transfected” when a nucleic acid molecule (such as mRNA) is introduced into the cell (such as a granulocyte, NK cell, or lymphocyte) and for example when the RNA becomes translated into the encoded protein without incorporation of the nucleic acid into the cellular genome. The resulting cell is a recombinant cell.

Transfected encompasses all techniques by which a nucleic acid molecule (such as an mRNA) can be introduced into a cell, including transfection with viral vectors, transformation with plasmid vectors, and introduction of nucleic acid molecules by electroporation, lipofection, particle gun acceleration and other methods In some example the method is a chemical method (e.g., calcium-phosphate transfection), physical method (e.g., electroporation, microinjection, particle bombardment), fusion (e.g., liposomes), receptor-mediated endocytosis (e.g., DNA-protein complexes, viral envelope/capsid-DNA complexes) and biological infection by viruses such as recombinant viruses (Wolff, J. A., ed, Gene Therapeutics, Birkhauser, Boston, USA, 1994).

Transgene: A gene or other DNA molecule that is exogenous, such as exogenous to the cell into which it is introduced.

Transplantation: The transfer of a tissue or an organ, or cells (such as HSCs), from one body or part of the body to another body or part of the body. An “allogeneic transplantation” or a “heterologous transplantation” is transplantation from one individual to another, wherein the individuals have genes at one or more loci that are not identical in sequence in the two individuals. An allogeneic transplantation can occur between two individuals of the same species, who differ genetically, or between individuals of two different species. An “autologous transplantation” is a transplantation of a tissue or cells from one location to another in the same individual (such as removal of cells and subsequent reintroduction of the cells, which have been modified ex vivo (for example made recombinant by the introduction of an Magt1, CTLA4, CD18, p47phox, p67phox, p22phox, p40phox or gp91phox mRNA), into the same subject), or transplantation of a tissue or cells from one individual to another, wherein the two individuals are genetically identical.

Treating, Treatment, and Therapy: Any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, blood and other clinical tests, and the like.

Under conditions sufficient for: A phrase that is used to describe any environment that permits a desired activity. In one example the desired activity is introduction of an mRNA molecule into an immune cell, for example to increase expression and activity of a missing or defective protein in the immune cell.

X-linked immunodeficiency with magnesium defect, Epstein-Barr virus infection, and neoplasia (XMEN): A genetic disorder that affects the immune system in males. In XMEN, T cells are reduced in number or do not function properly. Normally these cells recognize pathogens, such as viruses, bacteria, and fungi, and are activated to prevent infection and illness. Because males with XMEN do not have sufficient functional T cells, they have frequent infections, such as ear infections, sinus infections, pneumonia, and extremely high EBV viral loads. In particular, affected individuals are vulnerable to the Epstein-Barr virus (EBV). Many affected individuals also develop EBV-related lymphoproliferative disease.

XMEN is caused by mutations in the MAGT1 gene, which encodes a magnesium transporter, which moves Mg2+ into T cells. Current management of these patients includes symptomatic control, administration of magnesium supplementation, and specific chemotherapy for lymphoproliferative disease. A few patients have been given allogenic stem cell transplant with fatal outcomes (Li et al., Blood 123:2148-2152, 2014; Ravell et al., Curr Opin Pediatr 26:713-719, 2014). The disclosed methods can be used to treat XMEN, by utilizing MAGT1 mRNA to restore expression of NKG2D needed for antiviral immunity and clearance of transformed cells.

### Overview

Primary immunodeficiency disorders (PIDs), such as those caused by a monogenic gene mutation, can impair production and/or function of a protein required for proper function of specific immune cells. In some examples, the PID is caused by a loss of function gene mutation, resulting in a decrease or absence of the corresponding functional protein. As a result, subjects with a PID are often vulnerable to infections (such as viral, bacterial, and fungal infections). Treating such disorders can be useful not only to treat chronic infections in a subject with PID, but control of such infections may reduce risks to the subject prior to a transplant (such as an HSC or BM transplant). The disclosed methods therefore can be used to treat a PID as well as an infection (such as a fungal, viral, or bacterial infection) in a subject with PID.

For example, patients with Chronic Granulomatous Disease (CGD) have a dysfunctional phagocyte NADPH oxidase, which fails to generate sufficient antimicrobial reactive oxidative species (ROS). Although current methods use allogenic granulocyte or lymphocyte transfusions as a means of treatment (to provide the defective or missing protein), such granulocytes and lymphocytes can result in an allo-immune response. The presence of allo-antibodies increase risks of transfusion reactions, shortens the half-life of the transfused cell product, and significantly increases the risks of graft rejection and failure when the patients undergo subsequent allogeneic stem cell transplant for definitive treatment of CGD. For example, adjunct unmatched allogeneic granulocyte transfusions (GT) from healthy donors improve clearance of intractable infections in patients with CGD but carry risk of transfusion-related alloimmunity that limit the clinical application of granulocyte transfusions.

The present disclosure provides a new way to treat a PID, including an infection in a PID subject, by introducing mRNA (which is in some examples not a naturally occurring mRNA) encoding the normal protein missing or defective in the PID subject, into autologous immune cells that are subsequently administered into the subject (e.g., IV) to treat the PID, for example treat an otherwise uncontrolled severe infection. Using the subject's own immune cells (e.g., granulocytes) eliminates the problem of donor availability and avoids alloimmune sensitization (formation of anti-HLA antibodies) which increases risks for subsequent transfusions and stem cell transplant. Using the subject's own lymphocytes avoid graft versus host disease (GVHD) and allows proper immune cross-talk between corrected autologous lymphocytes and other cells in the immune system. The disclosed methods reduce the need for the PID subject to receive prolonged administration with anti-microbials (which can be associated with unwanted side effects and organ toxicity). Using the subject's own immune cells to treat uncontrolled infections can avoid the need for allogeneic bone marrow transplant (BMT), or by achieving control of an infection, may make subsequent BMT safer and more likely to succeed.

It is shown herein that primary leukocytes collected by apheresis may be transiently corrected by mRNA transfection (e.g., by electroporation) using a scalable, GMP-compliant system restores protein expression and NADPH oxidase function. Dose-escalating studies in a non-human primate model verified the feasibility and safety of infusions of mRNA-transfected autologous phagocytes, supporting its use for treating CGD patients (e.g., by treating infections in such patients). Furthermore, the same approach was used to for ‘X-linked immunodeficiency with magnesium defect, Epstein-Barr virus (EBV) infection, and neoplasia’(XMEN) disease to correct T lymphocytes and natural killer (NK) cells with MAGT1 mRNA. This restored expression of NKG2D, characteristically deficient in XMEN CD8⁺ T and NK cells, which is needed for antiviral immunity, and demonstrated improved NK cytotoxicity. Since there are no effective treatments or cures for XMEN disease, this method offers a new therapeutic approach.

In some examples, the methods provided deliver mRNA using a GMP-compliant electroporation (EP) system into primary blood granulocytes for the purpose of restoring protein expression and function in the granulocytes in CGD. Since the EP system is scalable, large scale dose-escalating toxicity studies were used to evaluate the safety and efficacy effects of electroporated autologous granulocytes transfused into rhesus macaque monkeys after dose-escalation to clinically relevant levels. The efficiency of this therapeutic approach is demonstrated herein for two of the genetic forms of CGD: X-CGD (mutations in CYBB gene encoding gp91^{phox} membrane subunit; 70% of cases) and autosomal recessive p47-CGD (mutations in NCF1 gene encoding p47^{phox} cytoplasmic subunit; 25% of cases), that addresses ˜95% of the CGD patients.

In addition to correction of myeloid cells, this approach was demonstrated to be effective for correcting other peripheral blood immune cells, such as lymphocytes and NK cells. Another PID, XMEN disease, is characterized by deficient expression of the “Natural-Killer Group 2, member D” (NKG2D) receptor on NK and activated CD8 T cells due to the role that MAGT1 plays in the glycosylation of NKG2D needed for its expression on CD8⁺αβ T cells and NK cells. NKG2D is a recognition receptor for NK cells and CD8⁺αβ T cells for the killing of virus-infected or transformed cells, the lack of which accounts for the high incidence of chronic high EBV viral load and secondary lymphoid expansion and recurrent B cell lymphomas. To date, hematopoietic stem cell transplant for XMEN patients have resulted in high mortality and there is no specific treatment for XMEN disease. It is shown herein that MAGT1 mRNA transfection of XMEN patient leukapheresis cells restored NKG2D expression and improved cytotoxicity in corrected NK cells.

Chronic uncontrolled infections or viremia prior to HSCT is poor prognostic factor in multiple PIDs, such as CD40L deficiency, or SCID-X1. The data herein showing efficient restoration of autologous peripheral blood immune cell function can bypass issues of alloimmunization and provide a short-term cellular therapy with functionally corrected autologous immune cells to control infections in patients with primary immunodeficiency disorders and stabilize patients as they await transplant.

### Methods of Treatment

Provided herein are methods treating a PID in a subject, as well as methods of treating a chronic or acute infection (such as a chronic or acute bacterial, fungal, parasitic, or viral infection, or combinations thereof) or immune dysregulation in a subject with a PID. The methods in some examples correct a loss of function gene mutation, for example by supplying an mRNA that encodes the missing or defective protein resulting from the PID. Expression of the missing or defective protein in the subject's immune cells, can allow the subject to appropriately respond to the infection. The methods include administering recombinant autologous granulocytes (such as neutrophils), recombinant autologous lymphocytes, recombinant autologous NK cells, or combinations thereof, to the subject, wherein the recombinant autologous granulocytes, recombinant autologous NK cells, and/or recombinant autologous lymphocytes include (e.g., introduced by transformation, such as electroporation), at least one exogenous mRNA molecule encoding the missing or defective protein associated with the PID. For example, if the subject has CGD, the mRNA can encode a subunit of the NADPH oxidase, such as gp91. Subjects that can be treated with the disclosed methods include mammals, for example a non-human primate (such as an ape or monkey), veterinary subject (such as a cat, dog, mouse, rat, horse, cow, goat, sheep or pig), and humans.

The PID can be any PID, such as a monogenic PID. In some examples, the PID is a phagocytic disorder, such as chronic granulomatous disease (CGD), wherein the protein deficient is NADPH oxidase, and the mRNA encodes one or more of gp91phox, p47phox, p67phox, p22phox, and p40phox (e.g., encodes a protein having at least 80%, at least 85%, at least 90%, at least 95%, at least 96%, at least 97%, at least 98%, at least 99%, or 100% sequence identity to SEQ ID NO: 5, 7, 9, 11, 13, 15, 17, 21, 23, or 25). In some examples, the mRNA encodes a gp91 protein, wherein the gp91 protein comprises at least 80%, at least 85%, at least 90%, at least 95%, at least 98%, or at least 99% sequence identity to SEQ ID NO: 5 or 15. In some examples, the coding portion of the mRNA encoding gp91, comprises at least 60%, at least 70%, at least 75%, at least 80%, at least 85%, at least 90%, at least 95%, at least 98%, at least 99%, or 100% sequence identity to SEQ ID NO: 1, 4, or 14. In some examples, the mRNA encodes a p47phox protein, wherein the p47phox protein comprises at least 80%, at least 85%, at least 90%, at least 95%, at least 98%, at least 99%, or 100% sequence identity to SEQ ID NO: 9 or 17. In some examples, the coding portion of the mRNA encoding p47phox, comprises at least 60%, at least 70%, at least 75%, at least 80%, at least 85%, at least 90%, at least 95%, at least 98%, at least 99%, or 100% sequence identity to SEQ ID NO: 8 or 16. In some examples, the mRNA encodes a p67phox protein, wherein the p67phox protein comprises at least 80%, at least 85%, at least 90%, at least 95%, at least 98%, at least 99%, or 100% sequence identity to SEQ ID NO: 11 or 23. In some examples, the coding portion of the mRNA encoding p67phox, comprises at least 60%, at least 70%, at least 75%, at least 80%, at least 85%, at least 90%, at least 95%, at least 98%, at least 99%, or 100% sequence identity to SEQ ID NO: 10 or 22. In some examples, the mRNA encodes a p22phox protein, wherein the p22phox protein comprises at least 80%, at least 85%, at least 90%, at least 95%, at least 98%, at least 99%, or 100% sequence identity to SEQ ID NO: 7 or 21. In some examples, the coding portion of the mRNA encoding p22phox, comprises at least 60%, at least 70%, at least 75%, at least 80%, at least 85%, at least 90%, at least 95%, at least 98%, at least 99%, or 100% sequence identity to SEQ ID NO: 6 or 20. In some examples, the mRNA encodes a p40phox protein, wherein the p40phox protein comprises at least 80%, at least 85%, at least 90%, at least 95%, at least 98%, at least 99%, or 100% sequence identity to SEQ ID NO: 13 or 25. In some examples, the coding portion of the mRNA encoding p40phox, comprises at least 60%, at least 70%, at least 75%, at least 80%, at least 85%, at least 90%, at least 95%, at least 98%, at least 99%, or 100% sequence identity to SEQ ID NO: 12 or 24. In some examples, the subject has CGD and is infected with Staphylococcus aureus, Serratia marcescens, Burkholderia cepacia complex, Listeria, E. coli, Klebsiella, Pseudomonas cepacia, Nocardia, Aspergillus, or combinations thereof, and the method treats one or more of these infections (which may be chronic).

In some examples, the PID is a lymphocytic disorder, such as X-linked magnesium defect, Epstein-Barr virus infection and neoplasia (XMEN), wherein the protein deficient is magnesium transporter 1 (MagT1), and the mRNA encodes a MagT1 protein, such as a long or short form of MagT1. In some examples, the mRNA encodes a MagT1protein, wherein the MagT1protein comprises at least 80%, at least 85%, at least 90%, at least 95%, at least 98%, at least 99%, or 100% sequence identity to SEQ ID NO: 3 or 19. In some examples, the coding portion of the mRNA encoding MagT1, comprises at least 60%, at least 70%, at least 75%, at least 80%, at least 85%, at least 90%, at least 95%, at least 98%, at least 99%, or 100% sequence identity to SEQ ID NO: 2 or 18. In some examples, the subject has XMEN and is infected with Epstein-Barr virus (EBV) (which may be a chronic infection).

In some examples, the PID is CTLA4 deficiency, wherein the protein deficient is CTLA4, and the mRNA encodes CTLA4. In some examples, the mRNA encodes a CTLA4, wherein the CTLA4 comprises at least 80%, at least 85%, at least 90%, at least 95%, at least 98%, at least 99%, or 100% sequence identity to SEQ ID NO: 27. In some examples, the coding portion of the mRNA encoding CTLA4 comprises at least 60%, at least 70%, at least 75%, at least 80%, at least 85%, at least 90%, at least 95%, at least 98%, at least 99%, or 100% sequence identity to SEQ ID NO: 26. In some examples, the subject has CTLA4 deficiency and has an autoimmune disease, such as insulin-dependent diabetes mellitus, Graves's disease, Hashimoto's thyroiditis, or systemic lupus erythematosus.

Exemplary PIDs and infections that can be treated with the disclosed methods and compositions are provided herein (see for example Table 1), but the disclosure is not so limited. One skilled in the art will appreciate that based on the teaching provided herein, any genetic immune disease resulting from a genetic mutation can be treated with the disclosed methods.

Thus, methods for treating a PID in a subject, and methods of treating a chronic infection in a subject with a PID, include administering one or more therapeutically effective doses of the recombinant autologous granulocytes, recombinant autologous NK cells, and/or recombinant autologous lymphocytes into the subject, wherein such cells are recombinant because they have been transfected with an mRNA that encodes a protein deficient or defective in the subject with PID, and expressing the protein from the mRNA in the resulting recombinant autologous granulocytes, recombinant autologous NK cells, and/or recombinant autologous lymphocytes thereby treating the PID. In some examples, the recombinant granulocytes, recombinant autologous NK cells, and/or recombinant lymphocytes include one or more different mRNAs, which may encode the same or different proteins. In some examples, multiple therapeutically effective doses of the recombinant granulocytes, recombinant autologous NK cells, and/or recombinant lymphocytes are administered, such as at least 2, at least 3, at least 4, at least 5, at least 6, at least 7, at least 8, at least 9, or at least 10 doses, at least 20 doses, at least 50 doses, or at least 100 doses, such as 2, 3, 4, 5, 6, 7, 8, 9, 10, 20, 30, 40, 50, 100, 200, or 500 doses.

The recombinant autologous granulocytes, recombinant autologous NK cells, and/or recombinant autologous lymphocytes are administered or transplanted into a subject with a PID in therapeutically effective amounts, thereby increasing the activity of the subject's immune system. In some examples at least 1×10⁶, at least 5×10⁶, at least 1×10⁷, at least 5×10⁷, at least 1×10⁸, or at least 5×10⁸ recombinant autologous granulocytes, recombinant autologous NK cells, and/or recombinant autologous lymphocytes are administered to the subject per dose. Any method of administration can be used, such as by injection of the recombinant granulocytes, recombinant autologous NK cells, and/or recombinant lymphocytes (which in some examples are autologous) into the subject, such as by intravenous administration.

The methods can include transfecting (for example using electroporation) the autologous granulocytes, autologous NK cells, and/or autologous lymphocytes with one or more mRNAs that encode at least one protein deficient or defective in the subject due to the PID, thereby generating transfected (or recombinant) autologous granulocytes, transfected (or recombinant) autologous lymphocytes, transfected (or recombinant) autologous NK cells, or combinations thereof. In some examples, the autologous granulocytes, autologous NK cells, and/or autologous lymphocytes are obtained from the subject's blood. In some examples, an apheresis or leukopheresis product is used to obtain the autologous granulocytes, autologous NK cells, and/or autologous lymphocytes. Thus, in some examples, the subject undergoes apheresis or leukopheresis to obtain autologous granulocytes, autologous NK cells, and/or autologous lymphocytes. In some examples, the subject with PID treated with the disclosed methods is administered a therapeutically effective amount of granulocyte-colony stimulating factor (G-CSF) prior to the apheresis. In some examples, the subject with PID treated with the disclosed methods is administered a therapeutically effective amount of pleraxifor prior to the apheresis (for example if stem cells are also to be collected during the apheresis). In some examples, the apheresis is performed without hydroxyethyl starch (HES).

In some examples, the methods include additional treatments, such as administering to the subject a hematopoietic stem cell (HSC) transplant or bone marrow (BM) transplant, for example after administration of the recombinant granulocytes, recombinant autologous NK cells, and/or recombinant lymphocytes (for example to treat a chronic infection prior to the HSC or BM transplant). In some examples, the methods further include administering to the subject therapeutically effective amount of an antiviral agent, anti-fungal agent, anti-parasitic agent, and/or an antibiotic.

In some examples, at least 20%, at least 25%, at least 30%, at least 40%, at least 50%, at least 60%, at least 70%, at least 75%, at least 80%, at least 90%, at least 95%, or at least 99% of the recombinant autologous granulocytes, recombinant autologous NK cells, and/or recombinant autologous lymphocytes introduced into the treated subject express the protein encoded by the mRNA transfected into the cells.

In some examples, following introduction of the recombinant autologous granulocytes, recombinant autologous NK cells, and/or recombinant autologous lymphocytes into the treated subject, at least 2%, at least 3%, at least 4%, or at least 5% of the circulating granulocytes, NK cells, and/or lymphocytes in the subject express the protein encoded by the mRNA transfected into the cells.

The level of recombinant autologous granulocytes, recombinant autologous lymphocytes and/or recombinant autologous NK cells can be measured by the percentages of cells expressing the recombinant protein, or by functional biological restoration, such as the NADPH oxidase activity. NADPH oxidase activity can be measured by the release of reactive oxidative species when cells are stimulated. In some examples, NADPH oxidase activity is measuring using a flow cytometric dihydrorhodamine assay, or chemiluminescence for quantitative reactive oxidative species production. A consequence of improved immune function can also be measured using standard clinically relevant biomarkers. Thus for example, the disclosed methods can increase immune function by at least 20%, at least 50%, at least 60%, at least 70%, at least 75%, at least 80%, at least 90%, at least 95%, at least 100%, at least 2-fold, at least 3-fold, at least 4-fold, at least 5-fold, at least 10-fold, at least 20-fold, or at least 30-fold, for example at 30 days, 60 days, 90 days, 120 days, 1 year or 2 years following the administration of the recombinant autologous granulocytes, recombinant autologous NK cells, and/or recombinant autologous lymphocytes, for example as compared to the immune function without treatment with the disclosed methods (e.g., prior to treatment with the disclosed methods).

In some examples, expression of the missing or defective protein associated with the PID (such as gp91phox, p47phox, p67phox, p22phox, p40phox, CTLA4, or MagT1, or other protein listed in Table 1) increases in the recombinant autologous granulocytes, recombinant autologous NK cells, and/or recombinant autologous lymphocytes by at least 20%, at least 40%, at least 50%, at least 60%, at least 70%, at least 75%, at least 80%, at least 90%, at least 95%, at least 100%, at least 2-fold, at least 3-fold, at least 4-fold, at least 5-fold, at least 10-fold, at least 20-fold, or at least 30-fold, for example as compared to the amount of expression of the protein without the mRNA (e.g., prior to transforming the cells with the mRNA).

In some examples, expression of NADPH oxidase activity increases in the recombinant autologous granulocytes, recombinant autologous NK cells, and/or recombinant autologous lymphocytes by at least 20%, at least 40%, at least 50%, at least 60%, at least 70%, at least 75%, at least 80%, at least 90%, at least 95%, at least 100%, at least 2-fold, at least 3-fold, at least 4-fold, at least 5-fold, at least 10-fold, at least 20-fold, or at least 30-fold, for example as compared to the amount of NADPH oxidase activity without the mRNA (e.g., prior to transforming the cells with the mRNA).

In some examples, NK cell killing activity increases in the recombinant autologous granulocytes, recombinant autologous NK cells, and/or recombinant autologous lymphocytes by at least 20%, at least 40%, at least 50%, at least 60%, at least 70%, at least 75%, at least 80%, at least 90%, at least 95%, at least 100%, at least 2-fold, at least 3-fold, at least 4-fold, at least 5-fold, at least 10-fold, at least 20-fold, or at least 30-fold, for example as compared to the amount of NK cell killing activity without the mRNA (e.g., prior to transforming the cells with the mRNA).

In particular examples the methods reduce infection in the subject with PID, such as reduce a sign or symptom of a bacterial, viral, fungal, or parasitic infection, such as one or more of a fever, swelling, redness, and pain. For example, the disclosed methods can reduce infection in a subject, such as reduce viral load, by at least 20%, at least 30%, at least 40%, at least 50%, at least 75%, at least 80%, at least 90%, at least 95%, at least 98%, at least 99%, or even 100%, (such a reduction of 20% to 50%, 40% to 50%, 20 to 75%, 40% to 60%, or 20% to 90%), for example as compared to no treatment with the disclosed methods (e.g., prior to treatment with the disclosed methods).

In some examples, combinations of these effects are achieved.

I. Obtaining Granulocytes, NK Cells, and Lymphocytes

Granulocytes, NK cells, and/or lymphocytes can be harvested from blood. In some examples, the granulocytes, NK cells, and/or lymphocytes are obtained from the same subject to be treated (autologous, i.e., the donor and recipient are the same person or subject).

The obtained autologous granulocytes, autologous NK cells, and/or autologous lymphocytes (which may be purified or isolated, or not, for example may be simply present in an apheresis product (e.g., leukapheresis product)), are transformed with mRNA encoding the defective or missing protein, thereby generating recombinant autologous granulocytes, recombinant autologous NK cells, and/or recombinant autologous lymphocytes. The population of granulocytes, NK cells, and/or lymphocytes used to generate the recombinant autologous granulocytes, recombinant autologous NK cells, and/or recombinant autologous lymphocytes, and the recombinant autologous granulocytes, recombinant autologous NK cells, and/or recombinant autologous lymphocytes administered to a subject, do not need to be 100% pure; lower amounts of purity are acceptable. For example, a population of cells that contains at least 50%, at least 60%, at least 70%, at least 80%, at least 90%, at least 95%, or at least 99% autologous granulocytes, autologous NK cells, and/or autologous lymphocytes, or recombinant autologous granulocytes and/or lymphocytes, can be used. In some examples, unpurified autologous granulocytes, autologous NK cells, and/or autologous lymphocytes are used, such as by directly using an apheresis (e.g., leukapheresis) product obtained, without isolating the autologous granulocytes, autologous NK cells, and/or autologous lymphocytes from the apheresis/leukapheresis product, prior to introducing the mRNA into such cells.

A. Peripheral Blood/Apheresis

To obtain granulocytes, NK cells, and/or lymphocytes from the circulating peripheral blood, subjects can be injected with a cytokine, such as granulocyte colony-stimulating factor (G-CSF) (e.g., filgrastim, Neupogen, Amgen), to induce cells to leave the bone marrow and circulate in the blood vessels. Side effects of G-CSF, including headache, bone pain, and myalgia, can be treated with acetaminophen or narcotics.

For example, the subject can be injected with G-CSF before the cell harvest. In one example, G-CSF (e.g., at least 5 mcg/kg/day, such as at least 10 mcg/kg/day, or at least 12 mcg/kg/day, such as about 5, 6, 7, 8, 9, 10, 11, 12, 13, 14, 15 or 16 mcg/kg/day) is administered subcutaneously to subjects daily for at least five days (such as 5, 6, 7, 8, 9 or 10 days). Doses can be given early in the morning, such as at least one hour and in some examples two hours prior to starting apheresis. G-CSF can be administered according to a vial-based algorithm to reduce wastage and increase the G-CSF dose given to lighter weight donors to improve yields as shown below:

| | |
|---|---|
| Donor Wt | Total G-CSF Dose (range) |

| | | | |
|---|---|---|---|
| 38-60 | kg | 600 mcg | (10.0 to 15.8 mcg/kg) |
| 61-78 | kg | 780 mcg | (10.0 to 12.8 mcg/kg) |
| 79-90 | kg | 900 mcg | (10.0 to 11.4 mcg/kg) |
| 91-96 | kg | 960 mcg | (10.0 to 10.5 mcg/kg) |
| 97-108 | kg | 1080 mcg | (10.0 to 11.0 mcg/kg) |
| >109 | kg | 1200 mcg | (<11.0 mcg/kg) |

A mobilized peripheral blood stem cell (PBSC) concentrate can then be collected by leukapheresis, for example about 2 hours after the last dose of G-CSF.

Donors can receive prophylactic continuous intravenous calcium chloride infusions to prevent citrate toxicity during apheresis. The volume processed per apheresis procedure can be determined by medical staff on the day of apheresis, based on peripheral blood white cell increase in response to G-CSF, optimum and minimum cell dose needed, and kilogram weight of recipient. Volume of blood processed can range from 12 to 30 liters per procedure for 1 to 2 consecutive daily procedures, not to exceed a total of 60 liters over 2 days. In pediatric subjects, defined as less than 40 kg, a maximum of 8 blood volumes can be processed per procedure, for up to 2 consecutive daily procedures. In children less than 18 kg undergoing autologous leukapheresis procedures, three additional considerations apply. A central venous double-lumen catheter can be used for apheresis. It may be necessary to “prime” the apheresis instrument with a unit of allogeneic red cells, due to the fact that the volume of blood in the device during apheresis will exceed the safe extracorporeal volume (SEV) allowed for the patient. The SEV is generally about 15% of circulating blood volume or 10.5 mL/kg. In extremely small children (less than 16 kg), it may not be possible to use citrate anticoagulant without risk of severe citrate toxicity, thus, systemic heparinization can be used during apheresis.

In some examples, subjects undergoing apheresis also receive calcium infusions (such as about 2 mg/mL).

In some examples, subjects undergoing apheresis also receive one dose of pleraxifor, for example if stem cells are also being collected.

The resulting apheresis sample can be used directly for the transformation of one or more mRNAs, or granulocytes and/or lymphocytes can be isolated from the apheresis product prior to transformation with the one or more mRNAs.

B. Isolation Methods

Once cells are obtained from the blood, the granulocytes, NK cells, and/or lymphocytes in the sample are optionally isolated or purified. However, in some examples the material obtained from the apheresis is used directly. Any methods of separating or isolating the granulocytes, NK cells, and/or lymphocytes from such samples can be used. Negative and positive selection methods can be used. Negative selection methods take advantage of cell surface markers which are not expressed on granulocytes, NK cells and/or lymphocytes. Positive selection methods take advantage of cell surface markers, such as CD34 and CD133 that are expressed on granulocytes, NK cells, and/or lymphocytes. In one example, hydroxyethyl starch (HES) is not used.

In one example, methods are used that deplete non-granulocytes and/or non-lymphocytes from the sample, thereby permitting enrichment of the granulocytes and/or lymphocytes (that is, negative selection). For example, methods that substantially reduce the number of B cells, T cells, NK cells, dendritic cells, monocytes, and/or red blood cells can be used. In one example, labeled antibodies specific for the undesired cells can be incubated with the sample, allowing the labeled antibodies to bind to the undesired cells. Separation methods can then be used to remove those cells from the sample. For example, if the antibody label (such as biotin) is mixed with ferromagnetic particles coated with streptavidin, then passing the mixture through columns in the presence of a magnetic field can be used to remove the undesired cells. Thus, after incubation with the labeled antibodies, the sample is applied to the column, such that undesired cells bind to the column, while the granulocytes and/or lymphocytes pass through the column and can be collected. In some examples, the label is a fluorophore and flow cytometry can be used to remove the cells. In some examples, methods are used to deplete RBCs, for example by incubating the apheresis product with ACK lysis buffer to lyse RBCs in the apheresis product.

Similarly, methods can be used to deplete non-NK cells, thereby enriching for NK cells.

In addition, commercially available kits can be used to deplete non-granulocytes, non-NK cells, and/or non-lymphocytes from the sample, such as those from Miltenyi.

In one example, methods are used that recover granulocytes, NK cells, and/or lymphocytes from the sample by elutriation, thereby permitting enrichment of the granulocytes and/or lymphocytes (positive selection). In one example, labeled antibodies specific for granulocytes, NK cells, and/or lymphocytes can be incubated with the sample, allowing the labeled antibodies to bind to the granulocytes, NK cells, and/or lymphocytes, and subsequent recovery of the labeled granulocytes, NK cells, and/or lymphocytes. In one example, the sample is exposed or incubated with labeled Miltenyi antibodies, thereby labeling the granulocytes, NK cells, and/or lymphocytes. The labeled granulocytes, NK cells, and/or lymphocytes can then be recovered, for example using flow cytometry (e.g., if the label is a fluorophore) or by use of a column (e.g., if the label is a magnetic label, such as magnetic beads containing CD3 for T cells).

The resulting granulocytes, NK cells, and/or lymphocytes can be used immediately to generate recombinant granulocytes, NK cells, and/or lymphocytes, or frozen for future use (for example frozen in growth media containing DMSO).

C. Culturing Granulocytes and/or Lymphocytes

The blood or apheresis product, or the isolated granulocytes, NK cells, and/or lymphocytes, can be cultured ex vivo, for example to expand the blood, apheresis product, granulocytes, NK cells, and/or lymphocytes, prior to introducing an exogenous mRNA into the cells. In some examples the cells (such as lymphocytes and/or NK cells) are grown in RPMI, plus animal or human serum, such as fetal calf serum. In some examples the growth media further includes cytokines, amino acids, and other growth supplements.

In some examples, the blood or apheresis product, or the isolated granulocytes, NK cells, and/or lymphocytes, are not cultured prior to transfection with the desired mRNA. For example, the resulting cells (e.g., granulocytes) can be washed in a buffer, such as an electroporation buffer containing albumin, such as 0.1 to 5% HSA, such as about 1% HSA.

II. Expressing mRNA to Correct Genetic Defect

Granulocytes, such as neutrophils, NK cells, and/or lymphocytes from the subject to be treated, are made recombinant by introducing one or more mRNAs encoding the defective or missing protein in the subject. The resulting recombinant granulocytes, recombinant NK cells, and/or recombinant lymphocytes, are introduced into the subject, wherein expression of the exogenous mRNA results in production of the defective or missing protein. Thus, the mRNA introduced into the granulocytes, NK cells, and/or lymphocytes is appropriate for the genetic defect in the subject, and expresses the defective/missing protein associated with the PID. In some examples, at least two different mRNAs are introduced into the autologous granulocytes and/or autologous lymphocytes, such as at least 3, at least 4, at least 5, at least 6, at least 7, at least 8, at least 9, or at least 10 different mRNAs. The different mRNAs can encode for different proteins, or can encode for the same protein (but have a different sequence), or both.

Although not necessary, detectable markers (e.g., fluorescent protein, such as GFP) or selection markers (e.g., antibiotic resistance) can be introduced along with the mRNA, to permit the identification of cells with the expressing the desired protein. In some examples introduced marker(s) is removed prior to introduction into the subject.

The autologous granulocytes, autologous NK cells, and/or autologous lymphocytes used can be previously cultured ex vivo (for example to increase their numbers), used directly following isolation from a blood or apheresis product, or used directly from a blood or an apheresis product (e.g., unpurified).

A. Manufacturing mRNAs

mRNA manufacturing processes that can be used include production of a plasmid DNA template or, in some cases, a DNA template that is generated using PCR, generation of a Master Cell Bank containing the plasmid DNA template (unless a PCR product is used as a DNA template), in vitro transcription (IVT), DNA template removal and RNA purification or clean-up, polyadenylation (or polyA tailing) and mRNA capping, and further purification processes as appropriate for the intended use. In some cases, such manufacturing steps are performed and documented using good manufacturing processes (GMP) that are compliant with guidances of the U.S. Food and Drug Administration or similar regulatory bodies in other countries or jurisdictions (e.g., the European Union).

1. Overview

The mRNAs used in the disclosed therapeutic and other methods can be of high quality and purity. For example, the mRNA can be at least 90% pure, at least 95% pure, at least 98% pure, at least 99% pure, at least 99.9% pure, or at least 99.99% pure. For example, in some cases, at least 90%, at least 95%, at least 98%, at least 99%, at least 99.9% or at least 99.99% of the stained material (e.g., with SYBR Gold) is in a single size band of an IVT-RNA, polyadenylated RNA, 5′-capped or polyadenylated IVT-RNA, or mRNA composition when said IVT-RNA, polyadenylated RNA, 5′-capped or polyadenylated IVT-RNA, or mRNA is separated based size on in a non-denaturing agarose electrophoresis gel, when said stained gel is visualize on a bioimager that detects the particular stain. In some examples, the mRNA is pure of contaminants that, if present, would induce an innate immune response if said mRNA does not induce a substantial interferon response, such as an alpha interferon response, in the subject receiving the recombinant autologous cells. In some examples, such mRNAs have a native mRNA sequence comprising unmodified A, G, U, C nucleosides. In other examples, such mRNAs include at least one modified nucleoside (e.g., pseudouridine) and, therefore has a non-native mRNA sequence. In one example, mRNAs include a cap at the 5′-end and a poly-A tail (such as ≥150 A's) at the 3′-end. In some other examples, mRNAs include a cap at the 5′-end, a poly-A tail, a 5′-UTR comprising a Kozak sequence, and 3′-UTR. In some examples, mRNAs (1) are codon optimized for the cell into which they are introduced, (2) include a human or Xenopus beta-globin 5′-UTR (5′ untranslated region) (which can include a Kozak sequence), (3) a human or Xenopus beta-globin 3′-UTR, (4) pseudouridines in place of all or substantially all (such as at least at least 95%, at least 98%, at least 99%) of the uridines in the ORF, (5) cap at the 5′-end, and (6) a poly-A tail (such as ≥150 A's) at the 3′end. If present, a 3′-UTR sequence immediately follows the translation termination codon and appears before the poly-A tail.

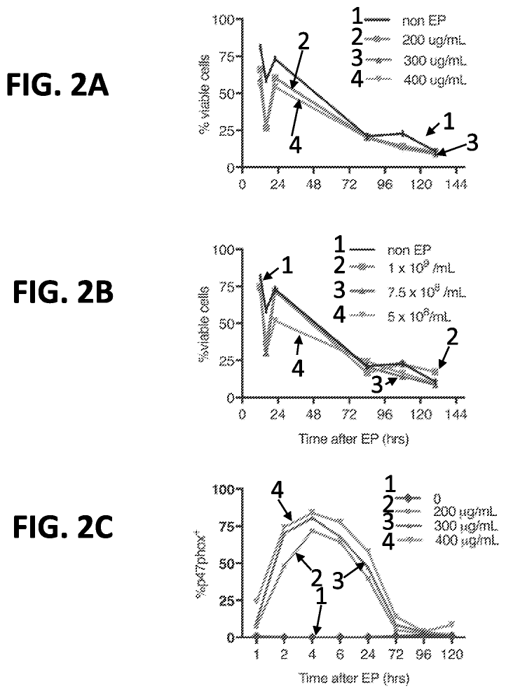

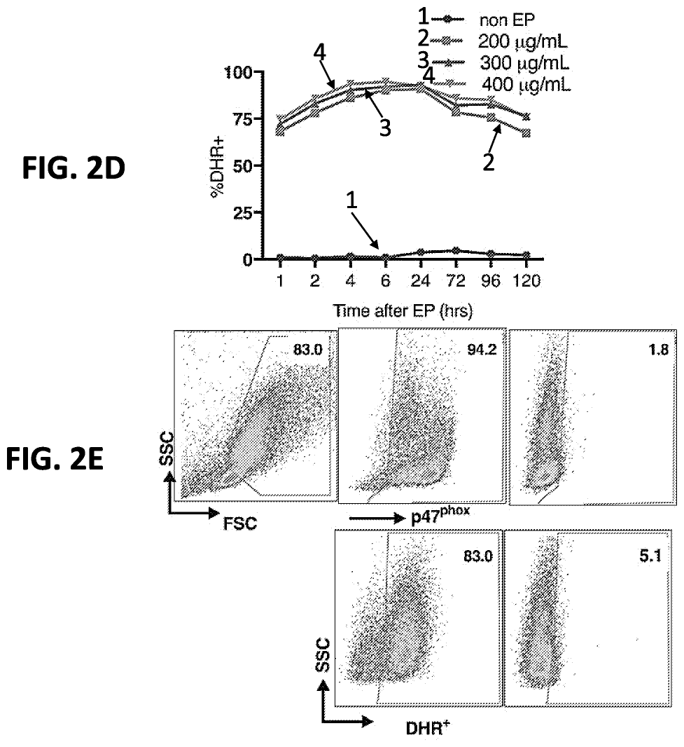

In some examples, the therapeutic mRNA introduced (e.g., via electroporation) into cells (e.g., granulocytes, lymphocytes or NK cells) is non-integrating and undergoes rapid degradation. In such transient gene therapy methods, any potential genotoxicity or extended action by the therapeutic mRNA can be reduced. Additional design features of the mRNA can include the use of modified nucleosides like pseudouridine (Kariko et al., Mol Ther 20:948-953, 2012). Even if mRNA is degraded rapidly, the resulting protein may survive longer conferring longer-term effects. In some examples, the protein-encoding exogenous mRNAs are designed and made for optimal protein expression and to avoid or reduce innate immune responses in the cells to be corrected. For example, as shown in FIG. 2D, NADPH oxidase activity following p47^{phox} mRNA correction was maintained out to 5 days. The therapeutic effect of such transient cells could be extended by repeated transfections and administration.

The mRNA can encode a native protein sequence, or a non-native protein sequence that includes silent mutations that result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. A codon optimized DNA or RNA sequence can have little sequence identity or sequence similarity with the natural or wild-type sequence because such a higher percentage of the codons may have changed. But the protein encoded remains unchanged.

In some examples, the mRNA used in the disclosed methods is codon optimized for the cell into which it is introduced. Codon preferences and codon usage tables for a particular species can be used to engineer mRNA molecules encoding a protein missing or defective in a subject with a PID that take advantage of the codon usage preferences of that particular species. For example, the mRNA expressed in the immune cells can be designed to have codons that are preferentially used by a particular organism of interest (e.g., in one whom the therapy is introduced). For example, if the subject is a human, the mRNA can be optimized for expression in a human cell, while if the subject is a mouse, the mRNA can be optimized for expression in a mouse cell.

The mRNA introduced into the granulocytes, NK cells, and/or lymphocytes can be a naked mRNA. Thus one or more mRNAs can be introduced into the autologous granulocytes, autologous NK cells, and/or autologous lymphocytes, to allow expression of the mRNA in the immune cells. In some examples, the mRNA introduced into the granulocytes, NK cells, and/or lymphocytes is part of a vector, such as a viral vector.

The disclosure provides isolated mRNA molecules encoding any protein deficient or defective in a subject with a PID, such as those listed in Table 1. For example, the mRNA can encode CD18/beta 2 integrin, MagT1, CTLA4, FoxP3, CD40Ligand, CARD9 (caspase recruitment domain-containing protein 9), or CARD11 (caspase recruitment domain-containing protein 11). Exemplary mRNAs are provided herein are shown in SEQ ID NOS: 1, 2, 4, 6, 8, 10, 12, 14, 16, 18, 20, 22, 24 and 26 which can be used to treat a PID or an infection or autoimmune disorder in such a subject. Thus the disclosure provides isolated mRNA molecules having at least 60%, at least 70%, at least 75%, at least 80%, at least 85%, at least 90%, at least 95%, at least 97%, at least 98%, at least 99%, or 100% sequence identity to any of SEQ ID NOS: 1, 2, 4, 6, 8, 10, 12, 14, 16, 18, 20, 22, 24 and 26 (wherein a T in such sequences can be a uridine in the mRNA sequence, or an unnatural nucleoside such as pseudouridine) as long as they encode a functional protein.

In some examples, the mRNA encodes a protein that is missing or defective in a CDG patient, such as encodes a native or wild-type CYBA (p22phox), CYBB (gp91phox), NCF1 (p47phox), NCF2 (p67phox), NCF4 (p40phox), or combinations thereof. In some examples, the mRNA encodes a protein having at least 80%, at least 85%, at least 90%, at least 95%, at least 97%, at least 98%, at least 99%, or 100% sequence identity to SEQ ID NO: 5, 7, 9, 11, 13, 15, 17, 21, 23, and 25. In some examples, the portion of the mRNA coding for the protein has at least 60%, at least 70%, at least 75%, at least 80%, at least 85%, at least 90%, at least 95%, at least 97%, at least 98%, at least 99%, or 100% sequence identity to SEQ ID NO: 1, 4, 6, 8, 10, 12, 14, 16, 20, 22, or 24 (wherein an mRNA can have a U or pseudouridine or other unnatural nucleoside in place of the Ts of SEQ ID NO: 1, 4, 6, 8, 10, or 12).

In some examples, the mRNA encodes a protein that is missing or defective in a XMEN patient, such as encodes a native or wild-type MagT1 protein. In some examples, the mRNA encodes a MagT1 protein having at least 80%, at least 85%, at least 90%, at least 95%, at least 97%, at least 98%, at least 99%, or 100% sequence identity to SEQ ID NO: 3 or 29. In some examples, the portion of the mRNA coding for the protein has at least 60%, at least 70%, at least 75%, at least 80%, at least 85%, at least 90%, at least 95%, at least 97%, at least 98%, at least 99%, or 100% sequence identity to SEQ ID NO: 2 or 18 (wherein an mRNA can have a U or pseudouridine or other unnatural nucleoside in place of the Ts of SEQ ID NO: 2).

In some examples, the mRNA encodes a protein that is missing or defective in a CTLA4 deficient patient, such as encodes a native or wild-type CTLA4 protein. In some examples, the mRNA encodes a CTLA4 protein having at least 80%, at least 85%, at least 90%, at least 95%, at least 97%, at least 98%, at least 99%, or 100% sequence identity to SEQ ID NO: 27. In some examples, the portion of the mRNA coding for the protein has at least 60%, at least 70%, at least 75%, at least 80%, at least 85%, at least 90%, at least 95%, at least 97%, at least 98%, at least 99%, or 100% sequence identity to SEQ ID NO: 26.

The sequences shown in SEQ ID NOS: 1, 2, 4, 6, 8, 10, 12, can have their Ts replaced with uridines, or an unnatural nucleoside such as pseudouridine, in place of the Ts. Thus, in some examples, the portion of the mRNA coding for the protein has at least 60%, at least 70%, at least 75%, at least 80%, at least 85%, at least 90%, at least 95%, at least 97%, at least 98%, at least 99%, or 100% sequence identity to SEQ ID NOS 1, 2, 4, 6, 8, 10, or 12, but has their Ts replaced with uridines, or an unnatural nucleoside such as pseudouridine.

In some examples, the portion of the mRNA coding for the protein has at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26. As noted above, a coding sequence shown in SEQ ID NOS 1, 2, 4, 6, 8, 10, or 12, can have their Ts replaced with uridines, or an unnatural nucleoside such as pseudouridine. mRNAs introduced in the disclosed autologous granulocytes, autologous NK cells, and/or autologous lymphocytes (generating recombinant cells) and used in the disclosed methods can include such mRNA coding sequences, and can further include (1) a 5′-end cap, (2) a 3′-end poly-A tail (such as 150 or more As), (3) a 3′-UTR (such as a human or Xenopus beta globin 3′-UTR, such as one having at least 60%, at least 70%, at least 75%, at least 80%, at least 85%, at least 90%, at least 95%, at least 97%, at least 98%, at least 99%, or 100% sequence identity to SEQ ID NO: 30 or 31); (4) a 5′-UTR (such as a human or Xenopus beta globin 5′-UTR, such as one including at least 60%, at least 70%, at least 75%, at least 80%, at least 85%, at least 90%, at least 95%, at least 97%, at least 98%, at least 99%, or 100% sequence identity to SEQ ID NO: 28 or 29) (which can include a Kozak sequence), (5) can be codon optimized for expression in a human cell, and/or (6) include one or more pseudouridines or other unnatural nucleoside in place of one or more uridines (or Ts) (e.g., replace at least 90%, at least 95%, at least 99%, or 100% of all U or Ts).

Thus, in one example, the mRNA includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26 (wherein the mRNA can have a U or pseudouridine or other unnatural nucleoside in place of the Ts of SEQ ID NO: 1, 2, 4, 6, 8, 10, or 12), and a 5′-end cap. In one example, the mRNA includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26 (wherein the mRNA can have a U or pseudouridine or other unnatural nucleoside in place of the Ts of SEQ ID NO: 1, 2, 4, 6, 8, 10, or 12), and a 3′-end poly-A tail (such as 150 or more As). In one example, the mRNA includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26 (wherein the mRNA can have a U or pseudouridine or other unnatural nucleoside in place of the Ts of SEQ ID NO: 1, 2, 4, 6, 8, 10, or 12), and a 3′-UTR (such as a human or Xenopus beta globin 3′-UTR, such as one having at least 60%, at least 70%, at least 75%, at least 80%, at least 85%, at least 90%, at least 95%, at least 97%, at least 98%, at least 99%, or 100% sequence identity to SEQ ID NO: 30 or 31). In one example, the mRNA includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26 (wherein the mRNA can have a U or pseudouridine or other unnatural nucleoside in place of the Ts of SEQ ID NO: 1, 2, 4, 6, 8, 10, or 12), and a 5′-UTR (such as a human or Xenopus beta globin 5′-UTR, such as one having at least 60%, at least 70%, at least 75%, at least 80%, at least 85%, at least 90%, at least 95%, at least 97%, at least 98%, at least 99%, or 100% sequence identity to SEQ ID NO: 28 or 29) (which can include a Kozak sequence). In one example, the mRNA includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26 (wherein the mRNA can have a U or pseudouridine or other unnatural nucleoside in place of the Ts of SEQ ID NO: 1, 2, 4, 6, 8, 10, or 12), wherein the coding sequence is codon optimized for expression in a human cell. In one example, the mRNA includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26 (wherein the mRNA can have a U or pseudouridine or other unnatural nucleoside in place of the Ts of SEQ ID NO: 1, 2, 4, 6, 8, 10, or 12), wherein one or more uridines (or Ts) are replaced with pseudouridine or other unnatural nucleoside (e.g., least 90%, at least 95%, at least 99%, or 100% of all U or Ts replaced). Combinations of such are also envisioned.

In one example, the mRNA includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26 (wherein the mRNA can have a U or pseudouridine or other unnatural nucleoside in place of the Ts of SEQ ID NO: 1, 2, 4, 6, 8, 10, or 12), and a 5′-end cap and a 3′-end poly-A tail (such as 150 or more As). In one example, the mRNA includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26 (wherein the mRNA can have a U or pseudouridine or other unnatural nucleoside in place of the Ts of SEQ ID NO: 1, 2, 4, 6, 8, 10, or 12), and a 3′-UTR (such as a human or Xenopus beta globin 3′-UTR, such as one having at least 60%, at least 70%, at least 75%, at least 80%, at least 85%, at least 90%, at least 95%, at least 97%, at least 98%, at least 99%, or 100% sequence identity to SEQ ID NO: 30 or 31) and a 5′-UTR (such as a human or Xenopus beta globin 5′-UTR, such as one having at least 60%, at least 70%, at least 75%, at least 80%, at least 85%, at least 90%, at least 95%, at least 97%, at least 98%, at least 99%, or 100% sequence identity to SEQ ID NO: 28 or 29) (which can include a Kozak sequence). In one example, the mRNA includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26, wherein the coding sequence is codon optimized for expression in a human cell and wherein one or more uridines (or Ts) are replaced with pseudouridine or other unnatural nucleoside (e.g., least 90%, at least 95%, at least 99%, or 100% of all U or Ts replaced). Combinations of such are also envisioned.

In one example, the mRNA includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26 (wherein the mRNA can have a U or pseudouridine or other unnatural nucleoside in place of the Ts of SEQ ID NO: 1, 2, 4, 6, 8, 10, or 12), and a 5′-end cap, a 3′-end poly-A tail (such as 150 or more As), a 3′-UTR (such as a human or Xenopus beta globin 3′-UTR, such as one having at least 60%, at least 70%, at least 75%, at least 80%, at least 85%, at least 90%, at least 95%, at least 97%, at least 98%, at least 99%, or 100% sequence identity to SEQ ID NO: 30 or 31) and a 5′-UTR (such as a human or Xenopus beta globin 5′-UTR, such as one having at least 60%, at least 70%, at least 75%, at least 80%, at least 85%, at least 90%, at least 95%, at least 97%, at least 98%, at least 99%, or 100% sequence identity to SEQ ID NO: 28 or 29) (which can include a Kozak sequence). In one example, such an mRNA has a coding sequence is codon optimized for expression in a human cell. In one example, such an mRNA has one or more uridines (or Ts) replaced with pseudouridine or other unnatural nucleoside (e.g., least 90%, at least 95%, at least 99%, or 100% of all U or Ts replaced). Combinations of such are also envisioned.

In one example, the mRNA includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26 (wherein the mRNA can have a U or pseudouridine or other unnatural nucleoside in place of the Ts of SEQ ID NO: 1, 2, 4, 6, 8, 10, or 12), a 5′-end cap, a 3′-end poly-A tail (such as 150 or more As), a 3′-UTR (such as a human or Xenopus beta globin 3′-UTR, such as one having at least 60%, at least 70%, at least 75%, at least 80%, at least 85%, at least 90%, at least 95%, at least 97%, at least 98%, at least 99%, or 100% sequence identity to SEQ ID NO: 30 or 31), a 5′-UTR (such as a human or Xenopus beta globin 5′-UTR, such as one having at least 60%, at least 70%, at least 75%, at least 80%, at least 85%, at least 90%, at least 95%, at least 97%, at least 98%, at least 99%, or 100% sequence identity to SEQ ID NO: 28 or 29) (which can include a Kozak sequence), has a coding sequence is codon optimized for expression in a human cell, and has one or more uridines (or Ts) replaced with pseudouridine or other unnatural nucleoside (e.g., least 90%, at least 95%, at least 99%, or 100% of all U or Ts replaced).

2. DNA Template Manufacturing

mRNAs can be synthesized by in vitro transcription of a DNA template comprising a coding region for a native or wild-type or functional form of a protein that is deficient or absent in a PID patient. The DNA template can be designed to include the coding region and additional sequences, such as a 5′ untranslated region (5′ UTR) (such as one containing a Kozak sequence) and a 3′-untranslated region (3′ UTR) for the desired mRNA encoding the protein. For example, such DNA templates can be made by RT-PCR of a desired mRNA in total cellular RNA extracted from a cell that contains said desired mRNA using as PCR primers, or by assembly of commercially obtained oligodeoxyribonucleotides made based on the known GenBank or other databases of human DNA and mRNA coding region sequences for said protein. During template production, the natural or wild-type sequence of a coding sequence of interest can be modified by codon optimization to improve protein expression of the mRNA. Thus, in some cases, the sequence of the DNA template is codon optimized for human gene expression using an online tool provided by an oligodeoxyribonucleotides manufacturer (e.g., Integrated DNA Technologies, Inc., Skokie, Ill., USA). This codon optimization tool converts the DNA sequence of any chosen GenBank sequence encoding a protein of interest into a new DNA sequence that is optimized for protein expression. In part, this is accomplished because the tool helps the user pick from among the 61 codons that encode the 20 standard amino acids which ones should be optimal codon use for gene expression in a human cell. Many companies offer similar codon optimization tools. Once the codon optimized DNA is designed, oligodeoxyribonucelotides can be prepared for assembling the DNA template. The codon optimized DNA sequence may have low sequence identity or sequence similarity to the natural or wild-type coding sequence because up to one-third or more of the codons could be changed; nevertheless, codon optimization alone (i.e., without making sequence changes for other reasons) does not change the amino acid sequence, even if the DNA template and mRNA sequences have changed significantly.

DNA templates obtained using these methods may be ligated into a linearized plasmid, e.g., one that contains a promoter for RNA polymerase which is to be used for in vitro transcription of the DNA template, thereby operably joining the promoter sequence to the coding region of the gene encoding the desired protein. The resulting plasmid comprising the coding region can be used to transform an E. coli strain (or other microbial or eukaryotic cell) to make a Master Cell Bank containing the plasmid, which can then be qualified for use in GMP manufacturing of in vitro-transcribed RNA encoding the protein of interest that is deficient in PID patients for use in therapy according to the methods described herein. Plasmid purified from the Master Cell Bank is sequenced prior to it being used for RNA production to confirm that the DNA sequence of the coding region encoding the desired protein of interest is accurate and that it can be used to compensate for the protein that is missing or deficient in PID patients. The Master Cell Bank can be used to generate template for each subsequent GMP manufacturing of plasmid template. Prior to it being used for making RNA by in vitro transcription, the closed circular plasmid DNA template is linearized at a restriction enzyme site downstream of the DNA coding region.

3. mRNA Production

The linearized plasmid DNA template produced as described above can be used to manufacture mRNA by in vitro transcription (IVT) using an RNA polymerase (RNAP). Exemplary RNAPs that can be used include, but are not limited to, T7 RNAP, T3 RNAP and mutant polymerases such as, but not limited to, polymerases able to incorporate modified nucleic acids. The RNAP initiates transcription at a cognate RNAP promoter (e.g., a T7 promoter for T7 RNAP, a T3 promoter for T3 RNAP or an SP6 promoter for SP6 RNAP. The in vitro transcription system typically includes a transcription buffer, ribonucleoside-5′-triphosphates (NTPs), an RNase inhibitor, dithiothreitol (DTT) and the RNAP. The NTPs may be selected from, but are not limited to, those described herein including the natural or canonical NTPs consisting of ATP, CTP, GTP and UTP, as well as unnatural or modified NTPs, such as the ribonucleoside-5′-triphosphates of modified nucleosides (e.g., pseudouridine (φ), 1-methylpseudouridine (m¹φ), 5-methyluridine (m⁵U), 5-methoxyuridine (mo⁵U) and 2-thiouridine (s²U) and 5-methylcytidine (m⁵C)). T7 RNAP incorporates the ribonucleoside-5′-triphosphates of φ, m¹φ, m⁵U, mo⁵U, s²U and m⁵C into IVT-RNA. One or more of the NTPs (e.g., pseudouridine-5′-triphosphate or φTP) may be manufactured in house (e.g., using FDA-compliant GMP processes) or may be obtained from a commercial supplier.

To perform IVT, the following solutions can be combined: RNase-free water, linearized DNA template DNA, enough concentrated RNAP transcription buffer to generate the final desired concentration, enough concentrated nucleoside-5′-triphosphate solution (e.g., NTP solution containing concentrated equimolar concentrations of ATP, GTP, CTP and either UTP or φTP (pseudouridine-5′-triphosphate) to generate the final desired concentration, concentrated dithiothreitol to generate the final desired concentration, an RNase inhibitor (if desired) and a concentrated solution of the respective RNA polymerase to generate an appropriate enzymatic activity per the suppliers specifications. If it is desired to synthesize capped RNA by incorporation of a dinucleotide cap analog (e.g., an anti-reverse cap analog or ARCA) during the IVT reaction, then said dinucleotide cap analog is substituted for a portion of the GTP in the IVT reaction. The RNAP will then incorporate the ARCA cap at the 5′ end of the message in approximately the same proportion as its molar concentration relative to the molar concentration of GTP in the IVT reaction; e.g., if the relative molar concentrations of ARCA to GTP is 4/1, then about 80% of the RNA will be capped and 20% of the IVT-RNA will have a 5′-triphosphate group. If high concentrations of NTPs are used, the IVT reaction is generally incubated for a period of time as recommended by the supplier of the IVT RNA polymerase enzyme or kit (e.g., at about 37° C. for 30 to 60 minutes).

4. Purification of the In Vitro—Transcribed RNA (IVT-RNA)

The DNA template used for IVT may be digested using recombinant animal-origin free deoxyribonuclease preparation. Then, the IVT-RNA can be purified by organic extraction followed by ammonium acetate precipitation, which removes proteins and selectively precipitates RNA, leaving most of the DNA and unincorporated NTPs in the supernatant. Organic extractions, such as with 70% ethanol, may also be employed.

5. Polyadenylation of the IVT-RNA

The purified IVT-RNA is then polyadenylated enzymatically, for example using A-Plus™ PolyA Polymerase as described by the manufacturer (CELLSCRIPT, Madison, Wis., USA) or another commercially available polyA polymerase as described by said manufacturer. Polyadenylated IVT-RNA may be further purified as described for the IVT-RNA.

6. Capping of Polyadenylated IVT-RNA

The polyadenylated IVT-RNA is enzymatically capped, for example using ScriptCap™ Capping Enzyme, to generate 5′-capped and 3′-polyadenylated IVT-RNA having a cap with a cap0 structure, or, if desired, the polyadenylated IVT-RNA is enzymatically capped using both ScriptCap™ Capping Enzyme and ScriptCap™ 2′-O-Methyltransferase to generate 5′-capped and 3′-polyadenylated IVT-RNA having a cap with a cap1 structure as described by the manufacturer (CELLSCRIPT, Madison, Wis., USA). 5′-Capped and polyadenylated IVT-RNA may be further purified as described for the IVT-RNA.

Additional details of the above materials and processes and other information, such as chemical structures for 5′ caps with cap0 and cap1 structures are available in the online pdf files on the Products page at www.cellscript.com.

7. Other Purification Processes for IVT-RNA, Polyadenylated RNA, 5′-Capped and Polyadenylated IVT-RNA, or mRNA

In addition to the above processes, RNA (including IVT-RNA, polyadenylated RNA, 5′-capped and polyadenylated IVT-RNA or mRNA) for therapeutic or other uses in or on humans or animals may be further purified using other purification processes, such as using processes that include HPLC or gravity flow column purification of mRNA and/or treatment of said RNA with RNase III, followed by clean-up steps to remove the enzyme and other enzyme digestion products (e.g., as disclosed in U.S. Pat. No. 10,201,620).

In some examples, RNA for therapeutic or other uses involving administration to humans or animals or cells therefrom, are purified such that said RNA is free of double-stranded RNA (dsRNA) having a length of greater than about 40 basepairs (bps) in length. For example, in some cases, the amount of dsRNA in said purified RNA for therapeutic or other use in humans or animals (for example present in a therapeutic composition) is quantified using an immunofluorescent assay to detect the amount of a dsRNA-specific mAb that binds to dsRNA that is present in RNA spotted on nylon membrane dot blots on which controls containing known amounts of the tested RNA or dsRNA are also spotted as controls. For example, in some cases using this assay, the amount of dsRNA contaminant molecules that have a size of greater than 40 basepairs (bp) in length are quantified using a dsRNA-specific J2 mAb or K1 mAb (e.g., from English & Scientific Consulting, Kft., Szirik, Hungary (also known as SCICONS)), which mAbs have the IgG isotype (igG2a subclass) and only efficiently bind dsRNA longer than 40 bp in length, in a sequence-independent manner.

In some cases, the amount of dsRNA contaminant molecules having a size of greater than 40 bp in said RNA following purification of RNA for therapeutic or other use in humans or animals following purification is less than 0.1% of the total mass (i.e., weight) of the RNA. In some cases, the amount of dsRNA contaminant molecules having a size of greater than 40 bp in said RNA following purification of the RNA is less than 0.01% of the total mass (i.e., weight) of the RNA. In some cases, the amount of dsRNA RNA contaminant molecules that have a size of greater than 40 bp in said RNA following purification is less than 0.001% of the total mass (i.e., weight) of the RNA.

For example, in some cases, said RNA (e.g., 25-100 ng) can be blotted onto a nitrocellulose membrane, allowed to dry, blocked with 5% non-fat dried milk in TBS buffer supplemented with 0.05% Tween-20 (TBS-T), and incubated with a dsRNA-specific J2 mAb or K1 mAb for 60 minutes. Membranes are washed six times with TBS-T and then reacted with HRP-conjugated donkey anti-mouse antibody (Jackson Immunology). After washing six times, dsRNA can be detected with the addition of SuperSignal West Pico Chemiluminescent substrate (Pierce) and image capture for 30 seconds to two minutes on a Fujifilm LAS1000 digital imaging system. In still other immunofluorescent assays of the amount of dsRNA in purified RNA using the J2 or K1 mAbs can be carried out using a second Ab that has different label and the amount of dsRNA in said RNA is quantified using a digital bioimager that can detect said different label.

8. RNA Compositions for Treating a PID

In some examples, the IVT-RNA, polyadenylated RNA, 5′-capped RNA, 5′-capped and polyadenylated IVT-RNA, or mRNA composition, kit, or method of use has specifications, comprises, is for use in, or is for use in making a pharmaceutical product or a pharmaceutical composition for treating a PID or is for a therapeutic use for treating a PID. Thus in some examples, the therapeutic mRNA used in the disclosed methods, or present in a disclosed cell or composition, includes one of the following:
a. One exemplary composition is an IVT-RNA, polyadenylated RNA, 5′-capped and polyadenylated IVT-RNA, or mRNA composition that encodes a native or wild-type or functional protein that compensates for a protein that is deficient or absent in a PID patient, wherein all or substantially all (such as at least 95%, at least 98%, or at least 99%) of the nucleosides in the open reading frame comprises guanosine, adenosine, cytidine and uridine.
b. Another exemplary composition is an IVT-RNA, polyadenylated RNA, 5′-capped and polyadenylated IVT-RNA, or mRNA composition that encodes a native or wild-type or functional protein that compensates for a protein that is deficient or absent in a PID patient, wherein pseudouridine residues are present in place of all or substantially all (such as at least 95%, at least 98%, or at least 99%) of the uridine nucleosides.
c. Another exemplary composition is an IVT-RNA, polyadenylated RNA, 5′-capped and polyadenylated IVT-RNA, or mRNA composition that encodes a native or wild-type or functional protein that compensates for a protein that is deficient or absent in a PID patient, wherein 1-methylpseudouridine residues are present in place of all or substantially all (such as at least 95%, at least 98%, or at least 99%) of the uridine nucleosides.
d. Another exemplary composition is an IVT-RNA, polyadenylated RNA, 5′-capped and polyadenylated IVT-RNA, or mRNA composition that encodes a native or wild-type or functional protein that compensates for a protein that is deficient or absent in a PID patient, wherein 5-methyluridine (m5U) residues are present in place of all or substantially all (such as at least 95%, at least 98%, or at least 99%) of the uridine nucleosides.
e. Another exemplary composition is an IVT-RNA, polyadenylated RNA, 5′-capped and polyadenylated IVT-RNA, or mRNA composition that encodes a native or wild-type or functional protein that compensates for a protein that is deficient or absent in a PID patient, wherein, 5-methoxyuridine (mo5U) residues are present in place of all or substantially all (such as at least 95%, at least 98%, or at least 99%) of the uridine nucleosides.
f. Another exemplary composition is an IVT-RNA, polyadenylated RNA, 5′-capped and polyadenylated IVT-RNA, or mRNA composition that encodes a native or wild-type or functional protein that compensates for a protein that is deficient or absent in a PID patient, wherein 2-thiouridine (s2U) residues are present in place of between about 10% and about 60% of the uridine nucleosides.
g. Another exemplary composition is an IVT-RNA, polyadenylated RNA, 5′-capped and polyadenylated IVT-RNA, or mRNA compositions of any of A, B, C, D, E or F, 5-methylcytidine (m5C) residues are present in place of all or substantially all (such as at least 95%, at least 98%, or at least 99%) of the cytidine nucleosides.
h. Another exemplary composition is an IVT-RNA, polyadenylated RNA, 5′-capped and polyadenylated IVT-RNA, or mRNA compositions any of A, B, C, D, E, F or G, said IVT-RNA, polyadenylated RNA, 5′-capped and polyadenylated IVT-RNA, or mRNA composition comprises at least one β-globin 5′ UTR or at least one β-globin 3′ UTR.
i. Another exemplary composition is an IVT-RNA, polyadenylated RNA, 5′-capped and polyadenylated IVT-RNA, or mRNA compositions of H, the at least one 3-globin 5′ UTR or at least one β-globin 3′ UTR is from a human or a Xenopus β-globin gene.
j. Another exemplary composition is an IVT-RNA, polyadenylated RNA, 5′-capped and polyadenylated IVT-RNA, or mRNA composition any of a. through i, wherein said IVT-RNA, polyadenylated RNA, 5′-capped and polyadenylated IVT-RNA, or mRNA composition comprises or has a 5′ cap with a cap0 structure comprising a m7guanosine (m7G) cap nucleotide.
k. Another exemplary composition is an IVT-RNA, polyadenylated RNA, 5′-capped and polyadenylated IVT-RNA, or mRNA composition of j., said IVT-RNA, polyadenylated RNA, 5′-capped and polyadenylated IVT-RNA, or mRNA composition comprising or having a 5′ cap with a cap1 structure, wherein in addition to the m7G cap nucleotide, the penultimate nucleotide to the 5′ cap nucleotide comprises a 2′-O-methyl group.
l. The IVT-RNA, polyadenylated RNA, 5′-capped and polyadenylated IVT-RNA, or mRNA compositions of any of a through k above, wherein said IVT-RNA, polyadenylated RNA, 5′-capped and polyadenylated IVT-RNA has been purified using a process comprising HPLC or gravity flow chromatography, such that said IVT-RNA, polyadenylated RNA, 5′-capped and polyadenylated IVT-RNA, said IVT-RNA, polyadenylated RNA, 5′-capped and polyadenylated IVT-RNA, or mRNA composition is free of RNA contaminants that are immunogenic by inducing an innate immune response, as is detectable using an in vitro MDDC immunogenicity assay, wherein the amount of TNF-α secreted by monocyte-derived dendritic cells (MDDCs) transfected with said IVT-RNA, polyadenylated RNA, 5′-capped and polyadenylated IVT-RNA, or mRNA composition is not greater than the amount of TNF-α secreted by mock transfected MDDC negative controls that lack any RNA. In some examples, innate immunogenicity of an IVT-RNA, polyadenylated RNA, 5′-capped and polyadenylated IVT-RNA, or mRNA composition is further detectable by measuring secretion of one or more cytokines, such as IL-12, IFN-α, TNF-α, RANTES, MIP-1α, MIP-1, IL-6, IFN-Rβ or IL-8, from murine dendritic cells or from human monocyte-derived dendritic cells or from other cells with toll-like receptors (TLRs, e.g., TLR3, TLR7 or TLR8) which can detect and initiate signaling pathways that result in the cell secreting one or more specific cytokines in response to the presence of such innate immunogenic contaminants.
Further, the processes for purification of an IVT-RNA, polyadenylated RNA, 5′-capped and polyadenylated IVT-RNA, or mRNA composition can be monitored using one or more of these assays to detect the presence and relative levels of contaminants, including RNA contaminants, that are immunogenic (and toxic to cells) by inducing an innate immune response that results in increased secretion of one or more cytokines (e.g., an inflammatory or pro-inflammatory cytokine). Assays for detecting and quantifying secretion of one or more cytokines (e.g., TNF-α from murine dendritic cells or from human monocyte-derived dendritic cells or other cells with toll-like receptors (TLRs, e.g., TLR3, TLR7 or TLR8), which can detect and signal the cell to secrete one or more specific cytokines in response to the presence of such innate immunogenic contaminants), can be used. In some examples, such assays for secretion of one or more cytokines can be used to measure fractions from gravity flow or high- or medium- or low-pressure chromatographic columns or fraction or samples from any other purification processes that are is used to purify such IVT-RNA, polyadenylated RNA, 5′-capped and polyadenylated IVT-RNA, or mRNA composition. Such cytokine assays permit detection and relative quantification of the levels of immunogenic contaminants, including immunogenic RNA contaminants, enabling purification of such desired RNA molecules to much higher levels of purity.
Methods can be used to generate human or murine monocyte-derived dendritic cells (MDDCs), which can be used for such cytokine assays. For example, monocytopheresis is used to obtain monocyte samples from normal human volunteers and then monocytes are treated with GM-CSF (50 ng/ml)+IL-4 (100 ng/ml) (R&D Systems) in AIM V medium (Invitrogen) for 7 days. On days 3 and 6, a 50% volume of new medium with cytokines is added. Alternatively, human MDDCs can be obtained from commercial sources. Murine DCs can be generated by isolating bone marrow mononuclear cells from Balb/c mice and culturing in RPMI+10% FBS medium supplemented with murine GM-CSF (20 ng/ml, Peprotech). On days 3 and 6, a 50% volume of new medium with GM-CSF was added. Non-adherent cells can be used after 7 days of culture.
Many methods and ready-to-use kits are available for using MDDCs to assay for the presence of substances that result in secretion of one or more cytokines. For example, in one assay, MDDCs in 96-well plates (containing approximately 100,000 cells/well) are treated with R-848, Lipofectin® alone, or Lipofectin®-complexed RNA for 1 hour. Then, then the medium is changed, and the cells incubated for a defined period of time (e.g., overnight or 8-24 hours), after which, cells are harvested for RNA isolation or flow cytometry, and the collected culture medium is subjected to ELISA (e.g., to measure levels of TNF-α and/or IFN-α and/or other cytokines in supernatants by sandwich ELISA. For example, each culture condition can be performed in triplicate or quadruplicate and measured in duplicate.
m. The IVT-RNA, polyadenylated RNA, 5′-capped and polyadenylated IVT-RNA, or mRNA compositions of any of a. through., wherein said IVT-RNA, polyadenylated RNA, 5′-capped and polyadenylated IVT-RNA has been purified using a process that includes treatment of said RNA with RNase III, followed by clean-up steps to remove the enzyme and other enzyme digestion products as disclosed in U.S. Pat. No. 10,201,620, such that said IVT-RNA, polyadenylated RNA, 5′-capped and polyadenylated IVT-RNA, said IVT-RNA, polyadenylated RNA, 5′-capped and polyadenylated IVT-RNA, or mRNA composition is free of RNA contaminants that are immunogenic by inducing an innate immune response, as is detectable using an in vitro MDDC immunogenicity assay, wherein the amount of TNF-α secreted by monocyte-derived dendritic cells (MDDCs) transfected with said IVT-RNA, polyadenylated RNA, 5′-capped and polyadenylated IVT-RNA, or mRNA composition is not greater than the amount of TNF-α secreted by mock transfected MDDC negative controls that lack any RNA.
n. The IVT-RNA, polyadenylated RNA, 5′-capped and polyadenylated IVT-RNA, or mRNA composition of any of a. through k., wherein said protein that is deficient or absent in a PID patient is phagocytic NADPH oxidase and said IVT-RNA, polyadenylated RNA, 5′-capped and polyadenylated IVT-RNA, or mRNA composition encodes a wild-type or functional protein comprising one or more NADPH oxidase a subunits, such as one or more of a gp91^{phox} subunit, a p47^{phox} subunit, p67^{phox} subunit, p22^{phox} subunit and p4^{phox} subunit.
o. The IVT-RNA, polyadenylated RNA, 5′-capped and polyadenylated IVT-RNA, or mRNA composition of n., for use in making a pharmaceutical composition for treating CGD or for use in a method for treating CGD.
p. The IVT-RNA, polyadenylated RNA, 5′-capped and polyadenylated IVT-RNA, or mRNA compositions of any of a. through k., wherein said protein that is deficient or absent in a PID patient is CD18 protein and said IVT-RNA, polyadenylated RNA, 5′-capped and polyadenylated IVT-RNA, or mRNA composition encodes a wild-type or functional CD18 protein.
q. The IVT-RNA, polyadenylated RNA, 5′-capped and polyadenylated IVT-RNA, or mRNA composition of p. for use in making a pharmaceutical composition for treating Leukocyte Adhesion Disease (LAD) or for use in a method for treating LAD.
r. The IVT-RNA, polyadenylated RNA, 5′-capped and polyadenylated IVT-RNA, or mRNA compositions of any of a. through k., wherein said protein that is deficient or absent in a PID patient is MagT1 protein and said IVT-RNA, polyadenylated RNA, 5′-capped and polyadenylated IVT-RNA, or mRNA composition encodes a wild-type or functional MagT1 protein.
s. The IVT-RNA, polyadenylated RNA, 5′-capped and polyadenylated IVT-RNA, or mRNA composition of r. for use in making a pharmaceutical composition for treating X-linked magnesium defect, Epstein-Barr virus infection and neoplasia (XMEN Disease) or for use in a method for treating XMEN Disease.
t. The IVT-RNA, polyadenylated RNA, 5′-capped and polyadenylated IVT-RNA, or mRNA compositions of any of a. through k., wherein said protein that is deficient or absent in a PID patient is CTLA4 protein and said IVT-RNA, polyadenylated RNA, 5′-capped and polyadenylated IVT-RNA, or mRNA composition encodes a wild-type or functional CTLA4 protein.
u. The IVT-RNA, polyadenylated RNA, 5′-capped and polyadenylated IVT-RNA, or mRNA composition of t. for use in making a pharmaceutical composition for treating CTLA4 deficiency or for use in a method for treating CTLA4 deficiency.
v. The IVT-RNA, polyadenylated RNA, 5′-capped and polyadenylated IVT-RNA, or mRNA compositions of any of a. through k., wherein said protein that is deficient or absent in a PID patient is CYBA (also known as p22phox) protein and said IVT-RNA, polyadenylated RNA, 5′-capped and polyadenylated IVT-RNA, or mRNA composition encodes a wild-type or functional CYBA protein.
w. The IVT-RNA, polyadenylated RNA, 5′-capped and polyadenylated IVT-RNA, or mRNA composition of v. for use in making a pharmaceutical composition for treating CYBA deficiency (also known as p22phox protein deficiency) or for use in a method for treating CYBA deficiency.
x. The IVT-RNA, polyadenylated RNA, 5′-capped and polyadenylated IVT-RNA, or mRNA compositions of any of a. through k., wherein said protein that is deficient or absent in a PID patient is CD40L protein and said IVT-RNA, polyadenylated RNA, 5′-capped and polyadenylated IVT-RNA, or mRNA composition encodes a wild-type or functional CD40L protein.
y. The IVT-RNA, polyadenylated RNA, 5′-capped and polyadenylated IVT-RNA, or mRNA composition of x. for use in making a pharmaceutical composition for treating CD40L deficiency or for use in a method for treating CD40L deficiency.
z. The IVT-RNA, polyadenylated RNA, 5′-capped and polyadenylated IVT-RNA, or mRNA compositions of any of a. through k., wherein said protein that is deficient or absent in a PID patient is DOCK8 protein and said IVT-RNA, polyadenylated RNA, 5′-capped and polyadenylated IVT-RNA, or mRNA composition encodes a wild-type or functional DOCK8 protein.
aa. The IVT-RNA, polyadenylated RNA, 5′-capped and polyadenylated IVT-RNA, or mRNA composition of z. for use in making a pharmaceutical composition for treating DOCK8 deficiency or for use in a method for treating DOCK8 deficiency.
bb. The IVT-RNA, polyadenylated RNA, 5′-capped and polyadenylated IVT-RNA, or mRNA compositions of any of a. through k., wherein said protein that is deficient or absent in a PID patient is IL12 protein and said IVT-RNA, polyadenylated RNA, 5′-capped and polyadenylated IVT-RNA, or mRNA composition encodes a wild-type or functional IL12 protein.
cc. The IVT-RNA, polyadenylated RNA, 5′-capped and polyadenylated IVT-RNA, or mRNA composition of bb. for use in making a pharmaceutical composition for treating IL12 deficiency or for use in a method for treating IL12 deficiency.
dd. The IVT-RNA, polyadenylated RNA, 5′-capped and polyadenylated IVT-RNA, or mRNA compositions of any of a. through k., wherein said protein that is deficient or absent in a PID patient is IL23 protein and said IVT-RNA, polyadenylated RNA, 5′-capped and polyadenylated IVT-RNA, or mRNA composition encodes a wild-type or functional IL23 protein.
ee. The IVT-RNA, polyadenylated RNA, 5′-capped and polyadenylated IVT-RNA, or mRNA composition of dd. for use in making a pharmaceutical composition for treating IL23 deficiency or for use in a method for treating IL23 deficiency.

9. 5′-Caps

In some examples, the mRNA used in the disclosed methods includes a cap on its 5′-end. The cap on the 5′-end of the mRNA can include a guanine nucleoside that is joined via its 5′-carbon to a triphosphate group that is, in turn, joined to the 5′-carbon of the most 5′-nucleotide of the IVT-RNA.

In some examples, the 5′-end of the mRNA is enzymatically capped with a capping enzyme with RNA triphosphatase, RNA guanyltransferase and guanine-7-methyltransferase activities, which yields an N⁷-methylguanosine standard cap with a cap0 structure. In some other cases, the mRNA with an N⁷-methylguanosine standard cap is further modified using 2′-O-methyltransferase, which 2′-O-methylates the 2′ position of the 5′-penultimate nucleotide with respect to the N⁷-methylguanosine standard cap, thereby generating a dinucleotide cap on the RNA which has a cap1 structure.

An anti-reverse cap analog (ARCA), which has a structure of m₂^{7,3-O} G(5′)ppp(5′)G; P-(5′-(3′-O-methyl)-7-methyl-guanosyl) P³-(5′-(guanosyl))triphosphate), can be used for transcription during the IVT. However, about 20% of the IVT-RNA may not be capped and will not be translated because the cap analog is usually present in a 4/1 molar ratio GTP during the IVT reaction.

In one example, the cap on the 5′-end of the mRNA is

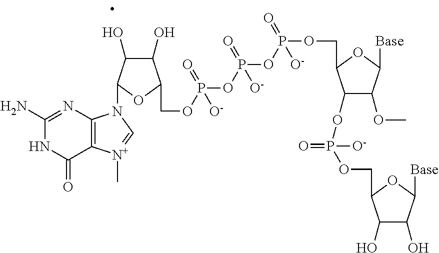

Other exemplary 5′-end caps (and methods on how to incorporate them onto a nucleic acid molecule) are provided in US Application Publication No. 20140221248 (herein incorporated by reference).

10. 3′-Poly-A Tails

In some examples, the mRNA used in the disclosed methods includes a poly-A tail at its 3′-end. In some examples, the poly-A tail is at least 150 adenosines (As), at least 200 As, at least 250 As, at least 300 As, at least 400 As, or at least 500 As, such as about 150, 160, 170, 180, 190, 200, 250, 300, 350, 400, 450, or 500 As.

11. Non-Natural Nucleotides/Nucleosides

In some examples, the mRNA includes non-natural nucleotides or ribonucleotides, such as a nucleotide or ribonucleotide analog. In one example, the analogue is a 2′-O-methyl-substituted RNA, locked nucleic acid (LNA) or bridged nucleic acid (BNA), morpholino, or peptide nucleic acid (PNA). These oligonucleotides have a different backbone sugar or, in the case of PNA, an amino acid residue in place of the ribose phosphate, they still bind to RNA or DNA according to Watson and Crick pairing, but are immune to nuclease activity.

In one example, the mRNA includes one or more of isoguanine and isocytosine.

In one example, the mRNA includes pseudouridine (Ψ) and/or 5-methylcytidine (m5C) (e.g., in place of the corresponding U or C canonical nucleosides).

In one example, the mRNA includes pseudouridine (Ψ) in place of the corresponding U nucleosides.

12. 5′-UTR and 3′-UTR

mRNA molecules often have regions of differing sequence located before the translation start codon and after the translation stop codon that are not translated. These regions, termed the five prime untranslated region (5′ UTR) and three prime untranslated region (3′ UTR), respectively, can affect mRNA stability, mRNA localization, and translational efficiency of the mRNA to which they are joined. Certain 5′ and 3′ UTRs, such as those for alpha and beta globins, improve mRNA stability and expression of mRNAs. Thus, in some examples, the mRNAs provided herein include a 5′ UTR and/or a 3′ UTR that results in greater mRNA stability and higher expression of the mRNA in the cells (e.g., an alpha globin or a beta globin 5′ UTR and/or 3′ UTR; e.g., a Xenopus or human alpha globin or a beta globin 5′ UTR and/or 3′ UTR (e.g., one comprising at least 80%, at least 90%, at least 95%, at least 99% or 100% sequence identity to SEQ ID NO: 28, 29, 30 or 31), or, e.g., a tobacco etch virus (TEV) 5′ UTR). However, other 5′ and 3′ UTRs in the human genome and other genomes can be used. A naturally occurring mRNA of interest may include its own 5′ and/or 3′-UTR. The 3′-UTR sequence immediately follows the translation termination codon and appears before the poly-A tail. Thus, for example, if the mRNA is a human gp91^{phox} mRNA, the 3′ UTR can be the 3′-UTR of human gp91^{phox} mRNA. The length of the 3′-UTR can vary, and in some examples is at least 60 nt, at least 100 nt, at least 200 nt, at least 500 nt, at least 800 nt, at least 1000 nt, at least 2500 nt or at least 4000 nt, such as 60 to 4000, 100 to 1000, or 200 to 1500 nt. Exemplary 3′-UTRs are shown in SEQ ID NO: 30 and 31, and include one comprising at least 80%, at least 90%, at least 95%, at least 99% or 100% sequence identity to SEQ ID NO: 30 or 31. Exemplary 5′-UTRs are shown in SEQ ID NO: 28 and 29, and include one comprising at least 80%, at least 90%, at least 95%, at least 99% or 100% sequence identity to SEQ ID NO: 28 or 29, and can include a Kozak sequence (such as one comprising at least 80%, at least 90%, at least 95%, at least 99% or 100% sequence identity to SEQ ID NO: 32).

B. Introduction of mRNA into Immune Cells

mRNAs that encode a protein missing or defective in a PID subject can be introduced into granulocytes, NK cells, and/or lymphocytes (such as an apheresis product), thereby generating recombinant granulocytes, recombinant NK cells, and/or recombinant lymphocytes. In one example, naked nucleic acid molecules are used.

Blood, apheresis product, leukepheresis produce, isolated granulocytes (such as neutrophils), isolated NK cells, and/or isolated lymphocytes, obtained from the subject can be used directly for transfection. However, in some examples such cells are incubated in a culturing medium in a culture apparatus for a period of time or until the cells reach sufficient number before passing the cells to another culture apparatus. The culturing apparatus can be of any culture apparatus commonly used for culturing cells in vitro. In one example, the level of confluency of the cells is greater than 70%, or greater than 90% before passing the cells to another culture apparatus. A period of time can be any time suitable for the culture of cells in vitro. The culturing medium may be replaced during the culture of the cells. The cells are then harvested from the culture apparatus. The cells can be used immediately or they can be cryopreserved (for example in the presence of DMSO) and stored for use at a later time.

Blood, apheresis product, leukepheresis produce, isolated granulocytes (such as neutrophils), isolated NK cells, and/or isolated lymphocytes, to be transfected can be grown in culture. Culture media typically contains a variety of essential components required for cell viability, including inorganic salts, carbohydrates, hormones, essential amino acids, vitamins, and the like. In some embodiments, RPMI is used as a culture medium. Additional additives can be used, such as glutamine, heparin, sodium bicarbonate, serum and/or N2 supplement. The pH of the culture medium is typically between 6-8, such as about 7, for example about 7.4. Cells can be cultured at a temperature between 30-40° C., such as between 35-38° C., such as between 35-37° C., for example at 37° C.

Methods for introducing nucleic acid molecules, which include the desired mRNA, into granulocytes, NK cells, and/or lymphocytes (such as an apheresis product) in culture include chemical and physical methods. Chemical methods include liposome-based gene transfer or lipofection, lipid nanoparticles (LNPs), calcium phosphate-mediated gene transfer, DEAE-dextran transfection techniques, and polyethyleneimine (PEI)-mediated delivery. Physical methods include ballistic gene transfer (introduces particles coated with nucleic acid molecules into cells), microinjection, and nucleofection. In a specific example, granulocytes, NK cells, and/or lymphocytes (such as an apheresis product) are electroporated to allow entry of the mRNAs into the cells. For example, the electroporation can be performed using a GMP-compliant MaxCyte Biosystems (for example at room temperature). In some examples, at least 100 ug/ml of mRNA is used for the transfection, such as at least 200 ug/ml of mRNA, at least 300 ug/ml of mRNA, at least 400 ug/ml of mRNA, or at least 500 ug/ml of mRNA, such as 200 to 400 ug/ml of mRNA, for example with cells at a concentration of at least 1×10⁸ cells/ml, at least 2×10⁸ cells/ml, at least 3×10⁸ cells/ml, at least 4×10⁸ cells/ml, at least 5×10⁸ cells/ml, at least 6×10⁸ cells/ml, at least 7×10⁸ cells/ml, or at least 7.5×10⁸ cells/ml, such 5-7.5×10⁸ cells/ml. In some examples, at least 50 million cells/ml are transfected, such as at least 100 million cells/ml, at least 200 million cells/ml, at least 500 million cells/ml or at least 750 million cells/ml.

Following transfection, cells can be incubated at 37° C., for example cultured at 5-7×10⁶ cells/mL.

In some examples, following transfection, cells are analyzed for the presence of the mRNA(s) introduced into the cell, for example to determine if the cell expresses functional protein(s) corresponding to the mRNA(s) introduced. For example, the cells can be analyzed for NADPH oxidase activity, for example measuring using a flow cytometric dihydrorhodamine assay, or chemiluminescence for quantitative reactive oxidative species production. In one example, the cells can be analyzed for NKG2D expression, or transformed NK cells can be analyzed for cell killing activity (e.g., if an MAGT1 mRNA was introduced).

III. Administration of Recombinant Immune Cells into a Subject

The recombinant autologous granulocytes (e.g., recombinant autologous neutrophils), recombinant autologous NK cells, and/or recombinant autologous lymphocytes, which include one or more mRNAs that express a missing or defective protein in the subject with PID, can be introduced, that is administered or transplanted, into a subject, such as a subject with a PID. In some examples, the subject with PID has a chronic infection. In some examples, the subject is treated prior to receiving a HSC or bone marrow transplant. In some examples, the subject with PID has autoimmune disease, such as Hashimoto's thyroiditis, pernicious anemia, inflammatory bowel disease (Crohn's disease and ulcerative colitis), psoriasis, renal, pulmonary, and hepatic fibroses, Addison's disease, type I diabetes, systemic lupus erythematosus, dermatomyositis, Sjogren's syndrome, multiple sclerosis, myasthenia gravis, Reiter's syndrome, rheumatoid arthritis, or Grave's disease. Thus, transplantation of the recombinant autologous granulocytes and/or recombinant autologous lymphocytes can be used to treat patients with PID, such as one with a chronic infection, acute infection, or autoimmunity.

In some examples therapeutically effective amounts include at least 1×10⁶, at least 2×10⁶, at least 3×10⁶, at least 4×10⁶, at least 5×10⁶, at least 6×10⁶, at least 7×10⁶, at least 8×10⁶, at least 9×10⁶, at least 1×10⁷, at least 2.5×10⁷, at least 5×10⁷, at least 1×10⁸, at least 2.5×10⁸, or at least 5×10⁸ of recombinant autologous granulocytes, recombinant autologous NK cells, and/or recombinant autologous lymphocytes. Such amounts can be introduced into the recipient subject, for example by injection, such as intravenously.

In some examples, multiple separate therapeutic doses of recombinant autologous granulocytes, recombinant autologous NK cells, and/or recombinant autologous lymphocytes are administered to the subject. For example, the recombinant autologous granulocytes, recombinant autologous NK cells, and/or recombinant autologous lymphocytes can be administered to the subject at least twice, at least 5 times, at least 10 times, at least 20 times, at least 40 times, at least 50 times, at least 75 times, at least 100, or at least 500 different times. In some examples, the subject receives such recombinant cells over the course of their entire life.

A. Subjects

In some examples, the subject receiving the recombinant granulocytes and/or recombinant lymphocytes (which in some examples are autologous) can have a PID, such as one disclosed herein. The subject can be a mammal, such as a human, or veterinary subject. In some examples the subject is a pediatric subject (e.g., less than one year old), child (e.g., less than 18 years old), or an adult (e.g., at least 18 years old).

In some examples, the subject has an autoimmune disease as a result of a PID, such as Hashimoto's thyroiditis, pernicious anemia, inflammatory bowel disease (Crohn's disease and ulcerative colitis), psoriasis, renal, pulmonary, and hepatic fibroses, Addison's disease, type I diabetes, systemic lupus erythematosus, dermatomyositis, Sjogren's syndrome, multiple sclerosis, myasthenia gravis, Reiter's syndrome, rheumatoid arthritis, or Grave's disease. Thus, some examples, such autoimmune diseases are treated with the disclosed methods.

In some examples, the subject has an acute or chronic infection.

B. Infections

In some examples, the subject with PID has an acute or chronic infection, such as an acute or chronic bacterial, viral, fungal, or parasitic infection. Such infections can be treated with the disclosed methods.

In some examples, the subject has a Gram-positive or Gram-negative bacterial infection, such as one or more of: Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeum and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (e.g., Klebsiella pneumoniae, Klebsiella granulomatis and Klebsiella oxytoca), Lactobacillus sp., Listeria sp. (e.g., Listeria monocytogenes), Leptospira interrogans, Legionella pneumophila, Leptospira interrogans, Peptostreptococcus sp., Moraxella catarrhalis, Morganella sp., Mobiluncus sp., Micrococcus sp., Mycobacterium sp. (e.g., Mycobacterium leprae, Mycobacterium tuberculosis, Mycobacterium intracellulare, Mycobacterium avium, Mycobacterium bovis, and Mycobacterium marinum), Mycoplasm sp. (e.g., Mycoplasma pneumoniae, Mycoplasma hominis, and Mycoplasma genitalium), Nocardia sp. (e.g., Nocardia asteroides, Nocardia cyriacigeorgica and Nocardia brasiliensis), Neisseria sp. (e.g., Neisseria gonorrhoeae and Neisseria meningitidis), Pasteurella multocida, Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Pseudomonas cepacia, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (s e.g., s Salmonella enterica, Salmonella typhi, Salmonella paratyphi, Salmonella enteritidis, Salmonella cholerasuis and Salmonella typhimurium), Serratia sp. (e.g., Serratia marcesans and Serratia liquifaciens), Shigella sp. (e.g., Shigella dysenteriae, Shigella flexneri, Shigella boydii and Shigella sonnei), Staphylococcus sp. (e.g., Staphylococcus aureus, Staphylococcus epidermidis, Staphylococcus hemolyticus, Staphylococcus saprophyticus), Streptococcus sp. (such as Streptococcus pneumoniae (e.g., chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (e.g., Treponema carateum, Treponema petenue, Treponema pallidum and Treponema endemicum, Tropheryma whippelii, Ureaplasma urealyticum, Veillonella sp., Vibrio sp. (e.g., Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) or Xanthomonas maltophilia, among others.

In some examples, the subject has a positive-strand RNA viral infection, such as infection by one or more of a: Picornavirus (e.g., Aphthovirus [for example foot-and-mouth-disease virus (FMDV)], Cardiovirus; Enterovirus (e.g., Coxsackie viruses, Echoviruses, Enteroviruses, Rhinovirus and Polioviruses); Hepatovirus (e.g., Hepatitis A, B or C virus); Togavirus (e.g., rubella; alphaviruses (such as Western equine encephalitis virus, Eastern equine encephalitis virus, and Venezuelan equine encephalitis virus)); Flavivirus (e.g., Dengue virus, West Nile virus, and Japanese encephalitis virus); Calciviridae (e.g., Norovirus and Sapovirus); or Coronavirus (e.g., human coronavirus 229E, OC43, NL63, HKU1, SARS coronaviruses, and Middle East respiratory syndrome coronavirus).

In some examples, the subject has a negative-strand RNA viral infection, such as infection by one or more of an Orthomyxyovirus (e.g., influenza virus), Rhabdovirus (e.g., Rabies virus), or Paramyxovirus (e.g., measles virus, respiratory syncytial virus, and parainfluenza viruses).

In some examples, the subject has a DNA viral infection, such as infection by one or more of a: Herpesvirus (e.g., Varicella-zoster virus, for example the Oka strain; cytomegalovirus; Herpes simplex virus (HSV) types 1 and 2, and Epstein-Barr virus), Adenoviruses (e.g., Adenovirus type 1 and Adenovirus type 41), Poxviruses (e.g., Vaccinia virus), and Parvoviruses (e.g., Parvovirus B19).

In some examples, the subject has a retroviral infection, such as infection by one or more of: human immunodeficiency virus type 1 (HIV-1), such as subtype C; HIV-2; equine infectious anemia virus; feline immunodeficiency virus (FIV); feline leukemia viruses (FeLV); simian immunodeficiency virus (SIV); or avian sarcoma virus.

In some examples, the subject has an acute or chronic infection by Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, or Zika virus

In some examples, the subject has an infection with a protozoa, nemotode, or fungi. Exemplary protozoa that may infect a subject treated herein include, but are not limited to, Plasmodium (e.g., Plasmodium falciparum: to diagnose malaria), Leishmania, Acanthamoeba, Giardia, Entamoeba, Cryptosporidium, Isospora, Balantidium, Trichomonas, Trypanosoma (e.g., Trypanosoma brucei), Naegleria, and Toxoplasma. Exemplary fungi include, but are not limited to, Aspergillus sp. (including Aspergillus fumigatus), Candida sp., (such as Candida albicans), C. neoformans, C. gattii, Coccidioides sp., Coccidiodes immitis, Trichophyton sp., Microsporum sp., Epidermophyton sp., Tinea sp., and Blastomyces dermatitidis.

In some examples, the subject has CGD and a Staphylococcus, Burkholderia, Nocardia, Serratia, Klebsiella, and/or Aspergillus infection.

C. PIDs

PIDs that can be treated with the disclosed methods include any PID resulting from a genetic defect. In some examples, the PID is caused by a loss of function, that is, the protein is not produced, is deficient, or is defective. PIDs may result from a single genetic defect, but can be multifactorial. PIDs may be caused by recessive or dominant inheritance.

Thus, the disclosed methods can be used to provide an mRNA encoding the missing or defective protein. In some examples, the subject who receives the recombinant granulocytes and/or recombinant lymphocytes has a PID. In some examples, the subject also as an acute or chronic infection, such as a bacterial, fungal, and/or viral infection. In some examples, the subject has autoimmune disease as a result of a PID.

PIDs weaken the immune system, allowing repeated infections and other health problems to occur more easily. Examples of primary immunodeficiency diseases and their corresponding mutations include those listed in Al-Herz et al., Frontiers in Immunology, volume 5, article 162, Apr. 22, 2014, herein incorporated by reference. Specific examples are provided in Table 1.

TABLE 1
Exemplary PIDs and corresponding mutations

| | Genetic | Exemplary | |
|---|---|---|---|
| | Defect/Missing | mRNA/coding | Exemplary |
| Disease | Protein | Sequence | Infections |
| Chronic granulomatous | NADPH oxidase, | SEQ ID NOS: 1, | Staphylococcus |
| disease (CGD) | one of the following | 4, 6, 8, 10, 12, | aureus, |
| | five subunits | 14, 16, 20, 22, 24 | Serratia |
| | gp91phox | | marcescens, |
| | p47phox | | Burkholderia |
| | p67phox | | cepacia |
| | p22phox | | complex, |
| | p40phox | | Listeria, E. |
| | | | coli, |
| | | | Klebsiella, |
| | | | Pseudomonas |
| | | | cepacia, |
| | | | Nocardia, |
| | | | Aspergillus |
| X-linked immunodeficiency | MAGT1 | SEQ ID NO: 2, | Epstein-Barr |
| with magnesium defect, | | 18 | virus (EBV) |
| Epstein-Barr Virus Infection | | | |
| and Neoplasia (XMEN) | | | |
| Autoimmune | TNFRSF6/CD95 | | |
| lymphoproliferative | CASP10, CASP8, | | |
| syndrome (ALPS) (FAS, | FADD, | | |
| FASLG, caspase 10, | PRKCD | | |
| caspase 8, FADD | | | |
| deficiency, PRKC8 | | | |
| deficiency) | | | |
| Autoimmune polyglandular | Autoimmune | | Candida |
| syndrome type 1 (APS-1) | regulator (AIRE) | | |
| BENTA disease | CARD11 | | |
| Caspase 8 deficiency state | Casp8 | | Viral |
| (CEDS) | | | infections |
| CARD9 deficiency | CARD9 | | Candida |
| common variable | TNFRSF13B | | bacterial or |
| immunodeficiency (CVID) | | | viral infections |
| | | | of the upper |
| | | | airway, |
| | | | sinuses, and |
| | | | lungs |
| Congenital Neutropenia | | | |
| Syndromes | | | |
| CTLA4 Deficiency | CTLA4 | 26 | |
| DOCK8 Deficiency | DOCK8 | | recurrent viral |
| | | | infections of |
| | | | the skin and |
| | | | respiratory |
| | | | system |
| GATA2 Deficiency | GATA2 | | |
| Glycogen storage disease | SLC37A4 or | | |
| type 1b | “G6PT1”, the G6P | | |
| | transporter | | |
| Glycosylation Disorders | | | |
| With Immunodeficiency | | | |
| Hyper-Immunoglobulin E | STAT3 | | recurrent |
| Syndrome (HIES) | | | bacterial |
| | | | infections of |
| | | | the skin and |
| | | | lungs |
| Hyper-Immunoglobulin M | CD40 ligand | | respiratory |
| (Hyper-IgM) Syndromes | | | infections, |
| | | | cryptococcal |
| | | | infections |
| Interferon Gamma, | IFN-gamma, IL-12, | | |
| Interleukin 12, Interleukin | IL-23 | | |
| 23 Deficiencies | | | |
| Leukocyte Adhesion | ITGB2 | | |
| Deficiency (LAD) | | | |
| LRBA Deficiency | LRBA | | |
| PI3 Kinase Disease | PI3 Kinase | | |
| PLCG2-associated Antibody | PLCG2 | | |
| Deficiency and Immune | | | |
| Dysregulation (PLAID) | | | |
| Purine nucleoside | PNP | | |
| phosphorylase (PNP) | | | |
| deficiency | | | |
| Severe Combined | adenosine deaminase | | Candida, |
| Immunodeficiency (SCID) | (ADA), RAG1, | | Pneumocystis |
| (such as ADA SCID, T-B+ | RAG2, IL-2RG, | | jirovecii |
| SCID, T-B− SCID, IL-7 | JAK3, IL-2, -4, -7, -9, -15 | | |
| SCID) | or -21 | | |
| Wiskott-Aldrich Syndrome | WAS | | recurrent |
| (WAS) | | | bacterial and |
| | | | fungal |
| | | | infections |
| X-Linked | XLA (Bruton | | infections of |
| Agammaglobulinemia | tyrosine kinase or | | the ears, throat, |
| (XLA) | BTK) located on the | | lungs, and |
| | X chromosome | | sinuses |
| X-Linked | SH2D1A (SAP | | EBV |
| Lymphoproliferative | protein) | | |
| Disease (XLP) | | | |

In some examples, the subject has one of the following PIDs resulting in combined T and B-cell immunodeficiencies: T−/B+ SCID (γc deficiency, JAK3 deficiency, interleukin 7 receptor chain α deficiency, CD45 deficiency, CD36/CD3F deficiency), T−/B− SCID (RAG 1/2 deficiency, DCLREC deficiency, adenosine deaminase (ADA) deficiency, reticular dysgenesis), Omenn syndrome, 4.DNA ligase type IV deficiency, Cernunnos deficiency, CD40 ligand deficiency, CD40 deficiency, Purine nucleoside phosphorylase (PNP) deficiency, CD37 deficiency, CD8 deficiency, 1ZAP-70 deficiency, Ca++ channel deficiency, MHC class I deficiency, MHC class II deficiency, Winged helix deficiency, CD25 deficiency, STAT5b deficiency, Itk deficiency, DOCK8 deficiency, Activated PI3K Delta Syndrome, MALT1 deficiency, BCL10 deficiency, or CARD11 deficiency.

In some examples, the subject has one of the following PIDs resulting in an antibody deficiency: X-linked agammaglobulinemia (btk deficiency, or Bruton's agammaglobulinemia), μ-Heavy chain deficiency, 15 deficiency, Igα deficiency, BLNK deficiency, thymoma with immunodeficiency, common variable immunodeficiency (CVID), ICOS deficiency, CD19 deficiency, TACI (TNFRSF13B) deficiency, BAFF receptor deficiency, Hyper-IgM syndromes, heavy chain deletions, kappa chain deficiency, isolated IgG subclass deficiency, IgA with IgG subclass deficiency, selective immunoglobulin A deficiency, or Transient hypogammaglobulinemia of infancy (THI).

In some examples, the subject has one of the following PIDs: Wiskott-Aldrich syndrome, ataxia-telangiectasia, ataxia-like syndrome, Nijmegen breakage syndrome, Bloom syndrome, DiGeorge syndrome (when associated with thymic defects), cartilage-hair hypoplasia, Schimke syndrome, Hermansky-Pudlak syndrome type 2, Hyper-IgE syndrome, chronic mucocutaneous candidiasis, hepatic venoocclusive disease with immunodeficiency (VODI), or XL-dyskeratosis congenita (Hoyeraal-Hreidarsson syndrome).

In some examples, the subject has one of the following PIDs: Chediak-Higashi syndrome, Griscelli syndrome type 2, perforin deficiency, UNC13D deficiency, syntaxin 11 deficiency, X-linked lymphoproliferative syndrome, Autoimmune lymphoproliferative syndrome: type 1a (CD95 defects), type 1b (Fas ligand defects), type 2a (CASP10 defects), type 2b (CASP8 defects); APECED (autoimmune polyendocrinopathy with candidiasis and ectodermal dystrophy); IPEX (immunodysregulation polyendocrinopathy enteropathy X-linked syndrome); or CD25 deficiency.

In some examples, the subject has one of the following PIDs related to defects in phagocyte number/function: Severe Congenital Neutropenia: due to ELA2 deficiency (with myelodysplasia), Severe Congenital Neutropenia: due to GFIl deficiency (with T/B lymphopenia), Kostmann syndrome, Neutropenia with cardiac and urogenital malformations, Glycogen storage disease type 1b, Cyclic neutropenia, X-linked neutropenia/myelodysplasia, P14 deficiency, Leukocyte adhesion deficiency type 1, Leukocyte adhesion deficiency type 2, Leukocyte adhesion deficiency type 3, RAC2 deficiency (Neutrophil immunodeficiency syndrome), Beta-actin deficiency, Localized juvenile periodontitis, Papillon-Lefevre syndrome, Specific granule deficiency, Shwachman-Diamond syndrome, Chronic granulomatous disease: X-linked, Chronic granulomatous disease: autosomal (CYBA), Chronic granulomatous disease: autosomal (NCF1), Chronic granulomatous disease: autosomal (NCF2), IL-12 and IL-23 β1 chain deficiency, IL-12p40 deficiency, 2Interferon γ receptor 1 deficiency, Interferon γ receptor 2 deficiency, STAT1 deficiency (2 forms), AD hyper-IgE, 2AR hyper-IgE, or pulmonary alveolar proteinosis.

In some examples, the subject has one of the following PIDs related to defects in innate immunity: Hypohidrotic ectodermal dysplasia (NEMO deficiency, IKBA deficiency); EDA-ID, IRAK-4 deficiency, MyD88 deficiency, Epidermodysplasia verruciformis, Herpes simplex encephalitis, chronic mucocutaneous candidiasis, or Trypanosomiasis.

In some examples, the subject has one of the following PIDs related to an autoinflammatory disorder: Familial Mediterranean fever, or TNF receptor associated periodic syndrome (TRAPS), Hyper-IgD syndrome (HIDS), CIAS1-related diseases (Muckle-Wells syndrome, Familial cold autoinflammatory syndrome, Neonatal onset multisystem inflammatory disease), PAPA syndrome (pyogenic sterile arthritis, pyoderma gangrenosum, acne), Blau syndrome, Chronic recurrent multifocal osteomyelitis and congenital dyserythropoietic anemia (Majeed syndrome), or DIRA (deficiency of the IL-1 receptor antagonist).

In some examples, the subject has one of the following PIDs related to a complement deficiency: C1q deficiency (lupus-like syndrome, rheumatoid disease, infections), C1r deficiency (idem), C1s deficiency, C4 deficiency (lupus-like syndrome), C2 deficiency (lupus-like syndrome, vasculitis, polymyositis, pyogenic infections), C3 deficiency (recurrent pyogenic infections), C5 deficiency (Neisserial infections, SLE), C6 deficiency (idem), C7 deficiency (idem, vasculitis), C8a deficiency, C8b deficiency, C9 deficiency (Neisserial infections), C1-inhibitor deficiency (hereditary angioedema), Factor I deficiency (pyogenic infections), Factor H deficiency (haemolytic-uraemic syndrome, membranoproliferative glomerulonephritis), Factor D deficiency (Neisserial infections), Properdin deficiency (Neisserial infections), MBP deficiency (pyogenic infections), MASP2 deficiency, Complement receptor 3 (CR3) deficiency, Membrane cofactor protein (CD46) deficiency, Membrane attack complex inhibitor (CD59) deficiency, Paroxysmal nocturnal hemoglobinuria, or Immunodeficiency associated with ficolin 3 deficiency.

Administration of the recombinant autologous granulocytes, recombinant autologous NK cells, and/or recombinant autologous lymphocytes can be used to treat any of these disorders. Treatment does not require 100% removal of all characteristics of the disorder, but can be a reduction in such.

In one example the disclosed methods reduce the symptoms of an infection in the recipient subject (such as one or more of fever, large tender lymph nodes, throat inflammation, a rash, headache, sores of the mouth, nausea, vomiting, diarrhea, weight loss, viral load, ulcer size, size of infiltrate on imaging of lungs, brain, skin etc., blood parameters such as white cell count, inflammatory markers such as erythrocyte sedimentation rate, and C-reactive protein, and the like) for example a reduction of at least 10%, at least 20%, at least 50%, at least 70%, or at least 90% (as compared to no administration of the recombinant autologous granulocytes, recombinant autologous NK cells, and/or recombinant autologous lymphocytes).

In one example the disclosed methods increase the number of immune cells expressing the missing protein (e.g., having functional NADPH oxidase activity), increase detectable protein expression (that was previously absent in the subject), increase the number of regulatory cells, increase immune competence (e.g., immunoglobulin class switch), increase the number of immune memory cells, and/or increase or improve the clinical status (e.g., weight, appetite, resolution of fevers), in the PID recipient subject, for example an increase of at least 5%, at least 10%, at least 20%, at least 50%, at least 70%, at least 90%, at least 100%, at least 200%, at least 500% or at least 1000% (as compared to no administration of the recombinant autologous granulocytes, recombinant autologous NK cells, and/or recombinant autologous lymphocytes).

D. Additional Treatments

In some examples, in addition to receiving recombinant autologous granulocytes, recombinant autologous NK cells, and/or recombinant autologous lymphocytes, the treated subjects will receive additional therapy or treatment.

In one example, the subject is administered a therapeutically effective amount of an antibiotic, such as one or more of: chlortetracycline, amikacin, gentamicin, kanamycin, neomycin, netilmicin, tobramycin, paromomycin, streptomycin, spectinomycin, Geldanamycin, Herbimycin, Rifaximin, Loracarbef, Ertapenem, Doripenem, Imipenem/Cilastatin, Meropenem, Cefadroxil, Cefazolin, Cefalexin, Cefaclor, Cefprozil, Cefuroxime, Cefixime, Cefdinir, Cefditoren, Cefoperazone, Cefotaxime, Cefpodoxime, Ceftazidime, Ceftibuten, Ceftriaxone, Cefepime, Ceftaroline fosamil, Ceftobiprole, Teicoplanin, Vancomycin, Telavancin, Dalbavancin, Oritavancin, Clindamycin, Lincomycin, Daptomycin, Azithromycin, Clarithromycin Erythromycin, Roxithromycin, Telithromycin, Spiramycin, Aztreonam, Furazolidone, Nitrofurantoin, Linezolid, Posizolid, Radezolid, Torezolid, Amoxicillin, Ampicillin, Azlocillin, Dicloxacillin, Flucloxacillin, Mezlocillin, Methicillin, Nafcillin, Oxacillin, Penicillin G, Penicillin V, Piperacillin, Penicillin G, Temocillin, Ticarcillin, Amoxicillin/clavulanate, Ampicillin/sulbactam, Piperacillin/tazobactam, Ticarcillin/clavulanate, Bacitracin, Colistin, Polymyxin B, Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, Temafloxacin, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole) (TMP-SMX), Sulfonamidochrysoidine, Demeclocycline, Doxycycline, Metacycline, Minocycline, Oxytetracycline, Tetracycline, Clofazimine, Dapsone, Capreomycin, Cycloserine, Ethambutol, Ethionamide, Isoniazid, Pyrazinamide, Rifampicin, Rifabutin, Rifapentine, Streptomycin, Arsphenamine, Chloramphenicol, Fosfomycin, Fusidic acid, Metronidazole, Mupirocin, Platensimycin, Quinupristin/Dalfopristin, Thiamphenicol, Tigecycline, Tinidazole, Trimethoprim, or combinations thereof.

In one example, the subject is administered a therapeutically effective amount of an antiviral, such as one or more of: Abacavir, Acyclovir (Aciclovir), Adefovir, Amantadine, Amprenavir(Agenerase), Ampligen, Arbidol, Atazanavir, Atripla (fixed dose drug), Balavir, Cidofovir, Combivir (fixed dose drug), Dolutegravir, Darunavir, Delavirdine, Didanosine, Docosanol, Edoxudine, Efavirenz, Emtricitabine, Enfuvirtide, Entecavir, Ecoliever, Famciclovir, Fixed dose combination (antiretroviral), Fomivirsen, Fosamprenavir, Foscarnet, Fosfonet, Fusion inhibitor, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nitazoxanide, Nucleoside analogues, Norvir, Oseltamivir (Tamiflu), Peginterferon alfa-2a, Penciclovir, Peramivir, Pleconaril, Podophyllotoxin, Protease inhibitor (pharmacology), Raltegravir, Reverse transcriptase inhibitor, Ribavirin, Rimantadine, Ritonavir, Pyramidine, Saquinavir, Sofosbuvir, Stavudine, Synergistic enhancer (antiretroviral), Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), Zidovudine, or combinations thereof.

In one example, the subject is administered a therapeutically effective amount of an antifungal, such as one or more of: Amphotericin B, Candicidin, Filipin, Hamycin, Natamycin, Nystatin, Rimocidin, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Albaconazole, Efinaconazole, Epoxiconazole, Fluconazole, Isavuconazole, Itraconazole, Posaconazole, Propiconazole, Ravuconazole, Terconazole, Voriconazole, Abafungin, amorolfin, butenafine, naftifine, terbinafine, Anidulafungin, Caspofungin, Micafungin, Aurones, Benzoic acid, Ciclopirox, Flucytosine or 5-fluorocytosine, Griseofulvin, Haloprogin, Tolnaftate, Undecylenic acid, Crystal violet, Balsam of Peru, Orotomide, Miltefosine, or combinations thereof.

In one example, the subject is administered an allogenic or autologous HSC transplant or a bone marrow transplant, for example following administration of the recombinant autologous granulocytes and/or recombinant autologous lymphocytes.

Immunomodulation, the subject is administered a therapeutically effective amount of an interferon, such as interferon gamma-1b.

In some examples, combinations of these additional therapies are administered.

In one example, the subject has CGD and is also administered an antibiotic such as trimethoprim-sulfamethoxazole, an antifungal such as itraconazole, voriconazole, isovuconazole, terbinafine, amphotericin, or combinations thereof. In one example, the subject has CGD and is also administered a therapeutically effective amount of an interferon, such as interferon gamma-1b.

In one example, the subject has XMEN and is also administered magnesium supplementation (e.g., oral magnesium threonate supplements).

In one example, the subject has CTLA4 deficiency and is also administered sirolimus or orencia.

### Recombinant Immune Cells

The present disclosure also provides recombinant autologous immune cells, such as granulocytes (e.g., neutrophils), NK cells, and lymphocytes, which include one or more non-native/exogenous mRNA molecules (which can be naked, or part of a vector, such as a plasmid or viral vector) encoding an active form of the protein(s) which is deficient or defective in a PID subject. Also provided are compositions that include such cells, for example as well as a pharmaceutically acceptable carrier (such as water or saline), a growth media (such as DMEM or RPMI), or a cryo-preservative (such as DMSO). In some examples, the composition is lyophilized, and can be reconstituted (e.g., with water or saline) before administration into a patient. In some examples, the composition is frozen, and can be thawed before administration into a patient.

Exemplary mRNAs that can be present in the recombinant cells are provided herein. In one example, the mRNA molecule encodes a protein deficient or defective in a subject with a PID, such as one listed in Table 1. For example, the mRNA can encode CD18/beta 2 integrin, MagT1, CTLA4, FoxP3, CD40Ligand, CARD9 (caspase recruitment domain-containing protein 9), or CARD11 (caspase recruitment domain-containing protein 11).

Exemplary mRNAs provided herein that can be present in the recombinant cells are shown in SEQ ID NOS: 1, 2, 4, 6, 8, 10, 12, 14, 16, 18, 20, 22, 24 and 26. Thus the disclosure provides isolated recombinant autologous immune cells, such as granulocytes (e.g., neutrophils), NK cells, and lymphocytes, which include one or more mRNA molecules having at least 60%, at least 70%, at least 75%, at least 80%, at least 85%, at least 90%, at least 95%, at least 97%, at least 98%, at least 99%, or 100% sequence identity to any one of SEQ ID NOS: 1, 2, 4, 6, 8, 10, 12, 14, 16, 18, 20, 22, 24 and 26 (wherein Ts in such sequences can be a uridine in the mRNA sequence, or an unnatural nucleoside such as pseudouridine) as long as they encode a functional protein.

In some examples, the mRNA present in the recombinant autologous immune cells, such as granulocytes (e.g., neutrophils), NK cells, or lymphocytes, encodes a protein that is missing or defective in a CDG patient, such as encodes a native or wild-type CYBA (p22phox), CYBB (gp91phox), NCF1 (p47phox), NCF2 (p67phox), NCF4 (p40phox), or combinations thereof. In some examples, the mRNA encodes a protein having at least 80%, at least 85%, at least 90%, at least 95%, at least 97%, at least 98%, at least 99%, or 100% sequence identity to SEQ ID NO: 5, 7, 9, 11, 13, 15, 17, 21, 23, and 25. In some examples, the portion of the mRNA coding for the protein has at least 60%, at least 70%, at least 75%, at least 80%, at least 85%, at least 90%, at least 95%, at least 97%, at least 98%, at least 99%, or 100% sequence identity to SEQ ID NO: 1, 4, 6, 8, 10, 12, 14, 16, 20, 22, or 24 (wherein an mRNA can have a U or pseudouridine or other unnatural nucleoside in place of the Ts of SEQ ID NO: 1, 4, 6, 8, 10, or 12).

In some examples, the mRNA present in the recombinant autologous immune cells, such as granulocytes (e.g., neutrophils), NK cells, or lymphocytes, encodes a protein that is missing or defective in a XMEN patient, such as encodes a native or wild-type MagT1 protein. In some examples, the mRNA encodes a MagT1 protein having at least 80%, at least 85%, at least 90%, at least 95%, at least 97%, at least 98%, at least 99%, or 100% sequence identity to SEQ ID NO: 3 or 29. In some examples, the portion of the mRNA coding for the protein has at least 60%, at least 70%, at least 75%, at least 80%, at least 85%, at least 90%, at least 95%, at least 97%, at least 98%, at least 99%, or 100% sequence identity to SEQ ID NO: 2 or 18 (wherein an mRNA can have a U or pseudouridine or other unnatural nucleosides in place of the Ts of SEQ ID NO: 2).

In some examples, the mRNA present in the recombinant autologous immune cells, such as granulocytes (e.g., neutrophils), NK cells, or lymphocytes, encodes a protein that is missing or defective in a CTLA4 deficient patient, such as encodes a native or wild-type CTLA4 protein. In some examples, the mRNA encodes a CTLA4 protein having at least 80%, at least 85%, at least 90%, at least 95%, at least 97%, at least 98%, at least 99%, or 100% sequence identity to SEQ ID NO: 27. In some examples, the portion of the mRNA coding for the protein has at least 60%, at least 70%, at least 75%, at least 80%, at least 85%, at least 90%, at least 95%, at least 97%, at least 98%, at least 99%, or 100% sequence identity to SEQ ID NO: 26.

The sequences shown in SEQ ID NOS: 1, 2, 4, 6, 8, 10, 12, can have their Ts replaced with uridines, or an unnatural nucleoside such as pseudouridine, in place of the Ts. Thus, in some examples, the portion of the mRNA coding for the protein has at least 60%, at least 70%, at least 75%, at least 80%, at least 85%, at least 90%, at least 95%, at least 97%, at least 98%, at least 99%, or 100% sequence identity to SEQ ID NOS 1, 2, 4, 6, 8, 10, or 12, but has their Ts replaced with uridines, or an unnatural nucleoside such as pseudouridine.

In some examples, the portion of the mRNA coding for the protein present in the recombinant autologous immune cells, such as granulocytes (e.g., neutrophils), NK cells, or lymphocytes, has at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26. As noted above, a coding sequence shown in SEQ ID NOS 1, 2, 4, 6, 8, 10, or 12, can have their Ts replaced with uridines, or an unnatural nucleoside such as pseudouridine. mRNAs introduced in the disclosed autologous granulocytes, autologous NK cells, and/or autologous lymphocytes (generating recombinant cells) and used in the disclosed methods can include such mRNA coding sequences, and can further include (1) a 5′-end cap, (2) a 3′-end poly-A tail (such as 150 or more As), (3) a 3′-UTR (such as a human or Xenopus beta globin 3′-UTR, such as one having at least 60%, at least 70%, at least 75%, at least 80%, at least 85%, at least 90%, at least 95%, at least 97%, at least 98%, at least 99%, or 100% sequence identity to SEQ ID NO: 30 or 31); (4) a 5′-UTR (such as a human or Xenopus beta globin 5′-UTR, such as one including at least 60%, at least 70%, at least 75%, at least 80%, at least 85%, at least 90%, at least 95%, at least 97%, at least 98%, at least 99%, or 100% sequence identity to SEQ ID NO: 28 or 29) (which can include a Kozak sequence), (5) can be codon optimized for expression in a human cell, and/or (6) include one or more pseudouridines or other unnatural nucleoside in place of one or more uridines (or Ts) (e.g., replace at least 90%, at least 95%, at least 99%, or 100% of all U or Ts).

Thus, in one example, the mRNA present in the recombinant autologous immune cells, such as granulocytes (e.g., neutrophils), NK cells, or lymphocytes, includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26 (wherein the mRNA can have a U or pseudouridine or other unnatural nucleoside in place of the Ts of SEQ ID NO: 1, 2, 4, 6, 8, 10, or 12), and a 5′-end cap. In one example, the mRNA includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26 (wherein the mRNA can have a U or pseudouridine or other unnatural nucleoside in place of the Ts of SEQ ID NO: 1, 2, 4, 6, 8, 10, or 12), and a 3′-end poly-A tail (such as 150 or more As). In one example, the mRNA includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26 (wherein the mRNA can have a U or pseudouridine or other unnatural nucleoside in place of the Ts of SEQ ID NO: 1, 2, 4, 6, 8, 10, or 12), and a 3′-UTR (such as a human or Xenopus beta globin 3′-UTR, such as one having at least 60%, at least 70%, at least 75%, at least 80%, at least 85%, at least 90%, at least 95%, at least 97%, at least 98%, at least 99%, or 100% sequence identity to SEQ ID NO: 30 or 31). In one example, the mRNA includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26 (wherein the mRNA can have a U or pseudouridine or other unnatural nucleoside in place of the Ts of SEQ ID NO: 1, 2, 4, 6, 8, 10, or 12), and a 5′-UTR (such as a human or Xenopus beta globin 5′-UTR, such as one having at least 60%, at least 70%, at least 75%, at least 80%, at least 85%, at least 90%, at least 95%, at least 97%, at least 98%, at least 99%, or 100% sequence identity to SEQ ID NO: 28 or 29) (which can include a Kozak sequence). In one example, the mRNA includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26 (wherein the mRNA can have a U or pseudouridine or other unnatural nucleoside in place of the Ts of SEQ ID NO: 1, 2, 4, 6, 8, 10, or 12), wherein the coding sequence is codon optimized for expression in a human cell. In one example, the mRNA includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26 (wherein the mRNA can have a U or pseudouridine or other unnatural nucleoside in place of the Ts of SEQ ID NO: 1, 2, 4, 6, 8, 10, or 12), wherein one or more uridines (or Ts) are replaced with pseudouridine or other unnatural nucleoside (e.g., least 90%, at least 95%, at least 99%, or 100% of all U or Ts replaced). Combinations of such are also envisioned.

In one example, the mRNA present in the recombinant autologous immune cells, such as granulocytes (e.g., neutrophils), NK cells, or lymphocytes, includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26 (wherein the mRNA can have a U or pseudouridine or other unnatural nucleoside in place of the Ts of SEQ ID NO: 1, 2, 4, 6, 8, 10, or 12), and a 5′-end cap and a 3′-end poly-A tail (such as 150 or more As). In one example, the mRNA includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26 (wherein the mRNA can have a U or pseudouridine or other unnatural nucleoside in place of the Ts of SEQ ID NO: 1, 2, 4, 6, 8, 10, or 12), and a 3′-UTR (such as a human or Xenopus beta globin 3′-UTR, such as one having at least 60%, at least 70%, at least 75%, at least 80%, at least 85%, at least 90%, at least 95%, at least 97%, at least 98%, at least 99%, or 100% sequence identity to SEQ ID NO: 30 or 31) and a 5′-UTR (such as a human or Xenopus beta globin 5′-UTR, such as one having at least 60%, at least 70%, at least 75%, at least 80%, at least 85%, at least 90%, at least 95%, at least 97%, at least 98%, at least 99%, or 100% sequence identity to SEQ ID NO: 28 or 29) (which can include a Kozak sequence). In one example, the mRNA includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26, wherein the coding sequence is codon optimized for expression in a human cell and wherein one or more uridines (or Ts) are replaced with pseudouridine or other unnatural nucleoside (e.g., least 90%, at least 95%, at least 99%, or 100% of all U or Ts replaced). Combinations of such are also envisioned.

In one example, the mRNA present in the recombinant autologous immune cells, such as granulocytes (e.g., neutrophils), NK cells, or lymphocytes, includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26 (wherein the mRNA can have a U or pseudouridine or other unnatural nucleoside in place of the Ts of SEQ ID NO: 1, 2, 4, 6, 8, 10, or 12), and a 5′-end cap, a 3′-end poly-A tail (such as 150 or more As), a 3′-UTR (such as a human or Xenopus beta globin 3′-UTR, such as one having at least 60%, at least 70%, at least 75%, at least 80%, at least 85%, at least 90%, at least 95%, at least 97%, at least 98%, at least 99%, or 100% sequence identity to SEQ ID NO: 30 or 31) and a 5′-UTR (such as a human or Xenopus beta globin 5′-UTR, such as one having at least 60%, at least 70%, at least 75%, at least 80%, at least 85%, at least 90%, at least 95%, at least 97%, at least 98%, at least 99%, or 100% sequence identity to SEQ ID NO: 28 or 29) (which can include a Kozak sequence). In one example, such an mRNA has a coding sequence is codon optimized for expression in a human cell. In one example, such an mRNA has one or more uridines (or Ts) replaced with pseudouridine or other unnatural nucleoside (e.g., least 90%, at least 95%, at least 99%, or 100% of all U or Ts replaced). Combinations of such are also envisioned.

In one example, the mRNA present in the recombinant autologous immune cells, such as granulocytes (e.g., neutrophils), NK cells, or lymphocytes, includes a portion coding for the protein having at least 60%, at least 70%, at least 75%, at least 80%, at least 85%, at least 90%, at least 95%, at least 97%, at least 98%, at least 99%, or 100% sequence identity to SEQ ID NO: 1, 2, 4, 6, 8, 10, 12, 14, 16, 18, 20, 22, 24, or 26 (wherein the mRNA can have a U or pseudouridine or other unnatural nucleoside in place of the Ts of SEQ ID NO: 1, 2, 4, 6, 8, 10, or 12), a 5′-end cap, a 3′-end poly-A tail (such as 150 or more As), a 3′-UTR (such as a human or Xenopus beta globin 3′-UTR, such as one having at least 60%, at least 70%, at least 75%, at least 80%, at least 85%, at least 90%, at least 95%, at least 97%, at least 98%, at least 99%, or 100% sequence identity to SEQ ID NO: 30 or 31), a 5′-UTR (such as a human or Xenopus beta globin 5′-UTR, such as one having at least 60%, at least 70%, at least 75%, at least 80%, at least 85%, at least 90%, at least 95%, at least 97%, at least 98%, at least 99%, or 100% sequence identity to SEQ ID NO: 28 or 29) (which can include a Kozak sequence), has a coding sequence is codon optimized for expression in a human cell, and has one or more uridines (or Ts) replaced with pseudouridine or other unnatural nucleoside (e.g., least 90%, at least 95%, at least 99%, or 100% of all U or Ts replaced).

### Example 1

### Materials and Methods

This example describes the materials and methods used to obtain the results provided in Examples 2-8.

### Subjects

Blood from healthy volunteers and X-CGD patients was obtained after written informed consent. All subjects received 5 daily injections of G-CSF at 10-16 μg/kg. Some patients also receive one dose of pleraxifor (0.24 mg/kg) if stem cells were also being collected. Apheresis products with less than 60% granulocytes underwent elutriation to improve purity.

The feasibility of correcting autologous granulocytes from patients with chronic granulomatous disease by mRNA transfection to provide transient restoration of protein and function was determined. To optimize transfecting mRNA into peripheral blood immune cells by electroporation, leukapheresis or apheresis cell products were collected from adult normal volunteers, and from X-linked gp91phox deficient CGD and autosomal recessive p47-deficient type CGD patients. Apheresis may be performed on CGD patients primarily for the preparation in anticipation of a stem cell transplant or hematopoietic stem cell gene therapy for CGD patients, where a portion of the product was used for this study. Cell products from three each of X-CGD and p47phox deficient CGD patients were used to verify conditions for transfection. A fourth X-CGD patient provided cells for the Cell Manufacture Control validation run, for translation to the downstream manufacture of clinical products.

All data were collected in order to determine the best conditions for the mRNA transfections so as to achieve robust and reproducible outcomes, despite individual patient variability and clinical conditions. The conditions were repeated with cells from multiple individuals (normal volunteers and CGD patients) as well as multiple time points, multiple cell densities and multiple mRNA concentrations that provided important substantiation by repetition under a wide range of conditions. This also allows easy detection of outliers that fall outside of the trends. Outliers have not been excluded from analysis. Furthermore, the experiments have been performed by three different groups at independent sites.

Transfection with mRNA

Granulocytes. Cells were washed with Electroporation Buffer MaxCyte's proprietary electroporation buffer supplemented with 1% human serum albumin (HSA) three times, then resuspended in EP buffer at 5-7.5×10⁸ cells/ml. The cells were mixed well with mRNA, then transfected in appropriate size Processing Assembly (PA)OC-100/400), or a CL-2 processing assembly for samples greater than 10 ml per manufacturer's instructions. Post treatment, cells were incubated for 10-20 minutes at 37° C., then cultured at 5-7×10⁶/mL. Cells are analyzed for viability, protein expression and NADPH oxidase function at indicated times.

Lymphocytes and Natural Killer cells. Leukapheresis products were cryopreserved following collection. Following thaw, cells were rested in RPMI supplemented with fetal bovine serum for two hours, washed with EP buffer two times, resuspended at 5-8×10⁸/ml, mixed thoroughly with relevant mRNA, and electroporated per manufacturer's instructions specific for lymphocytes. Post treatment, cells were incubated for 10-20 minutes at 37° C., then cultured at 5-7×10⁶/ml. Expansion of T cells in leukapheresis products was performed using the T Cell Activation/Expansion Kit per manufacturers' instructions (Miltenyi Biotec).

### Mouse Studies

The animals used were NOD.Cg-Prkdcscid Il2rgtm1Wjl/SzJ mice (The Jackson Laboratory. To determine their chemotactic capability, mRNA-transfected cells were injected via tail vein and thioglycolate was injected i.p. to induce peritonitis. The animals were euthanized 6-8 hours later for peripheral blood and peritoneal lavage for flow cytometric analysis for human cells and gp91^{phox} expression.

### Non-Human Primate Studies

Non-human primates (rhesus macaque) were used to evaluate the safety of infusing increasing numbers of autologous mRNA transfected leukapheresis cells and to identify GFP+ cells from peripheral blood following infusion out to 4-5 days. Since only two animals were used, although one animal underwent the procedure multiple times, a descriptive analysis of the data was provided instead because of the small sample size. Rhesus macaques previously received GFP-transduced rhesus repopulating cells so that they expressed low levels of GFP, to reduce the risk of immune response to GFP from the GFP mRNA transfection and multiple infusions.

Granulocytes were mobilized from previously transplanted rhesus who were tolerant to the Green Fluorescent Protein (GFP) following one dose (15 μg/kg) G-CSF and 1 mg/kg dexamethasone administered 12-24 hours prior to leukapheresis. Leukapheresis products were collected and after GFP mRNA transfection, were reinfused into the monkeys.

In the animal that received three collections and transfusions, cells were administered at 1×10⁷ cells/kg initially, and then 1×10⁸ cells/kg, and then 5×10⁸ cells/kg. Cells were administered at 1-5×10⁷/mL in saline, infused over 15-20 minutes. Chest radiographs were taken prior to and following cell administration. Vital signs, including blood pressure were monitored. Blood was drawn prior to, 30 minutes, 1 hours, 24 hours, then 2, 3, 4 days with an additional day (5) for following infusion to monitor GFP expression in peripheral blood.

### Flow Cytometry

Human hematopoietic cells were detected with anti-human CD45-PE (phycoerythrin), and human gp91^{phox} expression was determined by indirect staining with murine monoclonal antibody 7D5 followed by FITC-conjugated goat anti-mouse immunoglobulin G (IgG) antibody. Antibodies used for the XMEN patient cells included anti-CD3 (UCHT1, Invitrogen or HIT3a), CD4 (OKT4 or RPA-T4), CD8 (RPA-T8 or SK1), CD16 (3G8), CD56 (MEM-188), and CD314 (NKG2D) (1Dl1). Unless otherwise indicated, all antibodies were from Biolegend (San Diego, Calif.). Cells were washed in FACS buffer prior to acquisition on FACS Canto (Argon laser; Becton Dickinson, San Jose, Calif.) and analyses performed using FlowJo software. Intracellular gp91^{phox} stain, p47^{phox} stain, dihydrorhodamine (DHR), and chemiluminescence assays were performed as previously described (De Ravin et al., Sci Transl Med 9:372, 2017; De Ravin et al., Nat Biotechnol 34:424-429, 2016).

### Cytotoxicity Assay

Cryopreserved XMEN leukapheresis cells and normal volunteer peripheral blood mononuclear cells were thawed and transfected on the same day, then analyzed for NKG2D and CD56 expression. The transfected cells were then incubated with K562 target cells labeled with TFL-4 at Effector:Target ratios up to 1 based on CD56+ cell numbers in each prep, spun at 100×g for 5 minutes, followed by incubation at 37° C. for 5 hours. The cells were then stained with Propidium iodide at 20 ng/well), then analyzed by flow cytometry at TFL-4 labeled targets for PI incorporation as a marker of cytoxicity.

### Statistical Analysis

All data is presented with Prism 7 GraphPad program. The experiments conducted were stepwise in the choice of best conditions to arrive at parameters most applicable to clinical manufacture of cell product, and the limited availability of patient cells limit samples sizes for standard statistical analysis. There were significant overlap of experimental parameters to substantiate interpretation across a range of conditions. No pre-processing steps were used to report the data.

For the cytotoxicity assay for NK killing of K562 targets, the controls from three healthy donors were plotted for standard error of mean (SEM).

### Example 2

### Transfection with GFP mRNA

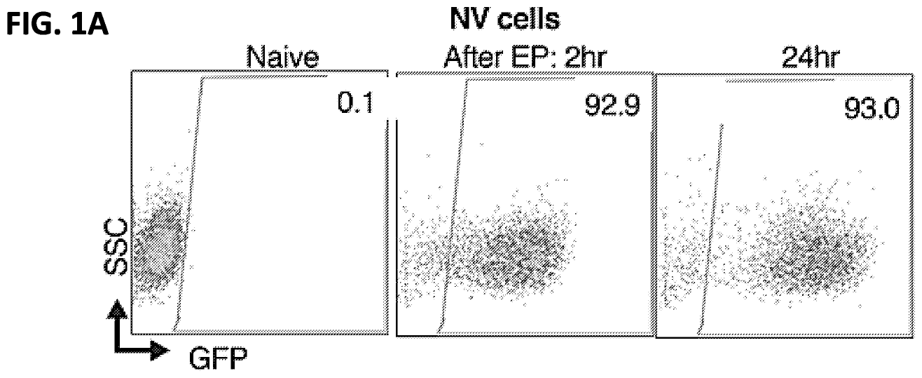

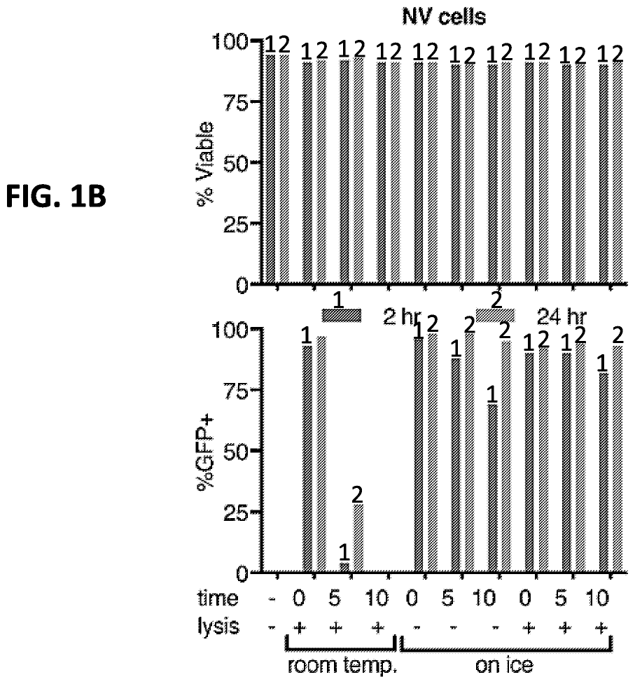

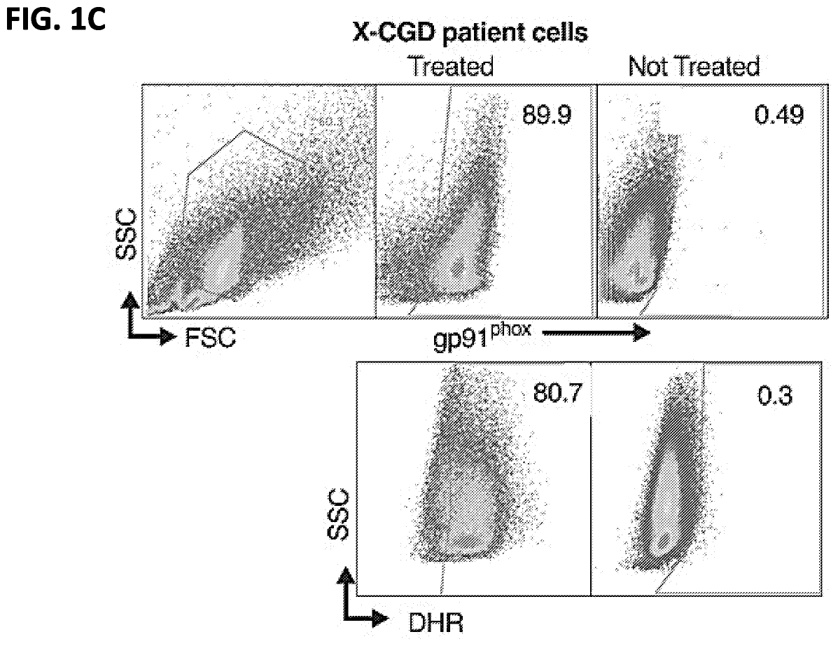

Conditions for mRNA transfection were optimized using adult normal volunteer (NV) granulocytes obtained by leukapheresis by electroporation (MaxCyte GT System) with an mRNA encoding Green Fluorescent Protein (GFP mRNA, CELLSCRIPT, Madison, Wis.) (FIG. 1A).

Since granulocytes are prone to activation and degranulation, the effects of mRNA incubation time and temperature prior to electroporation, and red blood cell lysis on cell viability and transfection efficiency, were evaluated. Incubation of cells with GFP mRNA at room temperature for 5 minutes prior to electroporation decreased GFP expression to baseline, whereas incubation on ice for up to 10 minutes with or without red blood cell lyses resulted in ≥90% GFP expressing cells at 24 hrs with no significant impact on the viability and the transfection efficiency (FIG. 1B).

### Example 3

### Transfection with gp91^{phox} mRNA

The optimized electroporation (EP) conditions determined using GFP mRNA (Example 2), were used to evaluate transfection and expression of gp91^{phox} mRNA in X-CGD patient cells.

X-CGD patients underwent apheresis collection following either a 5-day G-CSF mobilization or a single dose G-CSF with dexamethasone mobilization to collect products enriched in granulocytes. Cells collected in this manner are different from normal circulating granulocytes in that they contain a greater proportion of immature granulocytes that have not been well characterized regarding their lifespan. A gp91^{phox} mRNA was designed that contains a human codon-optimized open reading frame comprising pseudouridine in place of all or substantially all of the uridine residues (SEQ ID NO: 14), Xenopus laevis β-globin 5′ UTRs and 3′ UTRs and ˜100% of the mRNA molecules have a post-transcriptionally derived 5′ cap with a cap0 structure and an enzymatically extended poly(A) tail with ≥150 A's.

At 24 hours following electroporation of leukapharesis cells from an X-CGD patient with this codon-optimized gp91^{phox} mRNA (400 μg/mL), 90% of the flow scattergram-gated granulocytes expressed gp91^{phox} protein (FIG. 1C, top panels).

To demonstrate functional restoration of the mRNA-corrected granulocytes, NADPH oxidase activity was evaluated by a flow cytometric dihydrorhodamine 123 assay (DHR) to measure the granulocyte respiratory burst and the release of ROS. FACS analysis showed >80% of granulocytes were DHR positive (FIG. 1C, bottom panel), with viability of the EP leukapheresis cells greater than 80%.

### Example 4

### Transfection with p47^{phox} mRNA to Correct Autosomal-Recessive p47^{phox}-CGD Granulocytes

CGD resulting from defects in p47^{phox}, a cytosolic subunit of the NADPH oxidase, is the most common form of autosomal recessive CGD. Since p47^{phox} is a cytosolic protein and gp91^{phox} is a transmembrane protein, optimization studies for p47^{phox}, including assessments to minimize potential toxicities associated with mRNA concentrations and cell densities during electroporation, were conducted.

The p47^{phox} mRNA was prepared from a DNA template having a human codon-optimized open reading frame (SEQ ID NO: 16) and that comprises 5′ and 3′ globin UTRs, a 5′cap with a cap1 structure, pseudouridine in place of uridine and a 3′ poly(A) tail with ≥150 A's.

Stepwise optimization studies were performed on three p47^{phox} deficient patients. A comparison of the protein expression and oxidase activity revealed minimal difference in cell viabilities when monitored out to 130 hours following EP at p47^{phox} mRNA concentrations up to 400 μg/ml (FIG. 2A) or cell concentrations up to 1×10⁹/ml during EP (FIG. 2B).

Next, the kinetics of restoring p47^{phox} expression (FIG. 2C) and NADPH oxidase activity (FIG. 2D), following EP at three mRNA concentrations (200, 300, and 400 μg/ml) over time up to 120 hours, was determined. Expression of p47^{phox} was measurable in 10-35% of cells by 2 hours, reached a peak of ˜84% at 4 hours and significantly decreased by 72 hours (FIG. 2C). In contrast, improvement in NADPH oxidase activity occurred sooner after transfection with ˜90% restoration by 2 hours after transfection, and was maintained (˜75% of viable cells) out at 120 hours (FIG. 2D).

At different cell doses transfected with increasing mRNA concentrations, it was determined that mRNA concentration at 400 μg/ml achieved the highest p47^{phox} expression and NADPH oxidase activity (FIGS. 2C, 2D). Despite the low protein expression by day 6, NADPH oxidase activity was maintained at half the level even out to 5 days, indicating that it is not necessary to achieve maximum p47^{phox} protein expression to correct NADPH oxidase function. This is a remarkable outcome given that the average lifespan of human neutrophils in circulation is reported to be between 5 and 90 hours.

### Example 5

### Recruitment of Transfected Cells to Sites of Inflammation

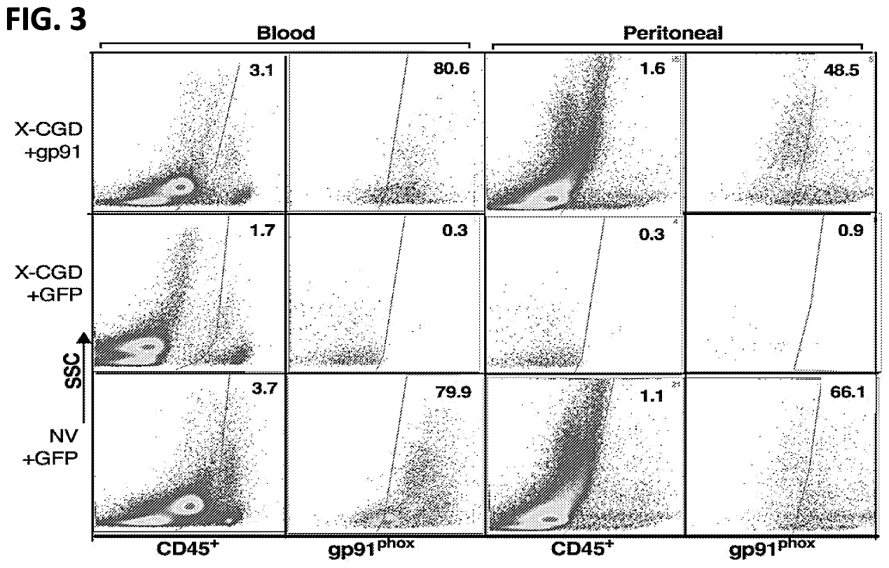

Granulocytes carry their antimicrobial cargo from the circulation into site(s) of infection or inflammation. To evaluate the in vivo chemotactic homing capability of the electroporated granulocytes following mRNA transfection, apheresed cells from X-CGD patients were EP transfected with gp91^{phox} mRNA, rested overnight, and injected intravenously via the tail vein into NonObese Diabetic gc−/−(NOD SCIDgc−) mice. Chemotactic ability and homing in vivo was determined by the detection of migration of transfected neutrophils from the bloodstream into the peritoneum following thioglycolate-induced inflammation (FIG. 3). After 6 hours, blood and peritoneal exudates were sampled from the mice that had been injected with apheresed, gp91^{phox} mRNA transfected cells from X-CGD patients and analyzed for expression of human CD45+ to identify human cells and gp9^{phox} protein as measure of transfection efficiency. As shown in FIG. 3 (top row of panels), transfected human cells were detected in blood and peritoneal exudates, demonstrating the ability of transfection corrected X-CGD cells to circulate and mobilize to inflamed site(s) in vivo. As a negative and positive controls for gp91^{phox} expression, the X-CGD cells or NV cells were EP transfected with GFP mRNA, injected intravenously in the mice, and then blood and peritoneal exudate analyzed (FIG. 3, middle row and bottom row of panels, respectively).

### Example 6

### Preclinical Dose Escalation Studies in Non-Human Primates

Pre-clinical studies in rhesus macaques were performed to assess the safety of infusing mRNA-transfected cells in non-human primates.

As discussed herein one current treatment method for CGD is a allogenic granulocyte transfusions. Granulocytes have a short half-life of 6-90 hours from egress from the marrow into the bloodstream and are prone to activation (Pillay et al., Blood 116:625-627, 2010). Recent imaging studies that tracked the migration of neutrophils to the lungs, before homing to the bone marrow, indicate a much longer life span of several days (Wang et al., Science 358:111-116, 2017). This raises concerns that transfected granulocytes may gravitate to the lungs following infusion and cause pulmonary complications. To address this concern, pre-clinical studies in non-human primates (rhesus macaques), which included serial chest imaging studies post-infusion, were performed.

Since there are no rhesus models for CGD, GFP mRNA was used for transfection into rhesus cells prepared by leukapheresis and then intravenous injection of those autologous transfected cells back into the donor monkey. To minimize immunogenic responses to foreign GFP, two animals (ZG21 and ZH32) which had previously received integrating vector GFP-transduced autologous stem cells over 4 years prior were used. These two animals had established stable, low-level percentages of GFP+ bright cells that varied between 0.2-3.0% of total cells over several years. The 8-9 kg animals were mobilized with 15 μg G-CSF/kg subcutaneously and 1 mg dexamethasone/kg intramuscularly administered approximately 12 hours prior to leukapheresis. A mobilization/leukapheresis cycle was performed on three occasions for ZG21 and on one occasion for ZH32 to collect 1×10⁷/kg, 1×10⁸/kg, 5×10⁸/kg, and 5×10⁸/kg, respectively. Following EP transfection with GFP mRNA, the apheresed cells rested overnight before reinfusion into the same animal. Following cell infusion, a chest radiograph was taken, and vital signs monitored. Peripheral blood was drawn prior to and at 5 or 30 minutes, 1 hour, 24 hours, and then daily up to 4 days (ZG21) or 5 days (ZH32) following infusions to evaluate GFP expression in circulating blood cells.

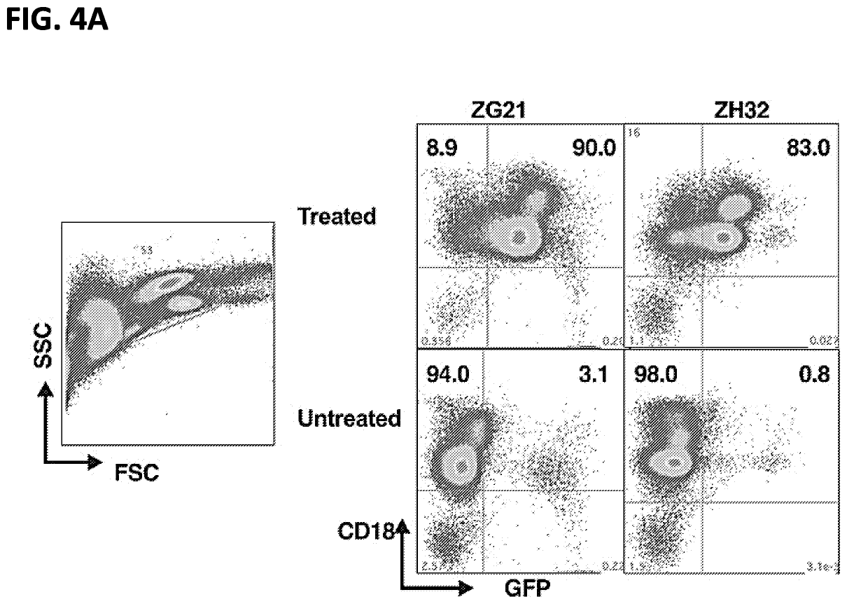

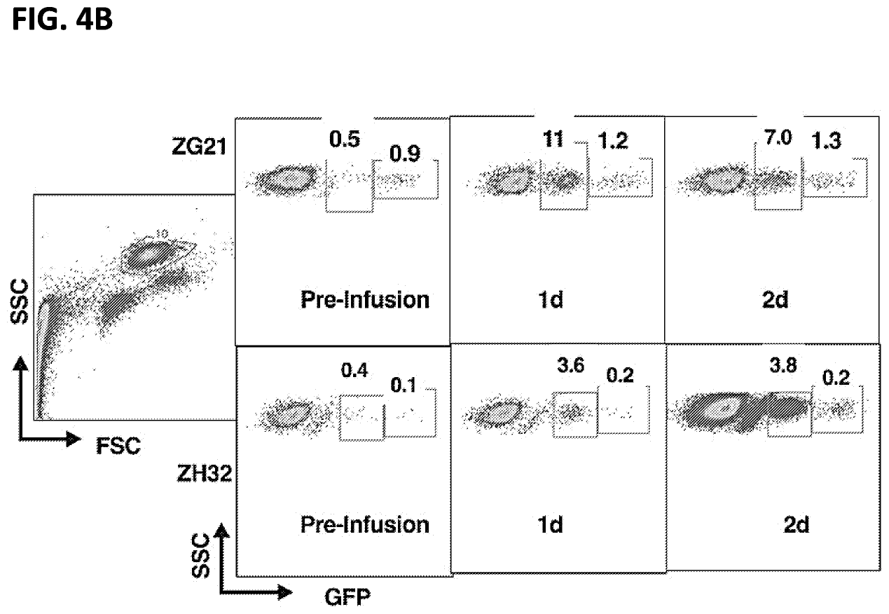

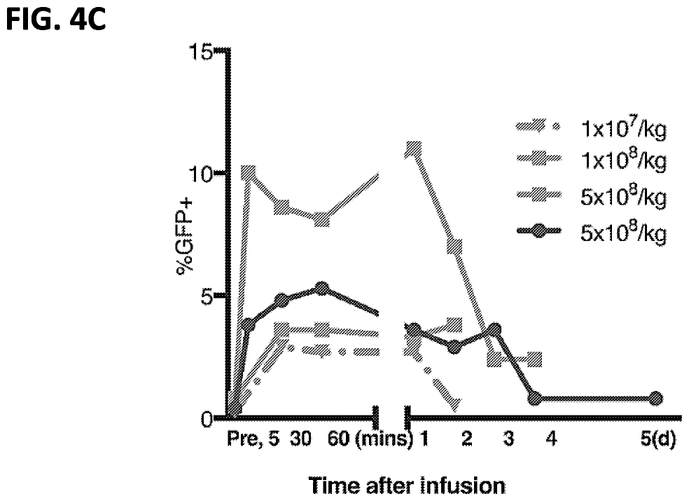

The animals had a small percent of baseline bright level GFP expression that was clearly distinguishable from the less bright level GFP expression due to exogenous GFP mRNA transfection, where the baseline GFP expression location in the dot plots is most evident in the ex vivo “Untreated” apheresis samples (FIG. 4A) and in the “Pre-infusion” blood samples from the in vivo studies (FIG. 4B). There were no adverse reactions nor changes in vital signs or general condition of the animals as observed for 4-5 days post infusion. Autologous leukapheresis products collected from rhesus macaques that were EP transfected with GFP mRNA showed GFP expression (˜83-90% of cells) in the CD18+ granulocyte/monocyte “Treated” population (FIG. 4A).

GFP expression in circulating blood granulocytes following infusion of autologous transfected leukapheresis products was monitored by flow cytometry for GFP expression as shown in FIG. 4B pre-infusion and at 1 and 2 days after infusions in each of the two animals as indicted (FIG. 4B). In the FIG. 4B panels measuring GFP, the less bright GFP expression due to the GFP mRNA transfection (left most boxed area) was clearly distinguishable from the baseline bright GFP expression (right-most boxed area) in granulocytes (FIG. 4B).

A more detailed assessment of GFP expression over time after each of 4 infusions (at 5 minutes, 30 minutes, 1 hour, and then daily up to 5 days) is shown in FIG. 4C. The data showed early detection of significant numbers of (3.8%-10%) circulating transfection-related GFP+ granulocytes at 5 minutes after the infusion, maintained or peaked at 24 hours, decreasing slightly to modestly at day 2-3, and was still detectable at low levels on day 4 or 5 (FIG. 4C). One animal (ZG21) received three collections and infusions of GFP mRNA transfected cells on separate occasions and tolerated all three collections and infusions without any adverse events.

### Example 7

### Cell Manufacturing Controls

In support of a clinical protocol to treat patients with mRNA-corrected autologous peripheral blood granulocytes, a validation run was performed under clinical conditions using pharmaceutical grade (GMP-manufacturing conditions) gp91^{phox} mRNA.

Cells were collected by apheresis from an adult patient with X-CGD following 5 days of G-CSF and one dose of plerixafor to yield a product with 40% granulocytes that was further concentrated to 69% by elutriation. This product was not a standard granulocyte collection but was a sub-portion of a research hematopoietic stem cell mobilization/apheresis collection from an adult patient enrolled in an clinical trial so as not to subject this subject to a separate granulocyte-only collection. 3×10⁹ cells (equivalent to a 1×10⁸ cells/kg dose for a hypothetical 30 kg pediatric patient) were transfected with 400 g gp91^{phox} mRNA/mL.

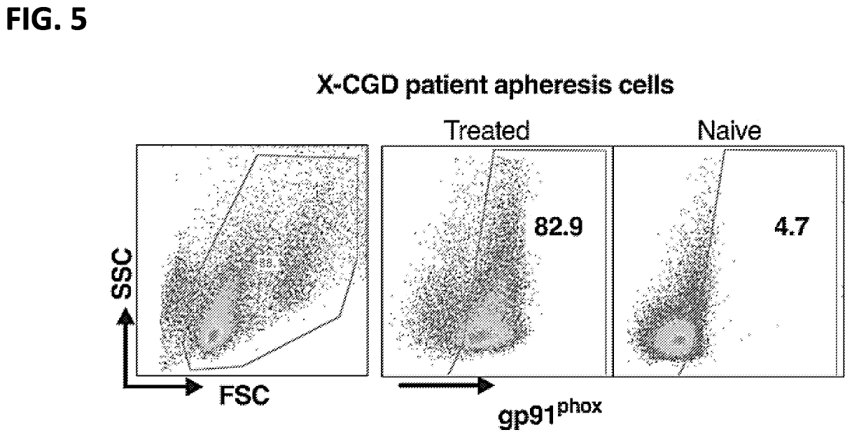

As shown in FIG. 5, this clinical scale-up resulted in 88% cell viability and 83% gp91^{phox} expressing cells at 18 hours post-transfection.

### Example 8

### MAGT1 mRNA Transfection of XMEN Patient Lymphocytes and NK Cells

The disclosed methods for correcting primary blood cells with mRNA transfection were used to treat XMEN disease, which affects primarily lymphocytes and NK cells.

To treat a subject with XMEN, expression of magnesium transporter 1 (MAGT1) is needed to allow proper activation and function of lymphocytes. An exemplary measure of the successful restoration of MagT1 function is the expression of NKG2D on the surface of CD8+ T cells (such as an increase of at least 20%, at least 30%, at least 40%, at least 50%, at least 75%, at least 80%, at least 90%, at least 100%, at least 2-fold, at least 5-fold, or at least a 10-fold increase as compared to an amount of NKG2D expression on CD8+ T cells prior to administration of lymphocytes expressing MagT1). Expression of NKG2D can be measured by a flow cytometry using NKG2D-specific antibodies. With normal physiologic control, NKG2D should appear only on CD8⁺ T cells and not on CD4⁺ T cells.

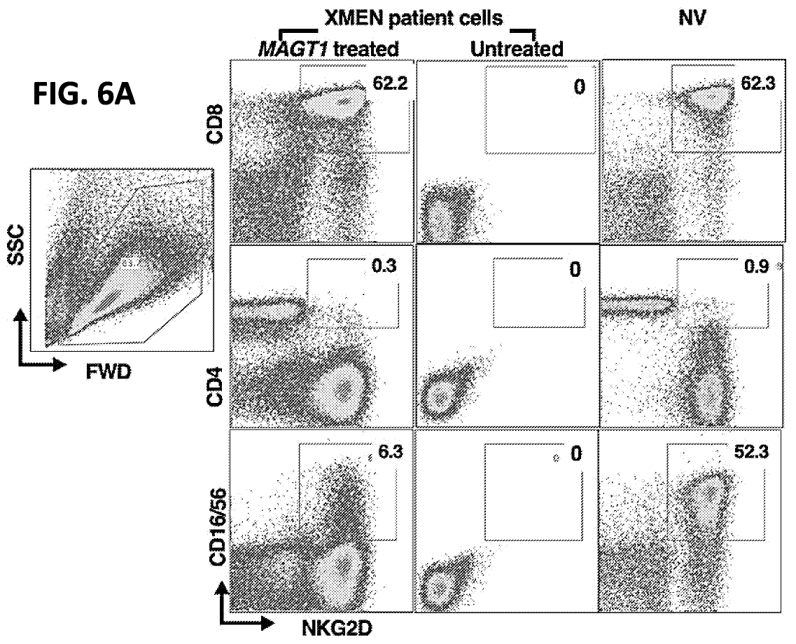

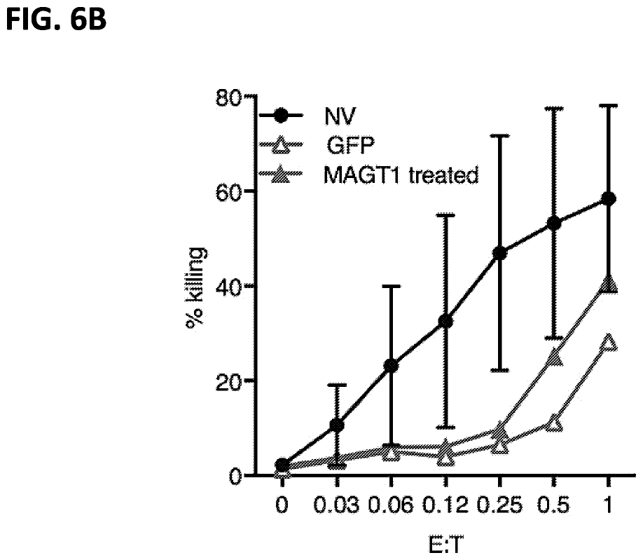

Leukapheresis was performed on patients with XMEN disease adjusting the apheresis collection to enrich for mononuclear cells. Unlike granulocytes, lymphocytes and NK cells in the apheresis product survive cryogenic storage for long term use. Thawed leukapheresis products containing both lymphocytes and NK cells were transfected with MAGT1 mRNA using electroporation conditions optimized for lymphocytes (MaxCyte Inc.) and applied either prior to proliferation, or following expansion of lymphocytes (using CD3/CD28 beads) (FIG. 6A) or NK cells. Since there are no commercially available monoclonal antibodies for flow cytometric detection of MAGT1 protein, and expression of NKG2D is dependent on presence of functional MAGT1, NKG2D expression was used as a functional biomarker of MAGT1 correction. Following transfection with a MAGT1 mRNA (SEQ ID NO: 18) comprising 5′ and 3′ globin UTRs, a 5′cap with a cap1 structure, pseudouridine in place of uridine and a 3′ poly(A) tail with ≥150 A's, NKG2D expression was restored specifically on CD8⁺ T cells and NK cells, while similarly treated CD4⁺ T cells did not express NKG2D. This specific NKG2D expression pattern ensures that the exogenous MAGT1 mRNA does not, in this case, alter the protein expression of NKG2D in relevant cells as observed in normal volunteer cells (FIG. 6A).

To evaluate the corrected NK cell function, transfected XMEN patient NK cells were co-cultured with NK-susceptible K562 erythroleukemia cells at several effector:target ratios. Although patient cells did not fully normalize killing, there was an improved cytotoxicity by MAGT1 mRNA transfected NK cells was observed (FIG. 6B).

This data demonstrates that transfection of MAGT1 mRNA corrected downstream function by restoring the expression of NKG2D that is needed for antiviral immunity and clearance of transformed cells. Since there is currently no effective treatment for XMEN disease, this method provides a new and needed therapeutic approach in this disease. For example, such transfected lymphocyte cells can be administered to a subject with XMEN (e.g., autologous granulocytes, NK cells, and/or lymphocytes can be transfected with a MAGT1 mRNA, and introduced into the subject, for example to treat XMEN and/or to treat an infection or autoimmune disease in the XMEN subject).

### Example 9

### Treatment of Humans Using Recombinant Autologous Granulocytes Expressing gp91phox

Human subjects with X-CGD undergo apheresis following 5 days of G-CSF stimulation without the aid of HES. The resulting apheresis product is transfected with clinical grade gp91phox mRNA (SEQ ID NO: 14, having a human codon-optimized open reading frame and that further comprises 5′ and 3′ globin UTRs, a 5′cap with a cap0 structure, and a 3′ poly(A) tail with ≥150 A's) in vitro transcribed from a plasmid encoding codon-optimized CYBB cDNA (CELLSCRIPT, LLC) on the same day, kept in culture overnight, and transfused back into the subject as a single intravenous infusion the following day (Day 0) as shown in the schema below.

Following infusion, the subject will be monitored closely for 5 days for adverse reactions and peripheral blood circulating oxidase normal neutrophils monitored. At three months following transfusion of the recombinant autologous granulocytes, a follow up visit to examine the subject and safety labs will be performed.

In Phase 1, autologous granulocytes will be collected by apheresis without the use of hydroxyethyl starch following daily G-CSF 5-15 mcg/kg body mass for 5 days. The target dose in Phase 1 is 5×10⁶ recombinant autologous granulocytes/kg (in a volume of 50-100 mL intravenously over 30-45 minutes). Test doses of 5×10⁶ recombinant autologous granulocytes/kg body weight will be administered in Phase I to assess tolerability of transfected granulocyte transfusions. The recombinant autologous granulocytes are infused as a single infusion, with a small aliquot put aside for in vitro analysis. If no recombinant autologous granulocytes (5×10⁶ recombinant autologous granulocytes/kg) related adverse events occur in all 3 subjects, the study can proceed to the higher dose of 5×10⁷ recombinant autologous granulocytes/kg in Phase 2. If a subject in Phase 1 develops a recombinant autologous granulocyte related adverse event, an additional 3 subjects will be treated with the test dose. Progression to Phase 2 may only take if there are no more than two recombinant autologous granulocytes related adverse events. The test doses in Phase 1 are not administered more frequently than once a week.

In Phase 2, the target dose is 5×10⁶ recombinant autologous granulocytes/kg (in a volume of 50-100 mL intravenously over 30-45 minutes). A moderate dose of 5×10⁷ recombinant autologous granulocytes/kg body weight will be transfused to assess tolerability and reconstitution in 3 subjects. The recombinant autologous granulocytes are infused as a single infusion, with a small aliquot put aside for in vitro analysis. In Phase 2, recombinant autologous granulocytes are not administered more than once per week. If a subject in Phase 2 develops a recombinant autologous granulocytes-related adverse event, an additional 3 subjects will be treated with the test dose. Progression to Phase 3 may only take if there are no more than two recombinant autologous granulocytes-related adverse events.

In Phase 3, subjects will be administered maximum treatment doses (e.g., the full apheresis product (usually between 0.5-4×10¹⁰ cells in total or up to ˜5×10⁸ recombinant autologous granulocytes/kg for a 50 kg adult) following transfection. The recombinant autologous granulocytes are infused as a single infusion (in a volume of 50-100 mL intravenously over 30-45 minutes), with a small aliquot put aside for in vitro analysis. In Phase 3, the recombinant autologous granulocytes can be delivered up to 2 times a week, for up to a total of 6 doses for any subject. If a recombinant autologous granulocytes-related adverse event occurs in 2 out of 6 subjects, or in 3 out of 9 subjects, the protocol will pause. Phase 3 subjects who receive ≥6 treatment doses will also be evaluated for clinical benefits of the recombinant autologous granulocyte infusions.

The recombinant autologous granulocytes transfected with mRNA has a very short half-life of 6-8 hours. Therefore, it is expected that adverse events related to the infusion of the recombinant autologous granulocytes will occur by three days following the infusion.

40 full treatment doses (1-5×10¹⁰ TNC; ˜5×10⁸/kg for a 50 kg adult) of the recombinant autologous granulocytes can be administered, which can be repeated in any subject with an intent to treat active infections as an adjunct to ongoing treatment.

Patients are monitored one week from the time of G-CSF stimulation to completion of post-transfusion monitoring, and a follow-up visit 3-6 months later for safety labs.

A summary of the progression is provided in Table 2 below:

### Table 2: Clinical Trial Progression

TABLE 2
Clinical trial progression

| Outcome: # of Dose Related | |
|---|---|
| Toxicities (DRT) out of # of | |
| Subjects at a Given Dose level | Decision Rule |
| 0 DRT out of 3 subjects | Enter up to 3 subjects at next |
| | dose level |
| 2 DRT out of 2-3 subjects | Stop dose escalation: Enter up to |
| | 3 additional subjects the previous |
| | dose level if only 3 subjects have |
| | been treated at that dose. |
| 1 DRT out of 3 subjects | Enter up to 3 more subjects at the |
| | same dose level |
| 1 DRT out of 6 subjects | Enter up to 3 subjects at the next |
| | dose level. |
| 2 DRTs out of 4-6 subjects | Stop dose escalation: Enter up to |
| | 3 additional subjects at the |
| | previous dose level if only 3 |
| | subjects have been treated at that |
| | dose. |

To determine the kinetics of recombinant autologous granulocyte infusion, serial measurements of NADPH oxidase function by DHR and/or gp91phox expression (e.g., by staining peripheral blood cells using flow cytometry) in peripheral blood granulocytes will be determined following the infusion, for example at pre-infusion, 30 minutes, 1 hour, 2 hours, 4 hours, 12 hours, 16 hours, 24 hours (1 day), 2 days, 3 days, 4 days and 5 days post infusion.

Infection markers (e.g., labs such as white blood count, inflammatory markers, imaging such as ultrasounds, CT scans, MRI, physical visualization of skin lesions) to assess progress of infection-weight, fevers, Erythrocyte sedimentation rate, C-reactive protein, microbiological and clinical imaging studies. For subjects who receive ≥6 treatment doses in Phase 3, a formal assessment for infection status will be performed.

The following blood laboratory analyses can be performed on the subjects before, during, and/or after the transfusion of recombinant autologous granulocytes.
Hematology: Complete blood count (CBC) including Red Blood Cells (RBC), hemoglobin, hematocrit, platelets, White Blood Cells (WBC) and differential.
Chemistry panel: Na, K, Cl, CO₂, Creat, BUN, AST, ALT, T. bili, Ca, total protein, albumin
Other tests: CGD panel which includes dihydrorhodamine assay (DHR) to assess the NADPH oxidase activity of the peripheral blood neutrophils, and the level of gp91phox protein expression by flow cytometric analysis. Store serum and mononuclear cells for subsequent analysis, for example, cytokines

Before leukapheresis, and following infusion of the recombinant autologous granulocytes, the subject can be monitored for:
vital signs, temperature, BP every 15 minutes for the first hour, every hour for 4 hours, then every 6-8 hours until 3-5 days post transfusion.
CBC/chem/liver and mineral panels daily for 3 days
Blood collected for CGD panel (DHR, or gp91phox expression, or chemiluminescence) at pre-, post at 30′, 60″, 2 h, 4 h. 8 h, 24 h+/−1 hour, daily until day 5 after transfusion. Monitoring of CGD panel ceases if below level of sensitivity of detection (≤1%) DHR+ granulocytes are detected
Research specimens (PBMC and serum) collected daily before and for 3 days after recombinant autologous granulocytes infusion for cryopreservation and future assays, for example, cytokine profile.

An overview of the procedures is provided in Table 3.

The patients will return for a follow-up visit at 3 to 6 months, for a full history and physical examination, interim history, complete blood count, chemistry, ‘CGD panel’, and evaluation of infection status.

In some examples, the recombinant autologous granulocytes have DHR+ or gp91phox expression (as detected by FACS analysis) in at least 20% of granulocytes in vitro. In some examples, treated subjects have, in their peripheral blood, detectable (e.g., at least 2%, at least 3%, at least 4%, or at least 5%) oxidase positive circulating granulocytes following donor granulocyte transfusion, after a 5×10⁸/kg cell dose.

| | | | Day | Day | Day | Day | 3 | 6 |
|---|---|---|---|---|---|---|---|---|
| | | | 0 | 1 | 2 | 3 | Months | Months |
| | | | Cell | post cell | post cell | post cell | Follow | Follow |
| Evaluation | Screening | Baseline | Infusion | infusion | infusion | infusion | up visit | up visit |
| Medical/Medication | X | X | | | | | X | X |
| History | | | | | | | | |
| Clinical Evaluation: | X | X | | | | | X | X |
| History and Physical, | | | | | | | | |
| Weight, and Safety | | | | | | | | |
| assessment | | | | | | | | |
| CBC with differential | X | X | | X | X | X | X | X |
| Serum chemistries | X | X | | X | X | X | | |
| (Na, K, Cl, C02, | | | | | | | | |
| Creat, BUN, AST, | | | | | | | | |
| ALT, T.bili, Ca, Total | | | | | | | | |
| protein, albumin) | | | | | | | | |
| PT/PTT | x | x | | | | | | |
| Vital signs | X | X | | X | X | X | X | X |
| Research | | | | | | | | |
| gp91phox- | | X | | X | X | X | | |
| (expression by FACS) | | | | | | | | |
| PBMC and Serum for | | X | | X | X | X | | |
| Storage | | | | | | | | |
| CGD Panel (DHR, gp | | X | | X | X | X | | |
| 91 phox or | | | | | | | | |
| Chemiluminescence) | | | | | | | | |

### Example 10

### Expression of CTLA-4 in PBMCs

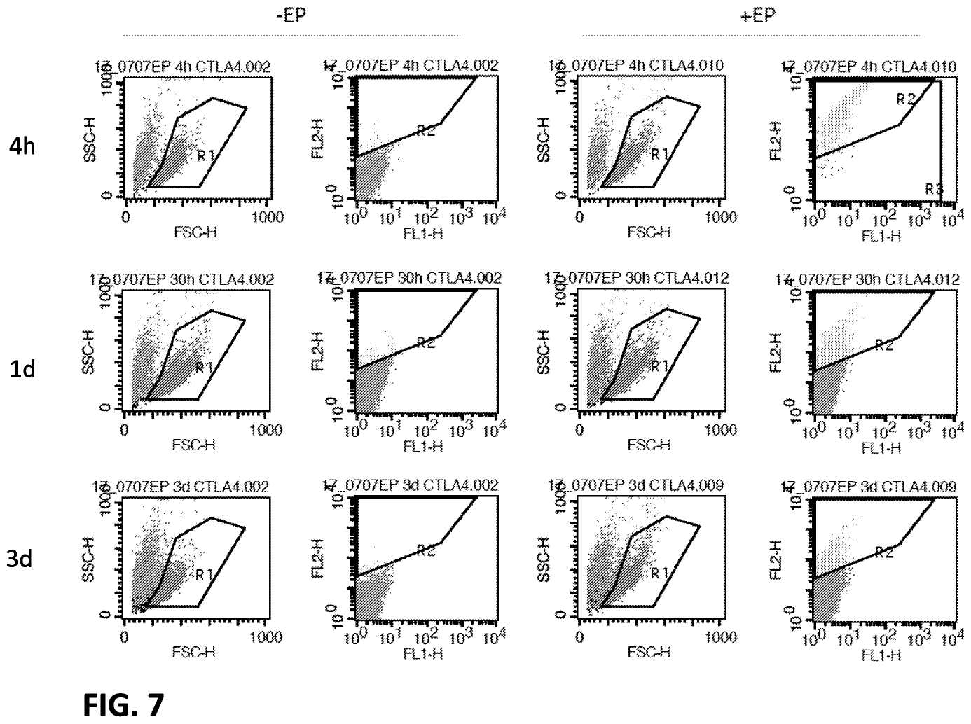

PBMCs were isolated from a patient with CLTA-4 deficiency and cultured for 7-10 days in RPMI, fetal calf serum, and IL-2. An mRNA encoding CLTA-4, which included a 5′-end cap and 3′-poly A tail) was transfected into the PBMCs using electroporation. Expression of CLTA-4 was monitored using flow cytometry at 4 hours, 1 day, and 3 days post-transfection. As shown in FIG. 7, expression of CTLA-4 in the PBMCs was achieved.

Such cells can be administered to a subject with CTLA-4 deficiency (e.g., autologous granulocytes, NK cells, and/or lymphocytes can be transfected with a CTLA-4 encoding mRNA, and introduced into the subject, for example to treat an infection or autoimmune disease in the subject).

In view of the many possible embodiments to which the principles of the disclosure may be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

1. A method of treating a primary immunodeficiency disease (PID) in a subject, comprising:
administering a therapeutically effective first dose of recombinant autologous granulocytes, recombinant autologous NK cells, and/or recombinant autologous lymphocytes into the subject, wherein the recombinant autologous granulocytes, recombinant autologous NK cells, and/or recombinant autologous lymphocytes comprise one or more exogenous messenger ribonucleic acids (mRNAs) that encode at least one protein deficient in the subject due to the PID; and
expressing the at least one protein from the one or more exogenous mRNAs in the recombinant autologous granulocytes, recombinant autologous NK cells, and/or recombinant autologous lymphocytes, thereby treating the PID in the subject.

2. A method of treating a chronic infection, autoimmune disease, immune dysregulation, or combinations thereof, in a subject having a primary immunodeficiency disease (PID), comprising:
administering a therapeutically effective first dose of recombinant autologous granulocytes, recombinant autologous NK cells, and/or recombinant autologous lymphocytes into the subject, wherein the recombinant autologous granulocytes, recombinant autologous NK cells, and/or recombinant autologous lymphocytes comprise one or more exogenous mRNAs that encode at least one protein deficient in the subject due to the PID; and
expressing the at least one protein from the one or more exogenous mRNAs in the recombinant autologous granulocytes, recombinant autologous NK cells, and/or recombinant autologous lymphocytes, thereby treating the infection, immune dysregulation, autoimmune disease, or combinations thereof, in the subject.

3. The method of claim 2, wherein
the chronic infection is a bacterial, fungal, parasitic, or viral infection,
the autoimmune disease is rheumatoid arthritis, Hashimoto's thyroiditis, pernicious anemia, inflammatory bowel disease, psoriasis, renal, pulmonary, and hepatic fibroses, Addison's disease, type I diabetes, systemic lupus erythematosus, dermatomyositis, Sjogren's syndrome, multiple sclerosis, myasthenia gravis, Reiter's syndrome, or Grave's disease, or
combinations thereof.

4. The method of claim 1, further comprising administering a second dose of the recombinant autologous granulocytes, recombinant autologous NK cells, and/or recombinant autologous lymphocytes into the subject.

5. The method of claim 1, further comprising:
transfecting autologous granulocytes, autologous NK cells, and/or autologous lymphocytes with the one or more exogenous mRNAs, thereby generating the recombinant autologous granulocytes, recombinant autologous NK cells, and/or recombinant autologous lymphocytes.

6. The method of claim 1, wherein the subject undergoes apheresis to obtain the autologous granulocytes, autologous NK cells, and/or autologous lymphocytes.

7. (canceled)

8. The method of claim 6, wherein the apheresis is performed without hydroxyethyl starch (HES).

9. The method of claim 1, further comprising administering to the subject
a hematopoietic stem cell (HSC) transplant or bone marrow transplant; and/or
an antiviral agent, an antifungal agent, and/or an antibiotic agent.

10. (canceled)

11. The method of claim 1, wherein the PID is a
monogenic PID;
phagocytic disorder;
chronic granulomatous disease (CGD), the protein deficient is NADPH oxidase, and the one or more exogenous mRNAs encode one or more of gp91phox, p47phox, p67phox, p22phox, and p40phox; or
lymphocytic disorder.

12.-13. (canceled)

14. The method of claim 1, wherein the subject has CGD and is infected with Staphylococcus aureus, Serratia marcescens, Burkholderia cepacia complex, Listeria, E. coli, Klebsiella, Pseudomonas cepacia, Nocardia, Aspergillus, or combinations thereof.

15. (canceled)

16. The method of claim 1, wherein the PID is X-linked magnesium defect, Epstein-Barr virus infection and neoplasia (XMEN) and the protein deficient is magnesium transporter 1 (MagT1), and the mRNA encodes MagT1.

17. (canceled)

18. The method of claim 1, wherein the autologous granulocytes, autologous NK cells, and/or autologous lymphocytes are obtained from a blood sample of the subject.

19. (canceled)

20. The method of claim 1, wherein the one or more exogenous mRNAs
comprise a 5′-end cap;
comprise a 3′-end poly-A tail;
comprise a 5′-end cap and a 3′-end poly-A tail;
comprise a 3′-untranslated region (UTR);
comprise a 5′-UTR;
comprises one or more pseudouridines in place of one or more uridines;
comprises one or more non-naturally occurring nucleosides in place of one or more uridines;
is codon optimized for expression in a human cell;
comprises a 5′-end cap, comprises a 3′-end poly-A tail, comprises one or more pseudouridines in place of one or more uridines, and is codon optimized for expression in a human cell;
comprises a 5′-end cap, comprises a 5′-UTR, comprises a 3′-UTR, comprises a 3′-end poly-A tail, comprises one or more pseudouridines in place of one or more uridines, and is codon optimized for expression in a human cell;
or combinations thereof.

21. The method of claim 1, wherein the one or more exogenous mRNAs encode:
a gp91 protein, wherein the gp91 protein comprises at least 80%, at least 85%, at least 90%, at least 95%, at least 98%, or at least 99% sequence identity to SEQ ID NO: 5 or 15;
a p47phox protein, wherein the p47phox protein comprises at least 80%, at least 85%, at least 90%, at least 95%, at least 98%, or at least 99% sequence identity to SEQ ID NO: 9 or 17;
a p67phox protein, wherein the p67phox protein comprises at least 80%, at least 85%, at least 90%, at least 95%, at least 98%, or at least 99% sequence identity to SEQ ID NO: 11 or 23;
a p22phox protein, wherein the p22phox protein comprises at least 80%, at least 85%, at least 90%, at least 95%, at least 98%, or at least 99% sequence identity to SEQ ID NO: 7 or 21;
a p40phox protein, wherein the p40phox protein comprises at least 80%, at least 85%, at least 90%, at least 95%, at least 98%, or at least 99% sequence identity to SEQ ID NO: 13 or 25;
a CTLA4 protein, wherein the CTLA4 protein comprises at least 80%, at least 85%, at least 90%, at least 95%, at least 98%, or at least 99% sequence identity to SEQ ID NO: 27;
a Magt1 protein, wherein the Magt1 protein comprises at least 80%, at least 85%, at least 90%, at least 95%, at least 98%, or at least 99% sequence identity to SEQ ID NO: 3 or 19;
or combinations thereof.

22. The method of claim 1, wherein
the one or more exogenous mRNAs encode gp91, and a coding portion of the mRNA comprises at least 60%, at least 70%, at least 75%, at least 80%, at least 85%, at least 90%, at least 95%, at least 98%, or at least 99% sequence identity to SEQ ID NO: 1, 4, or 14;
the one or more exogenous mRNAs encode p47phoxA, and a coding portion of the mRNA comprises at least 60%, at least 70%, at least 75%, at least 80%, at least 85%, at least 90%, at least 95%, at least 98%, or at least 99% sequence identity to SEQ ID NO: 8 or 16;
the one or more exogenous mRNAs encode p67phox, and a coding portion of the mRNA comprises at least 60%, at least 70%, at least 75%, at least 80%, at least 85%, at least 90%, at least 95%, at least 98%, or at least 99% sequence identity to SEQ ID NO: 10 or 22;
the one or more exogenous mRNAs encode p22phox, and a coding portion of the mRNA comprises at least 60%, at least 70%, at least 75%, at least 80%, at least 85%, at least 90%, at least 95%, at least 98%, or at least 99% sequence identity to SEQ ID NO: 6 or 20;
the one or more exogenous mRNAs encode p40phox, and a coding portion of the mRNA comprises at least 60%, at least 70%, at least 75%, at least 80%, at least 85%, at least 90%, at least 95%, at least 98%, or at least 99% sequence identity to SEQ ID NO: 12 or 24;
the one or more exogenous mRNAs encode CTLA4, and a coding portion of the mRNA comprises at least 60%, at least 70%, at least 75%, at least 80%, at least 85%, at least 90%, at least 95%, at least 98%, or at least 99% sequence identity to SEQ ID NO: 26;
the one or more exogenous mRNAs encode Magt1, and a coding portion of the mRNA comprises at least 60%, at least 70%, at least 75%, at least 80%, at least 85%, at least 90%, at least 95%, at least 98%, or at least 99% sequence identity to SEQ ID NO: 2 or 18;
or combinations thereof.

23. A recombinant autologous granulocyte, recombinant autologous NK cell, or recombinant autologous lymphocyte expressing
an mRNA encoding a gp91 protein, wherein the gp91 protein comprises at least 80%, at least 85%, at least 90%, at least 95%, at least 98%, or at least 99% sequence identity to SEQ ID NO: 5 or 15;
an mRNA encoding a p47phox protein, wherein the p47phox protein comprises at least 80%, at least 85%, at least 90%, at least 95%, at least 98%, or at least 99% sequence identity to SEQ ID NO: 9 or 17;
an mRNA encoding a p67phox protein, wherein the p67phox protein comprises at least 80%, at least 85%, at least 90%, at least 95%, at least 98%, or at least 99% sequence identity to SEQ ID NO: 11 or 23;
an mRNA encoding a p22phox protein, wherein the p22phox protein comprises at least 80%, at least 85%, at least 90%, at least 95%, at least 98%, or at least 99% sequence identity to SEQ ID NO: 7 or 21;
an mRNA encoding a p40phox protein, wherein the p40phox protein comprises at least 80%, at least 85%, at least 90%, at least 95%, at least 98%, or at least 99% sequence identity to SEQ ID NO: 13 or 25;
an mRNA encoding a CTLA4 protein, wherein the CTLA4 protein comprises at least 80%, at least 85%, at least 90%, at least 95%, at least 98%, or at least 99% sequence identity to SEQ ID NO: 27;
an mRNA encoding a Magt1 protein, wherein the Magt1 protein comprises at least 80%, at least 85%, at least 90%, at least 95%, at least 98%, or at least 99% sequence identity to SEQ ID NO: 3 or 19;
or combinations thereof,
wherein the granulocyte, NK cell, or lymphocyte prior to becoming recombinant is from a subject with a PID.

24. The recombinant autologous granulocyte, recombinant autologous NK cell, or recombinant autologous lymphocyte of claim 23, wherein
the mRNA encodes gp91, and a coding portion of the mRNA comprises at least 60%, at least 70%, at least 75%, at least 80%, at least 85%, at least 90%, at least 95%, at least 98%, or at least 99% sequence identity to SEQ ID NO: 1, 4, or 14;
the mRNA encodes p47phoxA, and a coding portion of the mRNA comprises at least 60%, at least 70%, at least 75%, at least 80%, at least 85%, at least 90%, at least 95%, at least 98%, or at least 99% sequence identity to SEQ ID NO: 8 or 16;
the mRNA encodes p67phox, and a coding portion of the mRNA comprises at least 60%, at least 70%, at least 75%, at least 80%, at least 85%, at least 90%, at least 95%, at least 98%, or at least 99% sequence identity to SEQ ID NO: 10 or 22;
the mRNA encodes p22phox, and a coding portion of the mRNA comprises at least 60%, at least 70%, at least 75%, at least 80%, at least 85%, at least 90%, at least 95%, at least 98%, or at least 99% sequence identity to SEQ ID NO: 6 or 20;
the mRNA encodes p40phox, and a coding portion of the mRNA comprises at least 60%, at least 70%, at least 75%, at least 80%, at least 85%, at least 90%, at least 95%, at least 98%, or at least 99% sequence identity to SEQ ID NO: 12 or 24;
the mRNA encodes CTLA4, and a coding portion of the mRNA comprises at least 60%, at least 70%, at least 75%, at least 80%, at least 85%, at least 90%, at least 95%, at least 98%, or at least 99% sequence identity to SEQ ID NO: 26;
the mRNA encodes Magt1, and a coding portion of the mRNA comprises at least 60%, at least 70%, at least 75%, at least 80%, at least 85%, at least 90%, at least 95%, at least 98%, or at least 99% sequence identity to SEQ ID NO: 2 or 18;
or combinations thereof.

25. A recombinant autologous granulocyte, recombinant autologous NK cell, or recombinant autologous lymphocyte expressing
an mRNA encoding a protein listed in Table 1,
wherein the granulocyte, NK cell, or lymphocyte prior to becoming recombinant is from a subject with a corresponding PID listed in Table 1.

26. The recombinant autologous granulocyte, recombinant autologous NK cell, or recombinant autologous lymphocyte of claim 23, wherein the mRNA
comprises a 5′-end cap;
comprises a 3′-end poly-A tail;
comprises a 5′-end cap and a 3′-end poly-A tail;
comprises a 3′-UTR;
comprises a 5′-UTR;
comprises one or more pseudouridines in place of one or more uridines;
comprises one or more non-naturally occurring nucleotides in place of one or more uridines;
is codon optimized for expression in a human cell;
comprises a 5′-end cap, comprises a 3′-end poly-A tail, comprises one or more pseudouridines in place of one or more uridines, and is codon optimized for expression in a human cell;
comprises a 5′-end cap, comprises a 5′-UTR, comprises a 3′-UTR, comprises a 3′-end poly-A tail, comprises one or more pseudouridines in place of one or more uridines, and is codon optimized for expression in a human cell;
or combinations thereof.

27. A composition comprising:
the recombinant autologous granulocyte, recombinant autologous NK cell, or recombinant autologous lymphocyte of claim 23; and
a pharmaceutically acceptable carrier, a cell culture medium, or DMSO.

28.-32. (canceled)
